(19) United States
(12) Patent Application Publication
(10) Pub. No.: US 2026/0027105 A1
(43) Pub. Date: Jan. 29, 2026

(54) COMPOUND MEDICINE COMPOSITION FOR TREATING GLAUCOMA AND USE OF SAME

(71) Applicant: Shenyang Pharmaceutical University, Shenyang (CN)

(72) Inventors: Qikun JIANG, Shenyang (CN); Tao WANG, Shenyang (CN); Meiling CHI, Shenyang (CN); Chen ZHAO, Shenyang (CN)

(73) Assignee: Shenyang Pharmaceutical University, Shenyang (CN)

(21) Appl. No.: 18/948,680

(22) Filed: Nov. 15, 2024

Related U.S. Application Data
(63) Continuation of application No. PCT/CN2023/093497, filed on May 11, 2023.

(30) Foreign Application Priority Data
May 18, 2022 (CN) 202210550653.1

(51) Int. Cl. A61K 31/472; A61K 9/00; A61K 9/08; A61K 31/138; A61K 31/24; A61K 31/4704; A61K 31/5377; A61K 31/5575; A61K 47/02; A61K 47/18; A61K 47/26; A61P 27/06

(52) U.S. Cl. CPC A61K 31/472; A61K 9/0048; A61K 9/08; A61K 31/138; A61K 31/24; A61K 31/4704; A61K 31/5377; A61K 31/5575; A61K 47/02; A61K 47/186; A61K 47/26; A61P 27/06

(57) ABSTRACT

The present disclosure provides ophthalmic dual and triple compound medicine compositions for treating glaucoma and ocular hypertension. Through a salt modification technology, stability of the medicine compositions is improved remarkably. The dual compound medicine composition is prepared from netarsudil free alkali or pharmaceutically acceptable salts of the same and a β-adrenergic receptor blocker. The triple compound medicine composition is prepared from the netarsudil free alkali or the pharmaceutically acceptable salts of the same, the β-adrenergic receptor blocker and a prostaglandin analog. The dual and triple compound medicine compositions have excellent stability when pH is in a range from 4.5 to 5.4, an effective dose of the medicine composition of the present disclosure is applied to eyes of a patient in need once a day at bedtime or close to bedtime, an intra-ocular pressure can be reduced efficiently and quickly, the intra-ocular pressure remains in a physiological range for a longer time, side effects are minor, and patient compliance is high.

### CROSS-REFERENCE TO RELATED APPLICATIONS

This application is a continuation of PCT application number PCT/CN2023/093497, filed on May 11, 2023, which claims priority to Chinese application number 202210550653.1, filed on May 18, 2022, the disclosures of which are incorporated herein by reference in their entirety.

### FIELD

The present disclosure belongs to the field of medicine and particularly relates to ophthalmic dual and triple compound medicine compositions and use of the same in preparation of medicine for preventing or treating glaucoma or medicine for reducing an intra-ocular pressure.

### BACKGROUND OF THE INVENTION

Glaucoma is a common ophthalmic disease, which is brought on due to primary risk factors of pathological increasing of intra-ocular pressure and insufficient blood supply to an optic nerve. Increasing of intra-ocular pressure causes pressure on the optic nerve, which makes an optic nerve fiber layer thinner, damages optic nerve cells and then leads to diminution of vision, a visual field defect, optic neuratrophy, etc., and a patient with severe glaucoma may even suffer from eye blindness, so glaucoma has become a leading killer for human blindness.

At present, there are several types of common unilateral preparations as follows: (1) an Rho kinase inhibitor: it is medicine for reducing an intra-ocular pressure which directly acts on a trabecular meshwork and changes cell morphology, cell movement, cytoplasmic division, smooth muscle contraction, etc. of the trabecular meshwork mainly by affecting cytoskeleton, so as to increase aqueous humor outflow and reduce the intra-ocular pressure; and meanwhile, the Rho kinase inhibitor plays a role in improving retina vascular perfusion, facilitating optic nerve regeneration, protecting the optic nerve, reducing filtering bleb scarring, etc. A drug commonly used in clinic is netarsudil mesylate. (2) A β-adrenergic receptor blocker: it may reduce generation of aqueous humor and facilitate aqueous humor drainage and discharge by blocking the β receptor, has a powerful and lasting effect on reducing the intra-ocular pressure, and effectively controls the intra-ocular pressure. Drugs commonly used in clinic are timolol maleate, carteolol hydrochloride, betaxolol hydrochloride, metipranolol hydrochloride, etc. (3) A prostaglandin analog: it reduces the intra-ocular pressure by relaxing ciliary muscle, widening intermuscular space and increasing outflow and drainage of a uveoscleral pathway of the aqueous humor, and has a high effect on reducing the intra-ocular pressure at night, especially for a patient intolerant to or having a poor effect with other medicine for reducing the intra-ocular pressure, and drugs commonly used in clinic are latanoprost, bimatoprost, travoprost, tafluprost, etc.

Glaucoma is a chronic eye disease, and the patient needs long-term medication. However, the unilateral preparations cannot usually effectively control the intra-ocular pressure, and two types of drugs need to be combined for use. For use of two types of unilateral preparations in combination or a dual compound preparation currently on the market (such as Combigan®, Cosopt®, etc.), due to increase of administration frequency, an exposed quantity of a preservative may also increase remarkably, which leads to poor safety, many side effects, poor patient compliance and other problems. Based on this, in the present disclosure, the β-adrenergic receptor blocker (timolol free alkali or salts of the same, carteolol free alkali or salts of the same, betaxolol free alkali or salts of the same, or metipranolol free alkali or salts of the same) and netarsudil representing the Rho kinase inhibitor are combined into a fixed dual compound medicine composition, which has the following strengths: (1) the intra-ocular pressure may be reduced through different action mechanisms, and the therapeutic effect is significant and exact; and (2) drug administration is performed once a day, the administration frequency is reduced, the exposed quantity of the preservative is reduced, occurrence of the side effects on the eyes is reduced, and patient compliance is improved remarkably. Thus, the ophthalmic medicine composition of the present disclosure provides possibility of reducing the intra-ocular pressure safely and effectively in clinic and treating glaucoma.

It may be known, in combination with prescribing information and related literature reports of commercially available netarsudil eye drops (Rhopress®), that netarsudil dimesylate is unstable and prone to generating precipitate in a boric acid-sodium borate buffer solution with pH higher than 5.4. Meanwhile, netarsudil dimesylate with the commercially available dose is in a solution state and good in stability in the boric acid-sodium borate buffer solution with pH lower than 5.4. Thus, in combination with eyes' tolerance to pH, in vitro stability of netarsudil dimesylate and a pH value range regulated by the netarsudil eye drops (Rhopress®), the ophthalmic medicine composition of the present disclosure is preliminarily determined as a boric acid-sodium borate buffer solution with a pH value in a range from 4.5 to 5.4. It is discovered unexpectedly by the applicant in a preliminary study that netarsudil dimesylate with the commercially available dose and the β-adrenergic receptor blocker (timolol maleate, carteolol hydrochloride, betaxolol hydrochloride and metipranolol hydrochloride) are unstable in the boric acid-sodium borate buffer solution with a pH value in a range from 4.5 to 5.4, namely, netarsudil dimesylate may be precipitated, and white precipitate appears in the solution. To verify the reason of causing precipitation of netarsudil dimesylate, maleic acid and methanesulfonic acid, hydrochloric acid and methanesulfonic acid, maleic acid and netarsudil dimesylate, hydrochloric acid and netarsudil dimesylate, timolol maleate and methanesulfonic acid, carteolol hydrochloride and methanesulfonic acid, betaxolol hydrochloride and methanesulfonic acid, metipranolol hydrochloride and methanesulfonic acid, timolol maleate and netarsudil dimesylate, carteolol hydrochloride and netarsudil dimesylate, betaxolol hydrochloride and netarsudil dimesylate, metipranolol hydrochloride and netarsudil dimesylate with the commercially available doses are respectively mixed in the boric acid-sodium borate buffer solution with a pH value in a range from 4.5 to 5.4, and a result shows that netarsudil dimesylate is incompatible with the maleic acid, the hydrochloric acid, timolol maleate, carteolol hydrochloride, betaxolol hydrochloride and metipranolol hydrochloride, and white precipitate may be generated. Thus it is indicated that netarsudil dimesylate is incompatible with the maleic acid and the hydrochloric acid.

Based on this, in the present disclosure, compatibility of the β-adrenergic receptor blocker (timolol maleate, carteolol hydrochloride, betaxolol hydrochloride, or metipranolol hydrochloride) with netarsudil dimesylate is improved through a salt engineering strategy, namely, by regulating salt-forming types of the β-adrenergic receptor blocker (timolol, carteolol, betaxolol or metipranolol) and the netarsudil, the compatibility of the β-adrenergic receptor blocker with the netarsudil in a water-for-injection solution with a pH value in a range from 4.5 to 5.4 is good. Meanwhile, the present disclosure constructs a triple compound preparation based on the netarsudil and the β-adrenergic receptor blocker, such as the netarsudil, the β-adrenergic receptor blocker (timolol, carteolol, betaxolol or metipranolol) and the prostaglandin analog. The triple compound preparation has strengths of a high effect on reducing the intra-ocular pressure, allowing drug administration once a day, etc., and it is expected to solve problems in only one existing triple compound preparation (Krytantek Ofteno®, dorzolamide 2%/timolol 0.5%/brimonidine 0.2%) on the global market that there is a serious adverse event, the effect on reducing the intra-ocular pressure is limited, etc.

### SUMMARY OF THE INVENTION

For overcoming defects of a poor therapeutic effect, many side effects, poor patient compliance and the like in an existing unilateral preparation, dual compound preparation and triple compound preparation for treating glaucoma, the present disclosure provides a dual compound medicine composition including both β-adrenergic receptor blocker and netarsudil; and a triple compound medicine composition including the β-adrenergic receptor blocker, the netarsudil and a prostaglandin analog, the problem that the β-adrenergic receptor blocker (timolol free alkali or salts of the same, carteolol free alkali or salts of the same, betaxolol free alkali or salfts of the same, or metipranolol free alkali or salts of the same) is incompatible with some types of salts of the netarsudil is solved successfully through a salt engineering strategy, and the compound medicine composition of the present disclosure can reduce an intra-ocular pressure more remarkably compared with a commercially available compound ophthalmic preparation.

That is, the present disclosure involves the the following content.
(1) In an aspect, the present disclosure provides an ophthalmic dual compound composition solution, including netarsudil free alkali or pharmaceutically acceptable salts of the same and a β-adrenergic receptor blocker (timolol free alkali or salts of the same, carteolol free alkali or salts of the same, betaxolol free alkali or salts of the same, or metipranolol free alkali or salts of the same) as active medicine components as well as a certain quantity of buffering agent, tonicity agent, preservative and pH regulator; and an ophthalmic triple compound composition solution, including netarsudil free alkali or pharmaceutically acceptable salts of the same, the β-adrenergic receptor blocker (timolol free alkali or salts of the same, carteolol free alkali or salts of the same, betaxolol free alkali or salts of the same, or metipranolol free alkali or salts of the same) and a prostaglandin analog as active medicine components as well as a certain quantity of buffering agent, tonicity agent, preservative and pH regulator.
(2) According to the above composition solution described in (1), the netarsudil is in a form of free alkali or any pharmaceutically acceptable salt (except mesylate) of the same in a case that the β-adrenergic receptor blocker is selected from timolol maleate, or carteolol hydrochloride, or betaxolol hydrochloride, or metipranolol hydrochloride, preferably, netarsudil maleate, netarsudil sulfate, netarsudil dihydrobromide, netarsudil dihydrochloride, netarsudil diformate, netarsudil dinitrate, netarsudil diacetate, netarsudil dibenzoate, netarsudil diphenylacetate, netarsudil succinate, netarsudil oxalate, netarsudil dihydriodate, or netarsudil dipropionate.
(3) According to the above composition solution described in (1), the timolol is in a form of free alkali or any pharmaceutically acceptable salt (except maleate) of the same in a case that the netarsudil is dimesylate, preferably, timolol mesylate, timolol sulfate, timolol hydrobromide, timolol phosphate, timolol nitrate, timolol citrate, timolol tartrate, timolol salicylate, timolol malate, timolol lactate, timolol phenylacetate, timolol succinate, timolol hydriodate, timolol formate, timolol acetate, timolol benzoate, timolol esilate, timolol oxalate or timolol propionate; the carteolol is in a form of free alkali or any pharmaceutically acceptable salt (except hydrochloride) of the same, preferably, carteolol mesylate, carteolol hydrobromide, carteolol sulfate, carteolol esilate, carteolol nitrate, carteolol citrate, carteolol tartrate, carteolol salicylate, carteolol malate, carteolol lactate, carteolol phenylacetate, carteolol succinate, carteolol hydriodate, carteolol formate, carteolol acetate, carteolol benzoate, carteolol esilate, carteolol oxalate or carteolol propionate; the betaxolol is in a form of free alkali or any pharmaceutically acceptable salt (except hydrochloride) of the same, preferably, betaxolol mesylate, betaxolol hydrobromide, betaxolol sulfate, betaxolol esilate, betaxolol nitrate, betaxolol citrate, betaxolol tartrate, betaxolol salicylate, betaxolol malate, betaxolol lactate, betaxolol phenylacetate, betaxolol succinate, betaxolol hydriodate, betaxolol formate, betaxolol acetate, betaxolol benzoate, betaxolol esilate, betaxolol oxalate or betaxolol propionate; and the metipranolol is in a form of free alkali or any pharmaceutically acceptable salt (except hydrochloride) of the same, preferably, metipranolol mesylate, metipranolol hydrobromide, metipranolol sulfate, metipranolol esilate, metipranolol nitrate, metipranolol citrate, metipranolol tartrate, metipranolol salicylate, metipranolol malate, metipranolol lactate, metipranolol phenylacetate, metipranolol succinate, metipranolol hydriodate, metipranolol formate, metipranolol acetate, metipranolol benzoate, metipranolol esilate, metipranolol oxalate or metipranolol propionate.
(4) According to the above composition solution described in (1), the prostaglandin analog includes but is not limited to latanoprost, bimatoprost, travoprost, tafluprost, AR-102, cloprostenol isopropyl ester, 13,14-dihydrocloprostenol isopropyl ester, latanoprostene bunod, unoprostone, PGF1α isopropyl ester, PGF2α isopropyl ester, PGF3α isopropyl ester and fluprostenol isopropyl ester.
(5) According to the above composition solution described in (1), the tonicity agent includes but is not limited to glycerol, sorbitol, mannitol, propylene glycol, erythritol, arabitol, xylitol, ribitol, galactitol, polyethylene glycol, lactitol and other sugar alcohol, sodium chloride, potassium chloride and calcium chloride, or any combination of the above.
(6) According to the above composition solution described in (1), the buffering agent includes but is not limited to boric acid or salts of the same, sodium dihydrogen phosphate dihydrate, sodium dihydrogen phosphate monohydrate, sodium phosphate anhydrate, citric acid or salts of the same, gluconic acid or salts of the same, acetic acid or salts of the same, phosphoric acid or salts of the same, various amino acids such as glutamic acid and s-aminocaproic acid, a tris(hydroxymethyl) aminomethane buffer, or any combination of the above.
(7) According to the above composition solution described in (1), the preservative includes but is not limited to benzalkonium chloride, thimerosal, chlorbutanol, methyl p-hydroxybenzoate, propyl p-hydroxybenzoate, phenethyl alcohol, edetate disodium, boric acid, sorbic acid or any combination of the above.
(8) According to the above composition solution described in (1), examples of the pH regulator include but are not limited to sodium hydroxide, potassium hydroxide, sodium carbonate, sodium hydrogen carbonate, hydrochloric acid, citric acid or salts of the same, phosphoric acid or salts of the same, acetic acid or salts of the same, and tartaric acid or salts of the same.
(9) According to the above composition solution described in (1) to (8), the composition includes 0.02% w/v to 4.0% w/v β-adrenergic receptor blocker (timolol free alkali or salts of the same, carteolol free alkali or salts of the same, betaxolol free alkali or salts of the same, or metipranolol free alkali or salts of the same).
(10) According to the above composition solution described in (1) to (8), the composition includes 0.005% w/v to 0.10% w/v netarsudil free alkali or salts of the same.
(11) According to the above composition solution described in (1) to (8), the composition includes 0.0005% w/v to 0.05% w/v prostaglandin analog.
(12) According to the above composition solution described in (1) to (8), the composition includes 1.0% w/v to 10.0% w/v mannitol.
(13) According to the above composition solution described in (1) to (8), the composition includes 0.05% w/v boric acid.
(14) According to the above composition solution described in (1) to (8), the composition includes 0.001% w/v to 0.02% w/v benzalkonium chloride.
(15) The above composition solution described in (1) to (14) has an osmotic pressure in a range from 280 mOsmol/kg to 320 mOsmol/kg.
(16) The above composition solution described in (1) to (15) has pH in a range from 4.5 to 5.4.
(17) According to the above composition solution described in (1) to (16), compared with zero day, a content of each main medicine component has no significant change compared with zero day after being stored at 5° C. for 24 months, which remains consistent with a storage condition of commercially available eye drops before opening.
(18) According to the above composition solution described in (1) to (16), compared with zero day, a content of each main medicine component has no significant change compared with zero day after being stored at 25° C. for 6 weeks, which meets a requirement for a storage condition of commercially available eye drops after opening.
(19) According to the above composition solution described in (1) to (16), compared with zero day, a content of each main medicine component has no significant change compared with zero day after being stored at 40° C. for 14 days, which meets a requirement for departing from a storage condition of commercially available eye drops for a short time.
(20) In another aspect, the present disclosure provides use of the composition solution in the first aspect in preparation for medicine for preventing or treating an eye disease.
(21) According to the use described in (20), the eye disease is glaucoma or symptoms related to the same.
(22) In a third aspect, the present disclosure provides use of the composition solution in the first aspect in preparation of medicine for reducing an intra-ocular pressure.

Compared with the prior art, the present disclosure has the following beneficial effects.
(1) Through a salt engineering strategy, a problem of incompatibility of the β-adrenergic receptor blocker (timolol free alkali or salts of the same, carteolol free alkali or salts of the same, betaxolol free alkali or salts of the same, or metipranolol free alkali or salts of the same) with some types of salts of netarsudil is solved, so as to obtain the solution having the excellent stability and including the β-adrenergic receptor blocker (timolol free alkali or salts of the same, carteolol free alkali or salts of the same, betaxolol free alkali or salts of the same, or metipranolol free alkali or salts of the same) and the netarsudil.
(2) In another aspect, the medicine composition solution provided by the present disclosure is useful for preparing medicine for preventing or treating the eye disease (especially, glaucoma or symptoms related to the same) and medicine for reducing the intra-ocular pressure, and the medicine composition of the present disclosure can reduce the intra-ocular pressure more remarkably compared with commercially available compound ophthalmic preparations.

### BRIEF DESCRIPTION OF DRAWINGS

FIG. 1 represents a dosage regimen of an ophthalmic medicine composition solution in Embodiments 1 to 10, a positive control drug treatment group 1 (Rhopress®, netarsudil mesylate eye drops), and a positive control drug treatment group 2 (Mikelan®, carteolol hydrochloride eye drops).

FIG. 2 represents a column diagram made according to data of an intra-ocular pressure variation value (2A) and an intra-ocular pressure actual measured value (2B) of each group of rabbits measured on the fifteenth day and the twentieth day when an ophthalmic medicine composition solution of Embodiments 1 to 10 and positive control drugs (Rhopress® and Mikelan®) are administered once a day for 10 days on end starting with the tenth day. On the twentieth day: &: P<0.05, a treatment group 1 and a treatment group 2 compared with Embodiment groups 1 to 10; #: P<0.05, Embodiment groups 1, 2 and 3 compared with Embodiment groups 4 and 5; *: P<0.05, Embodiment groups 6, 7 and 8 compared with Embodiment groups 9 and 10; and n.s: P>0.05, Embodiment groups 4 and 5 compared with Embodiment groups 9 and 10.

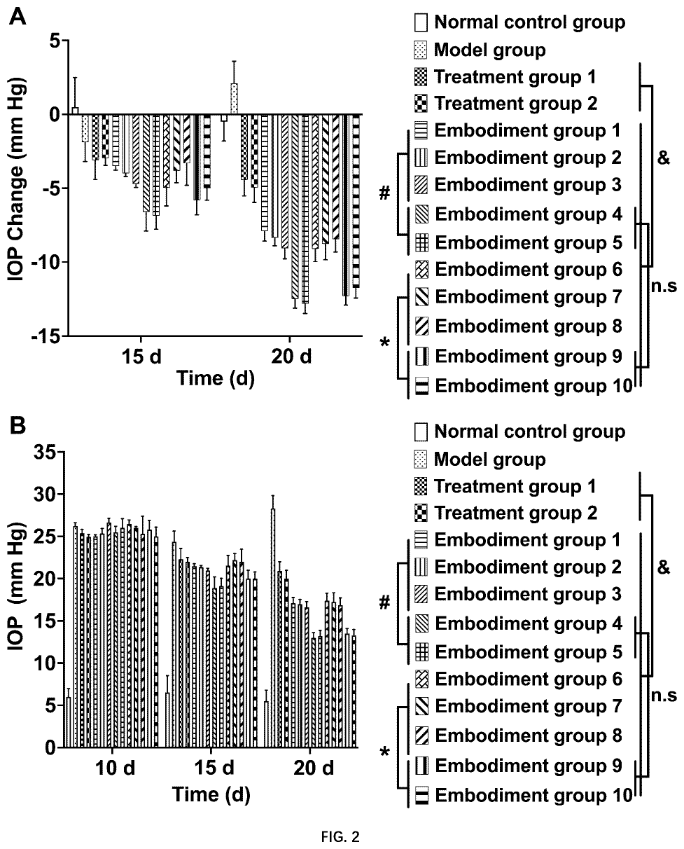

FIG. 3 represents a dosage regimen of an ophthalmic medicine composition solution in Embodiments 12 to 21, a positive control drug treatment group 1 and a positive control drug treatment group 3 (Timoptic®, timolol maleate eye drops).

FIG. 4 represents a column diagram made according to data of an intra-ocular pressure variation value (4A) and an intra-ocular pressure actual measured value (4B) of each group of rabbits measured on the fifteenth day and the twentieth day when an ophthalmic medicine composition solution of Embodiments 12 to 21 and a positive control drug (Timoptic® and Rhopress®) are administered once a day for 10 days on end starting with the tenth day. On the twentieth day: &: P<0.05, a treatment group 1 and a treatment group 3 compared with Embodiment groups 12 to 21; #: P<0.05, Embodiment groups 12, 13 and 14 compared with Embodiment groups 15 and 16; *: P<0.05, Embodiment groups 17, 18 and 19 compared with Embodiment groups 20 and 21; and n.s: P>0.05, Embodiment groups 15 and 16 compared with Embodiment groups 20 and 21.

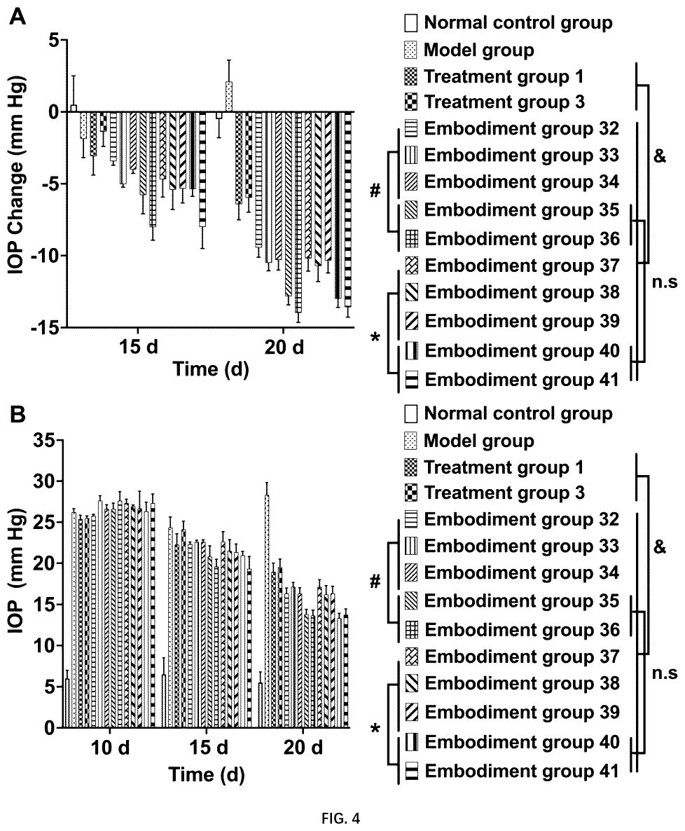

FIG. 5 represents a dosage regimen of an ophthalmic medicine composition solution in Embodiments 83 to 92, a positive control drug treatment group 4 (Xalacom®, latanoprost and timolol eye drops), and a positive control drug treatment group 5 (Rocklatan®, latanoprost and netarsudil eye drops).

FIG. 6 represents a column diagram made according to data of an intra-ocular pressure variation value (6A) and an intra-ocular pressure actual measured value (6B) of each group of rabbits measured on the fifteenth day and the twentieth day when an ophthalmic medicine composition solution of Embodiments 83 to 92 and positive control drugs (Xalacom® and Rocklatan®) are administered once a day for 10 days on end starting with the tenth day. On the twentieth day: &: P<0.05, a treatment group 4 and a treatment group 5 compared with Embodiment groups 83 to 92; #: P<0.05, Embodiment groups 83, 84 and 85 compared with Embodiment groups 86 and 87; *: P<0.05, Embodiment groups 88, 89 and 90 compared with Embodiment groups 91 and 92; and n.s: P>0.05, Embodiment groups 86 and 87 compared with Embodiment groups 91 and 92.

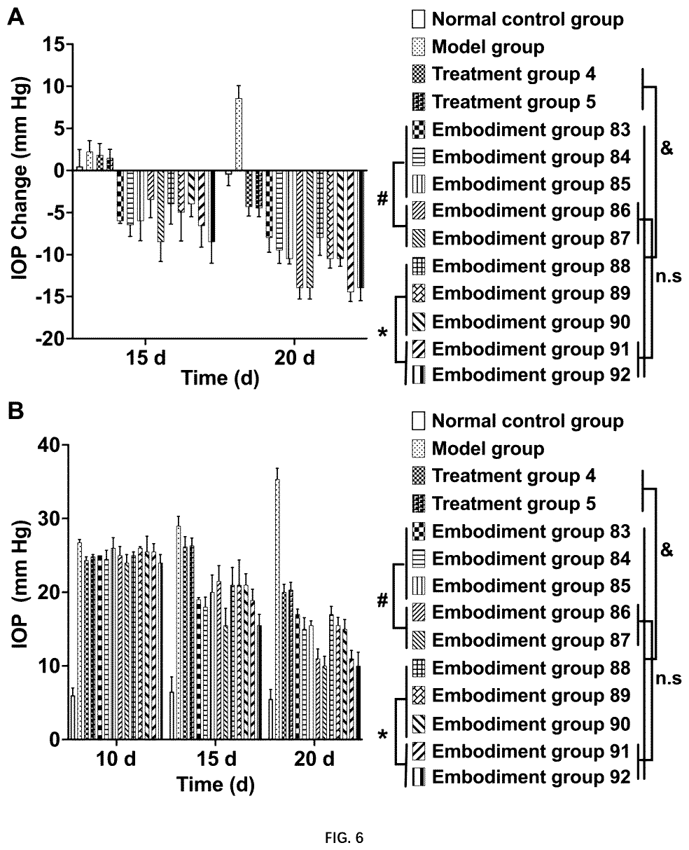

FIG. 7 represents a dosage regimen of an ophthalmic medicine composition solution in Embodiments 94 to 103 and a positive control drug treatment group 6 (Ganfort®, bimatoprost and timolol maleate eye drops).

FIG. 8 represents a column diagram made according to data of an intra-ocular pressure variation value (8A) and an intra-ocular pressure actual measured value (8B) of each group of rabbits measured on the fifteenth day and the twentieth day when an ophthalmic medicine composition solution of Embodiments 94 to 103 and a positive control drug (Ganfort®) are administered once a day for 10 days on end starting with the tenth day. On the twentieth day: &: P<0.05, a treatment group 6 compared with Embodiment groups 94 to 103; #: P<0.05, Embodiment groups 94, 95 and 96 compared with Embodiment groups 97 and 98; *: P<0.05, Embodiment groups 99, 100 and 101 compared with Embodiment groups 102 and 103; and n.s: P>0.05, Embodiment groups 97 and 98 compared with Embodiment groups 102 and 103.

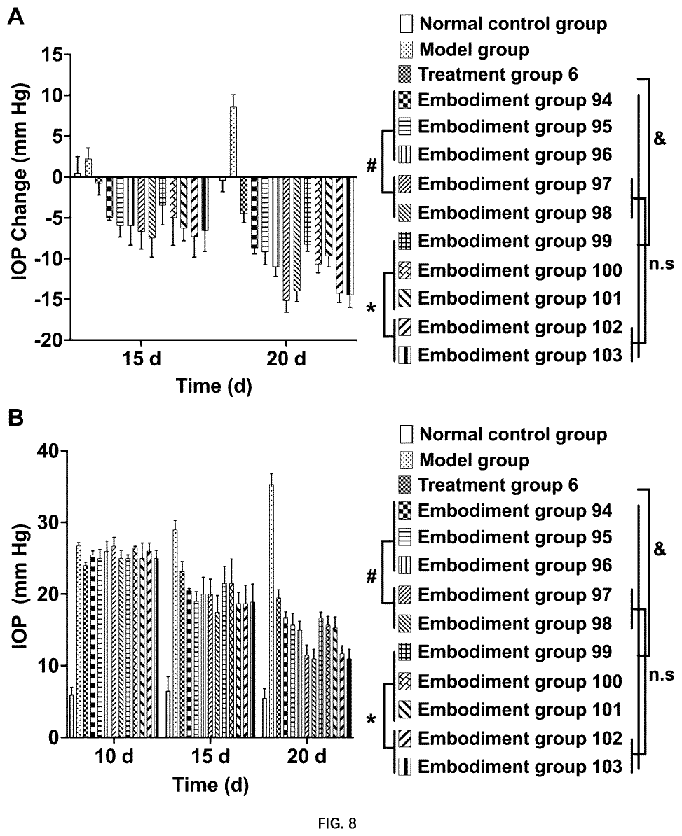

FIG. 9 represents a dosage regimen of an ophthalmic medicine composition solution in Embodiments 105 to 114 and a positive control drug treatment group 7 (DuoTray®, travoprost and timolol maleate eye drops).

FIG. 10 represents a column diagram made according to data of an intra-ocular pressure variation value (10A) and an intra-ocular pressure actual measured value (10B) of each group of rabbits measured on the fifteenth day and the twentieth day when an ophthalmic medicine composition solution of Embodiments 105 to 114 and a positive control drug (DuoTrav®) are administered once a day for 10 days on end starting with the tenth day. On the twentieth day: &: P<0.05, a treatment group 7 compared with Embodiment groups 105 to 114; #: P<0.05, Embodiment groups 105, 106 and 107 compared with Embodiment groups 108 and 109; *: P<0.05, Embodiment groups 110, 111 and 112 compared with Embodiment groups 113 and 114; and n.s: P>0.05, Embodiment groups 108 and 109 compared with Embodiment groups 113 and 114.

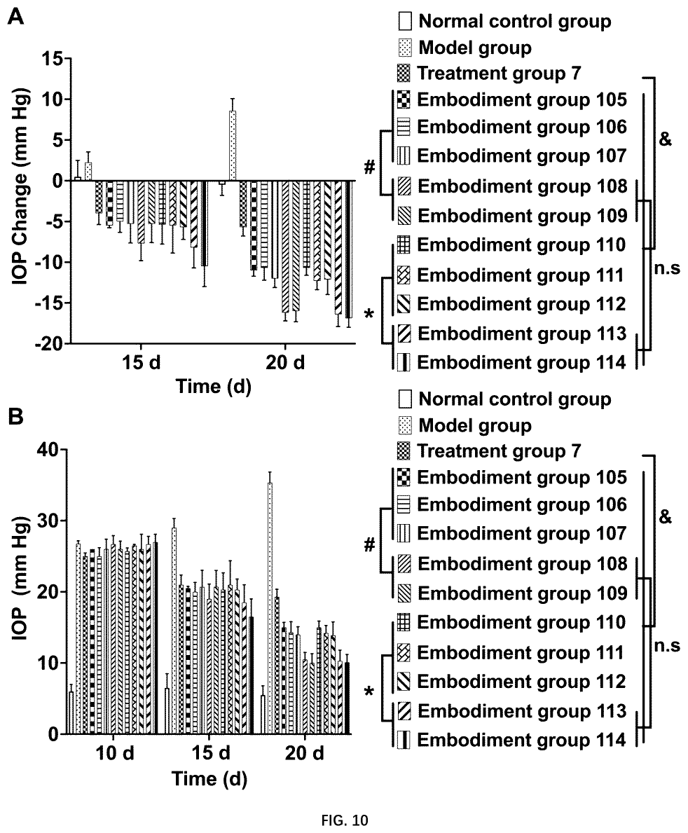

FIG. 11 represents a dosage regimen of an ophthalmic medicine composition solution in Embodiments 116 to 125 and a positive control drug treatment group 8 (Tapcom®, tafluprost timolol maleate eye drops).

FIG. 12 represents a column diagram made according to data of an intra-ocular pressure variation value (12A) and an intra-ocular pressure actual measured value (12B) of each group of rabbits measured on the fifteenth day and the twentieth day when an ophthalmic medicine composition solution of Embodiments 116 to 125 and a positive control drug (Tapcom®) are administered once a day for 10 days on end starting with the tenth day. On the twentieth day: &: P<0.05, a treatment group 8 compared with Embodiment groups 116 to 125; #: P<0.05, Embodiment groups 116, 117 and 118 compared with Embodiment groups 119 and 120; *: P<0.05, Embodiment groups 121, 122 and 123 compared with Embodiment groups 124 and 125; and n.s: P>0.05, Embodiment groups 119 and 120 compared with Embodiment groups 124 and 125.

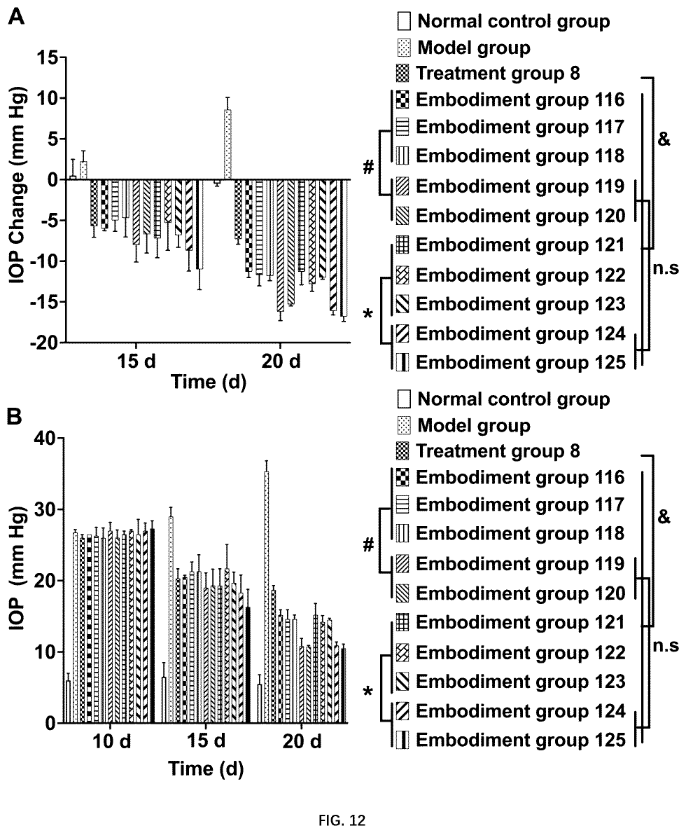

FIG. 13 represents a dosage regimen of an ophthalmic medicine composition solution in Embodiments 147 to 156, a positive control drug treatment group 5, and a positive control drug treatment group 9 (Mikeluna®, carteolol hydrochloride and latanoprost eye drops).

FIG. 14 represents a column diagram made according to data of an intra-ocular pressure variation value (14A) and an intra-ocular pressure actual measured value (14B) of each group of rabbits measured on the fifteenth day and the twentieth day when an ophthalmic medicine composition solution of Embodiments 147 to 156 and positive control drugs (Rocklatan® and Mikeluna®) are administered once a day for 10 days on end starting with the tenth day. On the twentieth day: &: P<0.05, a treatment group 5 and a treatment group 9 compared with Embodiment groups 147 to 156; #: P<0.05, Embodiment groups 147, 148 and 149 compared with Embodiment groups 150 and 151; *: P<0.05, Embodiment groups 152, 153 and 154 compared with Embodiment groups 155 and 156; and n.s: P>0.05, Embodiment groups 150 and 151 compared with Embodiment groups 155 and 156.

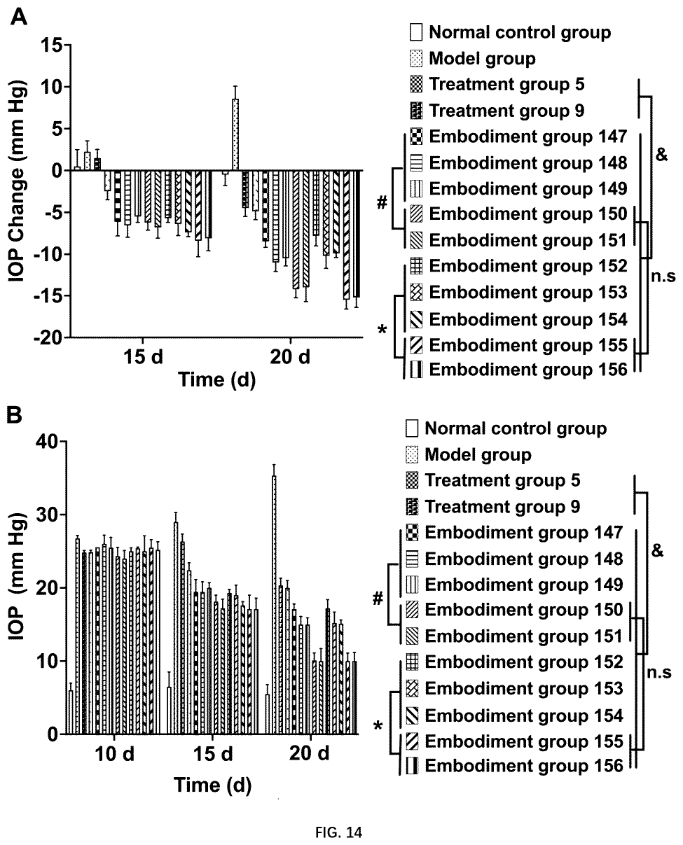

FIG. 15 represents a dosage regimen of an ophthalmic medicine composition solution in Embodiments 158 to 167, a positive control drug treatment group 5 and a positive control drug treatment group 6.

FIG. 16 represents a column diagram made according to data of an intra-ocular pressure variation value (16A) and an intra-ocular pressure actual measured value (16B) of each group of rabbits measured on the fifteenth day and the twentieth day when an ophthalmic medicine composition solution of Embodiments 158 to 167 and positive control drugs (Rocklatan® and Ganfort®) are administered once a day for 10 days on end starting with the tenth day. On the twentieth day: &: P<0.05, a treatment group 5 and a treatment group 6 compared with Embodiment groups 158 to 167; #: P<0.05, Embodiment groups 158, 159 and 160 compared with Embodiment groups 161 and 162; *: P<0.05, Embodiment groups 163, 164 and 165 compared with Embodiment groups 166 and 167; and n.s: P>0.05, Embodiment groups 161 and 162 compared with Embodiment groups 166 and 167.

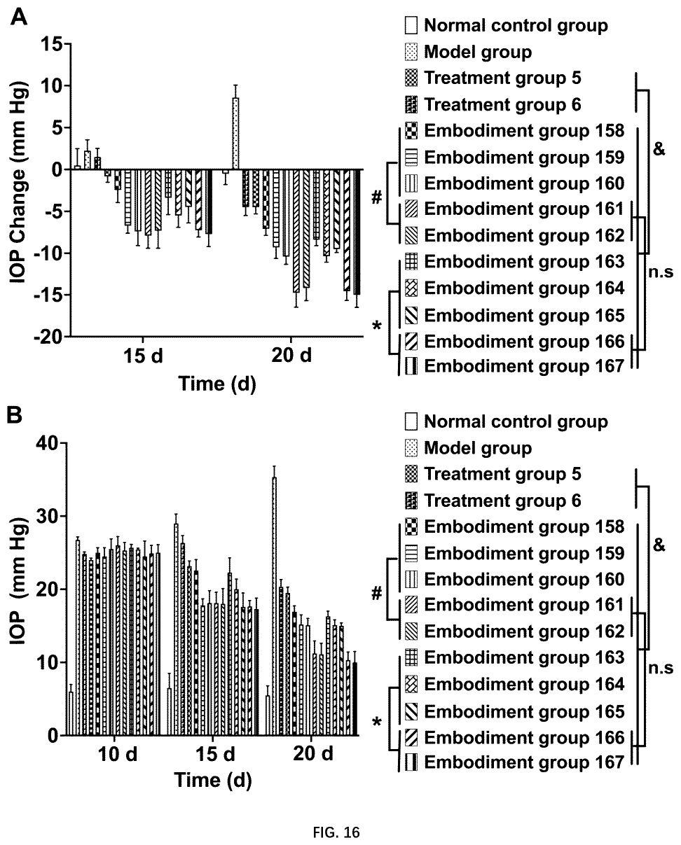

FIG. 17 represents a dosage regimen of an ophthalmic medicine composition solution in Embodiments 169 to 178, a positive control drug treatment group 5 and a positive control drug treatment group 7.

FIG. 18 represents a column diagram made according to data of an intra-ocular pressure variation value (18A) and an intra-ocular pressure actual measured value (18B) of each group of rabbits measured on the fifteenth day and the twentieth day when an ophthalmic medicine composition solution of Embodiments 169 to 178 and positive control drugs (Rocklatan® and DuoTrav®) are administered once a day for 10 days on end starting with the tenth day. On the twentieth day: &: P<0.05, a treatment group 5 and a treatment group 7 compared with Embodiment groups 169 to 178; #: P<0.05, Embodiment groups 169, 170 and 171 compared with Embodiment groups 172 and 173; *: P<0.05, Embodiment groups 174, 175 and 176 compared with Embodiment groups 177 and 178; and n.s: P>0.05, Embodiment groups 172 and 173 compared with Embodiment groups 177 and 178.

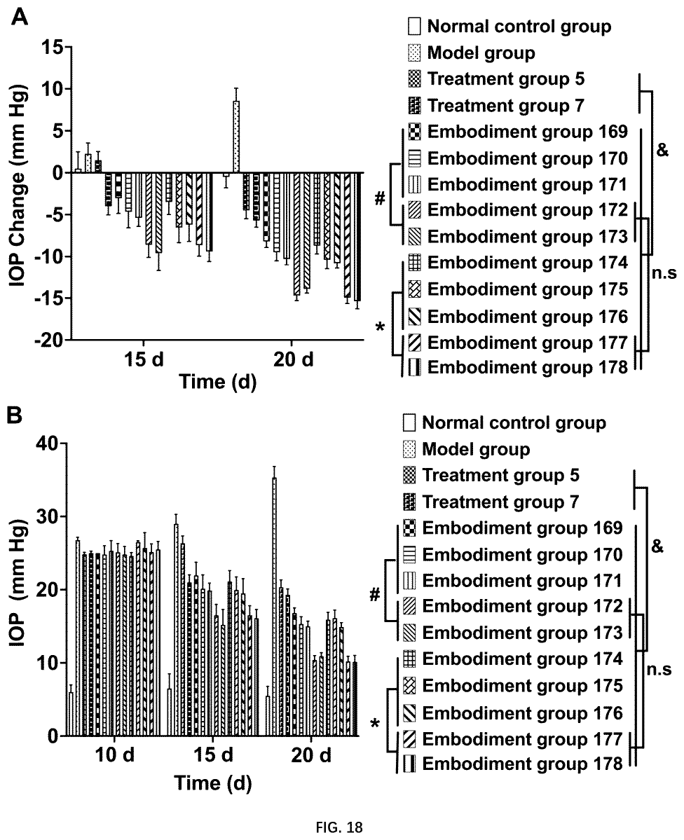

FIG. 19 represents a dosage regimen of an ophthalmic medicine composition solution in Embodiments 180 to 189, a positive control drug treatment group 5 and a positive control drug treatment group 8.

FIG. 20 represents a column diagram made according to data of an intra-ocular pressure variation value (20A) and an intra-ocular pressure actual measured value (20B) of each group of rabbits measured on the fifteenth day and the twentieth day when an ophthalmic medicine composition solution of Embodiments 180 to 189 and positive control drugs (Rocklatan® and Tapcom®) are administered once a day for 10 days on end starting with the tenth day. On the twentieth day: &: P<0.05, a treatment group 5 and a treatment group 8 compared with Embodiment groups 180 to 189; #: P<0.05, Embodiment groups 180, 181 and 182 compared with Embodiment groups 183 and 184; *: P<0.05, Embodiment groups 185, 186 and 187 compared with Embodiment groups 188 and 189; and n.s: P>0.05, Embodiment groups 183 and 184 compared with Embodiment groups 188 and 189.

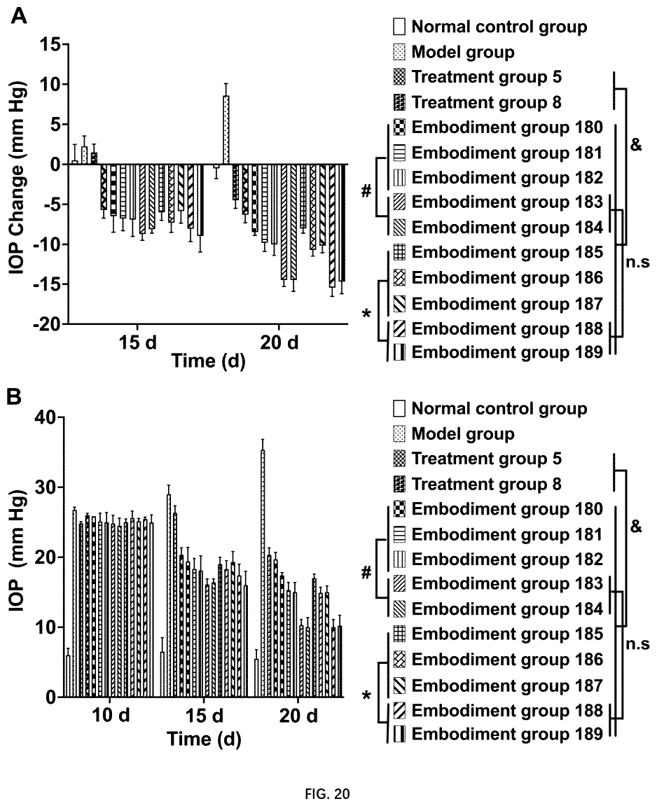

### DETAILED DESCRIPTION OF THE INVENTION

In an ophthalmic medicine composition of the present disclosure, when netarsudil is dimesylate, timolol is preferably in a salt form formed from a pharmaceutically acceptable acid (except maleic acid). As specific acid, methanesulfonic acid, sulfuric acid, hydrobromic acid, phosphoric acid, nitric acid, citric acid, tartaric acid, salicylic acid, malic acid, ethanesulfonic acid, lactic acid, or phenylacetic acid may be listed, more preferably methanesulfonic acid.

In the ophthalmic medicine composition of the present disclosure, when the netarsudil is dimesylate, carteolol is preferably in a salt form formed from a pharmaceutically acceptable acid (except hydrochloric acid). As specific acid, methanesulfonic acid, sulfuric acid, hydrobromic acid, phosphoric acid, nitric acid, citric acid, tartaric acid, salicylic acid, malic acid, ethanesulfonic acid, lactic acid, or phenylacetic acid may be listed, more preferably methanesulfonic acid.

In the ophthalmic medicine composition of the present disclosure, when the netarsudil is dimesylate, betaxolol is preferably in a salt form formed from a pharmaceutically acceptable acid (except hydrochloric acid). As specific acid, methanesulfonic acid, sulfuric acid, hydrobromic acid, phosphoric acid, nitric acid, citric acid, tartaric acid, salicylic acid, malic acid, ethanesulfonic acid, lactic acid, or phenylacetic acid may be listed, more preferably methanesulfonic acid.

In the ophthalmic medicine composition of the present disclosure, when the netarsudil is dimesylate, metipranolol is preferably in a salt form formed from a pharmaceutically acceptable acid (except hydrochloric acid). As specific acid, methanesulfonic acid, sulfuric acid, hydrobromic acid, phosphoric acid, nitric acid, citric acid, tartaric acid, salicylic acid, malic acid, ethanesulfonic acid, lactic acid, or phenylacetic acid may be listed, more preferably methanesulfonic acid.

In an ophthalmic medicine composition solution of the present disclosure, when the timolol is maleate, the carteolol is hydrochloride, the betaxolol is hydrochloride, or the metipranolol is hydrochloride, the netarsudil is preferably in a salt form formed from a pharmaceutically acceptable acid (except methanesulfonic acid). As specific acid, maleic acid, sulfuric acid, hydrobromic acid, hydrochloric acid, formic acid, nitric acid, acetic acid, phenylacetic acid, butanedioic acid, oxalic acid, hydroiodic acid, or propionic acid may be listed, more preferably maleic acid and hydrochloric acid.

In the medicine composition of the present disclosure, there is no particular limit as long as salts of the timolol, the carteolol, the betaxolol, the metipranolol and the netarsudil are pharmacologically permissible salts. Specifically, examples of appropriate inorganic anions include but are not limited to inorganic anions derived from the following inorganic acid: hydrochloric acid, hydrobromic acid, hydroiodic acid, sulfuric acid, sulphurous acid, nitric acid, nitrous acid, phosphoric acid and phosphorous acid. Examples of appropriate organic anions include but are not limited to organic anions derived from the following organic acid: 2-(acetoxy)benzoic acid, acetic acid, ascorbic acid, aspartic acid, benzoic acid, camphorsulfonic acid, cinnamic acid, citric acid, edetic acid, ethanedisulfonic acid, ethanesulfonic acid, fumaric acid, glucoheptonic acid, gluconic acid, glutamic acid, glycolic acid hydroxymaleic acid, hydroxynaphthalene carboxylic acid, isethionic acid, lactic acid, lactobionic acid, lauric acid, maleic acid, malic acid, ethanesulfonic acid, methanesulfonic acid, galactaric acid, oleic acid, oxalic acid, palmitic acid, pamoic acid, pantothenic acid, phenylacetic acid, sulfonic acid, propionic acid, pyruvic acid, salicylic acid, stearic acid, succinic acid, sulfanilic acid, tartaric acid, p-toluenesulfonic acid, and valeric acid. Examples of appropriate polymer organic anions include but are not limited to polymer organic anions derived from the following polyacid: tannic acid and carboxymethylcellulose.

The medicine composition of the present disclosure further includes at least one type of prostaglandin analog as an active compound. Specifically, examples of the prostaglandin analog include but are not limited to prostaglandin of latanoprost, bimatoprost, travoprost, tafluprost, AR-102, cloprostenol isopropyl ester, 13,14-dihydrocloprostenol isopropyl ester, latanoprostene bunod, unoprostone, PGF1α isopropyl ester, PGF2α isopropyl ester, PGF3α isopropyl ester and fluprostenol isopropyl ester.

In the ophthalmic medicine composition solution of the present disclosure, the β-adrenergic receptor blocker (timolol free alkali or salts of the same, carteolol free alkali or salts of the same, betaxolol free alkali or salts of the same, or metipranolol free alkali or salts of the same) has a concentration in a range from 0.02% w/v to 4.0% w/v, more preferably 0.3% w/v to 2.0% w/v.

In the ophthalmic medicine composition solution of the present disclosure, the netarsudil free alkali or salts of the same has/have a concentration in a range from 0.005% w/v to 0.1% w/v, more preferably 0.02% w/v to 0.04% w/v, most preferably 0.02% w/v.

In the ophthalmic medicine composition solution of the present disclosure, the prostaglandin analog has a concentration in a range from 0.0005% w/v to 0.05% w/v, more preferably 0.0015% w/v to 0.03% w/v.

The ophthalmic medicine composition solution of the present disclosure may include a tonicity agent. Examples of the tonicity agent include but are not limited to sodium chloride, potassium chloride, mannitol, dextrose, glycerol and propylene glycol. Appropriately, the tonicity agent may exist in the ophthalmic composition in an amount of about 0.01% w/v to about 10% w/v, or about 1% w/v to about 10% w/v, or about 2.5% w/v to about 7.5% ow/v, or about 4% w/v to about 6% w/v. Appropriately, the tonicity agent may exist in an amount of at least 0.01% w/v, at least 0.05% w/v, at least 1% w/v, at least 2.5% w/v, at least 4% w/v, or at least 5% w/v. Appropriately, the tonicity agent may exist in an amount not exceeding 10% w/v, not exceeding 8% w/v, or not exceeding 6% w/v. In an implementation, the tonicity agent may exist in the ophthalmic composition in an amount of about 3.0% w/v, about 3.1% w/v, about 3.2% w/v, about 3.3% w/v, about 3.40% w/v, about 3.5% w/v, about 3.6% w/v, about 3.7% w/v, about 3.8% w/v, about 3.9% w/v, about 4.0% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5.0% w/v, about 5.1% w/v, about 5.2% w/v, about 5.3% w/v, about 5.4% w/v or about 5.5% w/v. The tonicity agent may be appropriately mannitol.

The ophthalmic medicine composition solution of the present disclosure may include a buffering agent. The appropriate buffering agent includes but is not limited to acetic acid, citric acid, carbonic acid, phosphoric acid, boric acid, pharmaceutically acceptable salts of them, tromethamine, and a combination of them. The buffering agent may exist in the ophthalmic composition in an amount of about 0.01% w/v to about 10 w/v, or about 0.1% w/v to about 0.9% w/v, or about 0.3% ow/v to about 0.8% w/v, or about 0.40 w/v to about 0.6% w/v. Appropriately, the buffering agent may exist in an amount of at least 0.01% w/v, at least 0.05% ow/v, at least 0.1% w/v, at least 0.3% ow/v, or at least 0.5% ow/v. Appropriately, the buffering agent may exist in an amount not exceeding 1.0% w/v, not exceeding 0.8% w/v, or not exceeding 0.60% w/v. In an implementation, the buffering agent may exist in the ophthalmic composition in an amount of about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, or about 0.1% w/v. The buffering agent may be appropriately boric acid.

The ophthalmic medicine composition solution of the present disclosure may include a preservative. The appropriate preservative includes but is not limited to benzalkonium chloride, thimerosal, chlorbutanol, methyl p-hydroxybenzoate, propyl p-hydroxybenzoate, phenethyl alcohol, edetate disodium, boric acid, sorbic acid or another reagent known to those skilled in the art. The preservative may exist in the ophthalmic composition in an amount of about 0.001% w/v to about 0.02% w/v. The preservative may be appropriately benzalkonium chloride.

The ophthalmic medicine composition solution of the present disclosure may include a pH regulator, and an amount of the pH regulator is enough to regulate pH of the composition to a range from about 4 to about 9. Appropriately, the ophthalmic composition may have pH in a range from about 4.5 to about 5.4. The appropriate pH regulator includes but is not limited to sodium hydroxide.

### EMBODIMENTS

The present disclosure has a plurality of aspects described through the following non-restrictive embodiments. In various embodiments, the following materials and characterization technique are used.

### Embodiments 1 to 5: A Preparation Including Netarsudil Dimesylate and Carteolol Mesylate

TABLE 1
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 1 | | 2 | | 3 | | 4 | | 5 | |
| 1. | Netarsudil dimesylate | 14.2 | mg | 14.2 | mg | 28.5 | mg | 28.5 | mg | 57.0 | mg |
| 2. | Carteolol mesylate | 1329 | mg | 2658 | mg | 1329 | mg | 2658 | mg | 5316 | mg |
| 3. | Boric acid | 50 | mg | 50 | mg | 50 | mg | 50 | mg | 50 | mg |
| 4. | Mannitol | 3700 | mg | 3700 | mg | 3700 | mg | 3700 | mg | 3700 | mg |
| 5. | Benzalkonium chloride | 5 | mg | 5 | mg | 5 | mg | 5 | mg | 5 | mg |
| 6. | Sodium hydroxide | q.s. | | q.s. | | q.s. | | q.s. | | q.s. | |
| 7. | Water for injection | 100 | ml | 100 | ml | 100 | ml | 100 | ml | 100 | ml |
| | added by | | | | | | | | | | |

A preparation process is as follows: 1) 95% of a prescription dosage of water for injection is weighed, prescription dosages of active components and adjuvants are added and stirred to be dissolved completely, and pH is regulated with a sodium hydroxide solution (10%) to a range from 4.5 to 5.4; 2) water for injection is supplemented to a full prescription dosage, stirring is performed to be uniform, and a volume reaches 100%; and 3) the solution in step 2 is put in a low-density polyethylene medicinal eye drop bottle, and its stability is studied under different storage temperature conditions.

TABLE 2
Contents of various main medicine components in Embodiments
1 to 5 after being stored at 5° C. for 24 months

| | | Embodiment | Embodiment | Embodiment | Embodiment | Embodiment |
|---|---|---|---|---|---|---|
| | | 1 | 2 | 3 | 4 | 5 |
| No. | Component | Content % | | | | |
| 1. | Netarsudil | 99.3 | 98.6 | 97.4 | 99.1 | 99.0 |
| | dimesylate | | | | | |
| 2. | Carteolol | 99.7 | 99.2 | 99.3 | 99.0 | 99.1 |
| | mesylate | | | | | |

TABLE 3
Contents of various main medicine components in commercially
available eye drops (Mikelan ® and Rhopress ®) after
being stored at 5° C. for 24 months

| | |
|---|---|
| Mikelan ® | Rhopress ® |

| | | |
|---|---|---|
| No. | Component | Content % |

| | | | |
|---|---|---|---|
| 1. | Netarsudil dimesylate | — | 100.0 |
| 2. | Carteolol hydrochloride | 99.9 | — |

According to Embodiments 1 to 5, the preparation including netarsudil dimesylate and carteolol mesylate has good stability after being stored under a storage condition of 5° C. for 24 months, which meets a requirement for a storage condition of the commercially available eye drops (Mikelan® and Rhopress®) before opening.

TABLE 4
Contents of various main medicine components in Embodiments
1 to 5 after being stored at 25° C. for 6 weeks

| | | Embodiment | Embodiment | Embodiment | Embodiment | Embodiment |
|---|---|---|---|---|---|---|
| | | 1 | 2 | 3 | 4 | 5 |
| No. | Component | Content % | | | | |
| 1. | Netarsudil | 99.1 | 99.0 | 99.3 | 99.1 | 98.7 |
| | dimesylate | | | | | |
| 2. | Carteolol | 100.1 | 101.2 | 100.6 | 100.4 | 100.6 |
| | mesylate | | | | | |

TABLE 5
Contents of various main medicine components in commercially
available eye drops (Mikelan ® and Rhopress ®) after
being stored at 25° C. for 6 weeks

| | |
|---|---|
| Mikelan ® | Rhopress ® |

| | | |
|---|---|---|
| No. | Component | Content % |

| | | | |
|---|---|---|---|
| 1. | Netarsudil dimesylate | — | 99.9 |
| 2. | Carteolol hydrochloride | 99.9 | — |

According to Embodiments 1 to 5, the preparation including netarsudil dimesylate and carteolol mesylate has good stability after being stored under a storage condition of 25° C. for 6 weeks, which meets a requirement for a storage condition of the commercially available eye drops (Mikelan® and Rhopress®) after opening.

TABLE 6
Contents of various main medicine components in Embodiments
1 to 5 after being stored at 40° C. for 14 days

| | | Embodiment | Embodiment | Embodiment | Embodiment | Embodiment |
|---|---|---|---|---|---|---|
| | | 1 | 2 | 3 | 4 | 5 |
| No. | Component | Content % | | | | |
| 1. | Netarsudil | 98.9 | 99.5 | 99.0 | 99.1 | 99.9 |
| | dimesylate | | | | | |
| 2. | Carteolol | 99.9 | 100.0 | 101.3 | 100.5 | 100.3 |
| | mesylate | | | | | |

Table 7. Contents of various main medicine components in commercially available eye drops (Mikelan® and Rhopress®) after being stored at 40° C. for 14 days

TABLE 7
Contents of various main medicine components in commercially
available eye drops (Mikelan ® and Rhopress ®) after
being stored at 40° C. for 14 days

| | |
|---|---|
| Mikelan ® | Rhopress ® |

| | | |
|---|---|---|
| No. | Component | Content % |

| | | | |
|---|---|---|---|
| 1. | Netarsudil dimesylate | — | 99.3 |
| 2. | Carteolol hydrochloride | 99.5 | — |

According to Embodiments 1 to 5, the preparation including netarsudil dimesylate and carteolol mesylate has good stability after being stored under a storage condition of 40° C. for 14 days, which meets a requirement for departing from a storage condition of the commercially available eye drops (Mikelan® and Rhopress®) for a short time.

### Embodiments 6 to 10: A Preparation Including Netarsudil Dihydrochloride and Carteolol Hydrochloride

TABLE 8
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 6 | | 7 | | 8 | | 9 | | 10 | |
| 1. | Netarsudil | 11.6 | mg | 11.6 | mg | 23.3 | mg | 23.3 | mg | 46.6 | mg |
| | dihydrochloride | | | | | | | | | | |
| 2. | Carteolol | 1000 | mg | 2000 | mg | 1000 | mg | 2000 | mg | 4000 | mg |
| | hydrochloride | | | | | | | | | | |
| 3. | Boric acid | 50 | mg | 50 | mg | 50 | mg | 50 | mg | 50 | mg |
| 4. | Mannitol | 3700 | mg | 3700 | mg | 3700 | mg | 3700 | mg | 3700 | mg |
| 5. | Benzalkonium chloride | 5 | mg | 5 | mg | 5 | mg | 5 | mg | 5 | mg |
| 6. | Sodium hydroxide | q.s. | | q.s. | | q.s. | | q.s. | | q.s. | |
| 7. | Water for injection | 100 | mL | 100 | mL | 100 | mL | 100 | mL | 100 | mL |
| | added by | | | | | | | | | | |

A preparation process may refer to the preparation process in Embodiments 1 to 5.

TABLE 9
Contents of various main medicine components in Embodiments
6 to 10 after being stored at 5° C. for 24 months

| | | Embodiment | Embodiment | Embodiment | Embodiment | Embodiment |
|---|---|---|---|---|---|---|
| | | 6 | 7 | 8 | 9 | 10 |
| No. | Component | Content % | | | | |
| 1. | Netarsudil | 100.2 | 100.1 | 99.5 | 99.3 | 99.1 |
| | dihydrochloride | | | | | |
| 2. | Carteolol | 101.3 | 100.8 | 100.3 | 102.3 | 100.2 |
| | hydrochloride | | | | | |

According to Embodiments 6 to 10, the preparation including netarsudil dihydrochloride and carteolol hydrochloride has good stability after being stored under a storage condition of 5° C. for 24 months, which meets a requirement for a storage condition of the commercially available eye drops (Mikelan® and Rhopress®) before opening.

TABLE 10
Contents of various main medicine components in Embodiments
6 to 10 after being stored at 25° C. for 6 weeks

| | | Embodiment | Embodiment | Embodiment | Embodiment | Embodiment |
|---|---|---|---|---|---|---|
| | | 6 | 7 | 8 | 9 | 10 |
| No. | Component | Content % | | | | |
| 1. | Netarsudil | 97.9 | 98.4 | 99.9 | 99.1 | 99.0 |
| | dihydrochloride | | | | | |
| 2. | Carteolol | 99.7 | 99.4 | 99.5 | 99.6 | 99.8 |
| | hydrochloride | | | | | |

According to Embodiments 6 to 10, the preparation including netarsudil dihydrochloride and carteolol hydrochloride has good stability after being stored under a storage condition of 25° C. for 6 weeks, which meets a requirement for a storage condition of the commercially available eye drops (Mikelan® and Rhopress®) after opening.

TABLE 11
Contents of various main medicine components in Embodiments
6 to 10 after being stored at 40° C. for 14 days

| | | Embodiment | Embodiment | Embodiment | Embodiment | Embodiment |
|---|---|---|---|---|---|---|
| | | 6 | 7 | 8 | 9 | 10 |
| No. | Component | Content % | | | | |
| 1. | Netarsudil | 99.9 | 99.7 | 99.8 | 99.9 | 99.8 |
| | dihydrochloride | | | | | |
| 2. | Carteolol | 100.3 | 99.9 | 98.6 | 99.5 | 99.5 |
| | hydrochloride | | | | | |

According to Embodiments 6 to 10, the preparation including netarsudil dihydrochloride and carteolol hydrochloride has good stability after being stored under a storage condition of 40° C. for 14 days, which meets a requirement for departing from a storage condition of the commercially available eye drops (Mikelan® and Rhopress®) for a short time.

### Embodiment 11: Evaluation of Pharmaceutical Effects of the Ophthalmic Medicine Composition Solution in Embodiments 1 to 10 by Using a a Japanese White Rabbit Glaucoma Model

Experimental animal: healthy male Japanese white rabbit (Shenyang Pharmaceutical University, License Number: SYXK (Liaoning) 2018-0009), original weight 2.5 kg to 3.0 kg, 3 rabbits/group.

### Experimental Grouping

Normal control group: no processing.
Model group: an intraocular hypertension model is established, and no eye drop is administered.
Treatment group 1: an intraocular hypertension model is established, and commercially available netarsudil eye drops (Rhopress®) are administered.
Treatment group 2: an intraocular hypertension model is established, and commercially available carteolol hydrochloride eye drops (Mikelan®) are administered.
Embodiment group 1: an intraocular hypertension model is established, and an ophthalmic medicine composition solution in Embodiment 1 is administered.
Embodiment group 2: an intraocular hypertension model is established, and an ophthalmic medicine composition solution in Embodiment 2 is administered.
Embodiment group 3: an intraocular hypertension model is established, and an ophthalmic medicine composition solution in Embodiment 3 is administered.
Embodiment group 4: an intraocular hypertension model is established, and an ophthalmic medicine composition solution in Embodiment 4 is administered.
Embodiment group 5: an intraocular hypertension model is established, and an ophthalmic medicine composition solution in Embodiment 5 is administered.
Embodiment group 6: an intraocular hypertension model is established, and an ophthalmic medicine composition solution in Embodiment 6 is administered.
Embodiment group 7: an intraocular hypertension model is established, and an ophthalmic medicine composition solution in Embodiment 7 is administered.
Embodiment group 8: an intraocular hypertension model is established, and an ophthalmic medicine composition solution in Embodiment 8 is administered.
Embodiment group 9: an intraocular hypertension model is established, and an ophthalmic medicine composition solution in Embodiment 9 is administered.
Embodiment group 10: an intraocular hypertension model is established, and an ophthalmic medicine composition solution in Embodiment 10 is administered.

Model establishment: no processing is made to rabbits in the normal control group, urethane 0.8-1 g/kg is injected into ear veins of the other 13 groups of white rabbits on the zero day for general anesthesia, 10 μL of aqueous humor is extracted from an anterior chamber of each eye by using a 1 mL injection syringe, 10 μL compound carbomer solution (including, percentage by weight, 0.3% carbomer as a main drug and 0.025% dexamethasone) is injected, an intra-ocular pressure is measured every two days, and clinical manifestation is observed. A successful model has turbidity in the anterior chamber and significant increase of the intra-ocular pressure. An ocular hypertension rabbit model is established successfully after 10 days.

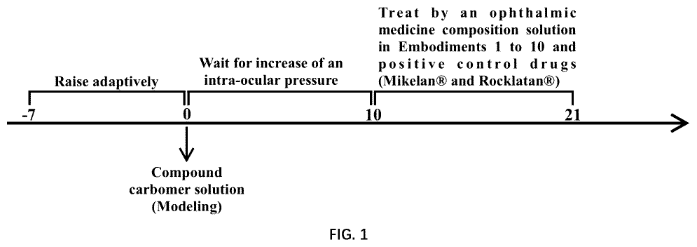

A dosage regimen: according to a current clinical dosage regimen of commercially available netarsudil eye drops (Rhopress®) and commercially available carteolol hydrochloride eye drops (Mikelan®), this experiment is mainly divided into the following groups: (1) the treatment group 1: 50 μL of the commercially available netarsudil eye drops (Rhopress®) is administered to each eye each time once a day (9:00 PM); (2) the treatment group 2: 50 μL of the commercially available carteolol hydrochloride eye drops (Mikelan®) is administered to each eye each time twice a day (9:00 AM and 9:00 PM); and (3) Embodiment groups 1 to 10: 50 μL eye drops in Embodiments 1 to 10 are administered to each eye each time once a day (9:00 PM). Drug administration is started in all the groups on the tenth day when the models are established successfully and lasts for 10 days. Eye drops are not administered to the normal control group and the model group. The dosage regimen is shown in FIG. 1.

### a Test Index and Method:

intra-ocular pressure: the intra-ocular pressure is tested once a day (9:00 PM) on the zeroth, second, fourth, sixth, eighth, tenth, eleventh, twelfth, thirteenth, fourteenth, fifteenth, sixteenth, seventeenth, eighteenth, nineteenth and twentieth days of the experiment. Upper and lower eyelids of the rabbits are opened gently, an ophthalmotonometer approaches to eyes of the rabbits, a distance is adjusted well, a measurement button is pressed gently, the eyelids are not touched during a measurement process, 6 times of measurement are completed in sequence, and then a single measurement value and an average value (mmHg) may be obtained.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 1, the treatment group 2 and Embodiment groups 1, 2, 3, 6, 7 and 8, the IOP can be reduced significantly by the ophthalmic composition solutions of Embodiment groups 4, 5, 9 and 10, however, IOP actual measured values and IOP variation values in Embodiment groups 4, 5, 9 and 10 have no significant difference.

TABLE 12
Difference values between intra-ocular pressure actual measured values of each group of
rabbits measured on the tenth day, the fifteenth day and the twentieth day and an initial
intra-ocular pressure (the intra-ocular pressure actual measured value on the tenth day)
when drug administration is performed for 10 days on end starting with the tenth day

| | IOP variation value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 1 | group 2 | group 1 | group 2 | group 3 | group 4 |
| 10 d | — | — | — | — | — | — | — | — |
| 15 d | 0.50 | −1.88 | −3.09 | −2.95 | −3.50 | −3.98 | −4.69 | −6.60 |
| 20 d | −0.50 | 2.10 | −4.43 | −4.95 | −7.90 | −8.34 | −9.07 | −12.50 |

| | IOP variation value (mmHg) | | | | | |
|---|---|---|---|---|---|---|
| Time | Embodiment | Embodiment | Embodiment | Embodiment | Embodiment | Embodiment |
| (d) | group 5 | group 6 | group 7 | group 8 | group 9 | group 10 |
| 10 d | — | — | — | — | — | — |
| 15 d | −6.86 | −4.96 | −3.82 | −3.30 | −5.80 | −5.00 |
| 20 d | −12.80 | −9.10 | −8.75 | −8.44 | −12.30 | −11.73 |

Table 13. Intra-ocular pressure actual measured values of each group of rabbits measured on the tenth day, the fifteenth day and the twentieth day when drug administration is performed for 10 days on end starting with the tenth day

TABLE 13
Intra-ocular pressure actual measured values of each group of rabbits measured
on the tenth day, the fifteenth day and the twentieth day when drug administration
is performed for 10 days on end starting with the tenth day

| | IOP actual measured value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 1 | group 2 | group 1 | group 2 | group 3 | group 4 |
| 10 d | 6.00 | 26.23 | 25.37 | 24.95 | 25.00 | 25.34 | 25.67 | 25.50 |
| 15 d | 6.50 | 24.35 | 22.28 | 22.00 | 21.50 | 21.36 | 20.98 | 18.90 |
| 20 d | 5.50 | 28.33 | 20.94 | 20.00 | 17.10 | 17.00 | 16.60 | 13.00 |

| | IOP actual measured value (mmHg) | | | | | |
|---|---|---|---|---|---|---|
| Time | Embodiment | Embodiment | Embodiment | Embodiment | Embodiment | Embodiment |
| (d) | group 5 | group 6 | group 7 | group 8 | group 9 | group 10 |
| 10 d | 26.00 | 26.50 | 26.00 | 25.30 | 25.80 | 25.00 |
| 15 d | 19.14 | 21.54 | 22.18 | 22.00 | 20.00 | 20.00 |
| 20 d | 13.20 | 17.40 | 17.25 | 16.86 | 13.50 | 13.27 |

### Embodiments 12: A Preparation Including Netarsudil Dimesylate and Carteolol Hydrobromide

TABLE 14
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol hydrobromide | 2550 | mg |
| 3. | Boric acid | 50 | mg |
| 4. | Mannitol | 4300 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 15
Contents of various main medicine components in
Embodiment 12 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.3 | 100.5 |
| 2. | Carteolol hydrobromide | 101.4 | 100.7 |

According to Embodiment 12, the preparation including netarsudil dimesylate and carteolol hydrobromide has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiments 13: A Preparation Including Netarsudil Dimesylate and Carteolol Sulfate

TABLE 16
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol sulfate | 2680 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 17
Contents of various main medicine components in
Embodiment 13 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.3 | 98.9 |
| 2. | Carteolol sulfate | 100.3 | 100.5 |

According to Embodiment 13, the preparation including netarsudil dimesylate and carteolol sulfate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 14: A Preparation Including Netarsudil Dimesylate and Carteolol Esilate

TABLE 18
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol esilate | 3340 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 19
Contents of various main medicine components in
Embodiment 14 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.5 | 101.1 |
| 2. | Carteolol esilate | 100.8 | 100.4 |

According to Embodiment 14, the preparation including netarsudil dimesylate and carteolol esilate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiments 15: A Preparation Including Netarsudil Dimesylate and Carteolol Nitrate

TABLE 20
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol nitrate | 2430 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 21
Contents of various main medicine components in
Embodiment 15 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.7 | 100.2 |
| 2. | Carteolol nitrate | 99.9 | 99.4 |

According to Embodiment 15, the preparation including netarsudil dimesylate and carteolol nitrate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiments 16: A Preparation Including Netarsudil Dimesylate and Carteolol Citrate

TABLE 22
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol citrate | 3770 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 23
Contents of various main medicine components in
Embodiment 16 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 98.5 | 99.2 |
| 2. | Carteolol citrate | 100.5 | 101.4 |

According to Embodiment 16, the preparation including netarsudil dimesylate and carteolol citrate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 17: A Preparation Including Netarsudil Dimesylate and Carteolol Tartrate

TABLE 24
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol tartrate | 3030 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 25
Contents of various main medicine components in
Embodiment 17 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.4 | 99.7 |
| 2. | Carteolol tartrate | 101.4 | 100.3 |

According to Embodiment, the preparation including netarsudil dimesylate and carteolol tartrate as goo stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiments 18: A Preparation Including Netarsudil Dimesylate and Carteolol Salicylate

TABLE 26
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol salicylate | 2940 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 27
Contents of various main medicine components in
Embodiment 18 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.9 | 100.5 |
| 2. | Carteolol salicylate | 99.3 | 99.1 |

According to Embodiment 18, the preparation including netarsudil dimesylate and carteolol salicylate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 19: A Preparation Including Netarsudil Dimesylate and Carteolol Malate

TABLE 28
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol malate | 3690 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 29
Contents of various main medicine components in
Embodiment 19 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.8 | 99.7 |
| 2. | Carteolol malate | 100.4 | 99.6 |

According to Embodiment 19, the preparation including netarsudil dimesylate and carteolol malate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 20: A Preparation Including Netarsudil Dimesylate and Carteolol Lactate

TABLE 30
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol lactate | 2620 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | ml |

A preparation process may refer to the preparation process in Embodiments 1 to 5.

TABLE 31
Contents of various main medicine components in
Embodiment 20 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.4 | 100.1 |
| 2. | Carteolol lactate | 101.2 | 100.7 |

According to Embodiment 20, the preparation including netarsudil dimesylate and carteolol lactate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 21: A Preparation Including Netarsudil Dimesylate and Carteolol Phenylacetate

TABLE 32
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Carteolol phenylacetate | 3410 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 33
Contents of various main medicine components in
Embodiment 21 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.4 | 100.1 |
| 2. | Carteolol phenylacetate | 99.9 | 99.9 |

According to Embodiment 21, the preparation including netarsudil dimesylate and carteolol phenylacetate has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 22: A Preparation Including Netarsudil Dihydrobromide and Carteolol Hydrochloride

TABLE 34
Preparation of a local ophthalmic medicine composition solution
for reducing an intra-ocular pressure as follows:

| No | Component | Weight | |
|---|---|---|---|
| 1. | Netarsudil dihydrobromide | 27.1 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 35
Contents of various main medicine components in
Embodiment 22 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dihydrobromide | 99.2 | 100.1 |
| 2. | Carteolol hydrochloride | 100.4 | 99.9 |

According to Embodiment 22, the preparation including netarsudil dihydrobromide and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 23: A Preparation Including Netarsudil Sulfate and Carteolol Hydrochloride

TABLE 36
Preparation of a local ophthalmic medicine composition solution
for reducing an intra-ocular pressure as follows:

| No. | Component | Weight | |
|---|---|---|---|
| 1. | Netarsudil sulfate | 24.3 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 37
Contents of various main medicine components in
Embodiment 23 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil sulfate | 100.2 | 100.1 |
| 2. | Carteolol hydrochloride | 99.7 | 100.1 |

According to Embodiment 23, the preparation including netarsudil sulfate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 24: A Preparation Including Netarsudil Diformate and Carteolol Hydrochloride

TABLE 38
Preparation of a local ophthalmic medicine composition solution
for reducing an intra-ocular pressure as follows:

| No. | Component | Weight | |
|---|---|---|---|
| 1. | Netarsudil diformate | 24.1 | mg |
| 2. | Carteolol hydrochloride | 2000 | mg |
| 3. | Boric acid | 50 | mg |
| 4. | Mannitol | q.s. | |
| 5. | Benzalkonium chloride | 5 | mg |
| 6. | Sodium hydroxide | q.s. | |
| 7. | Water for injection added by | 100 | mL |

TABLE 39
Contents of various main medicine components in
Embodiment 24 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diformate | 99.5 | 99.4 |
| 2. | Carteolol hydrochloride | 100.2 | 100.5 |

According to Embodiment 24, the preparation including netarsudil diformate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 25: A Preparation Including Netarsudil Nitrate and Carteolol Hydrochloride

TABLE 40
Preparation of a local ophthalmic medicine composition solution
for reducing an intra-ocular pressure as follows:

| No. | Component | Weight | |
|---|---|---|---|
| 1. | Netarsudil nitrate | 25.6 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 41
Contents of various main medicine components in
Embodiment 25 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil nitrate | 99.7 | 99.7 |
| 2. | Carteolol hydrochloride | 100.2 | 101.7 |

According to Embodiment 25, the preparation including netarsudil nitrate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 26: A Preparation Including Netarsudil Diacetate and Carteolol Hydrochloride

TABLE 42
Preparation of a local ophthalmic medicine composition solution
for reducing an intra-ocular pressure as follows:

| No. | Component | Weight | |
|---|---|---|---|
| 1. | Netarsudil diacetate | 27.8 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 43
Contents of various main medicine components in
Embodiment 26 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diacetate | 99.1 | 98.5 |
| 2. | Carteolol hydrochloride | 99.8 | 99.5 |

According to Embodiment 26, the preparation including netarsudil diacetate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 27: A Preparation Including Netarsudil Dibenzoate and Carteolol Hydrochloride

TABLE 44
Preparation of a local ophthalmic medicine composition solution
for reducing an intra-ocular pressure as follows:

| No. | Component | Weight | |
|---|---|---|---|
| 1. | Netarsudil dibenzoate | 30.8 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |

| | | |
|---|---|---|
| 6. | Sodium hydroxide | q.s. |
| 7. | Pure water | q.s. |

A preparation process may refer to the preparation process in Embodiments 1 to 5.

TABLE 45
Contents of various main medicine components in
Embodiment 27 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dibenzoate | 100.2 | 101.2 |
| 2. | Carteolol hydrochloride | 100.4 | 100.7 |

According to Embodiment 27, the preparation including netarsudil dibenzoate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 28: A Preparation Including Netarsudil Oxalate and Carteolol Hydrochloride

TABLE 46
Preparation of a local ophthalmic medicine composition solution
for reducing an intra-ocular pressure as follows:

| No. | Component | Weight | |
|---|---|---|---|
| 1. | Netarsudil oxalate | 27.9 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 47
Contents of various main medicine components in
Embodiment 28 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil oxalate | 99.9 | 100.3 |
| 2. | Carteolol hydrochloride | 100.9 | 99.8 |

According to Embodiment 28, the preparation including netarsudil oxalate and carteolol hydrochloride as goo stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 29: A Preparation Including Netarsudil Succinate and Carteolol Hydrochloride

TABLE 48
Preparation of a local ophthalmic medicine composition solution
for reducing an intra-ocular pressure as follows:

| No. | Component | Weight | |
|---|---|---|---|
| 1. | Netarsudil succinate | 27.9 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 49
Contents of various main medicine components in
Embodiment 29 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil succinate | 98.1 | 99.0 |
| 2. | Carteolol hydrochloride | 100.1 | 100.7 |

According to Embodiment 29, the preparation including netarsudil succinate and carteolol hydrochloride as good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 30: A Preparation Including Netarsudil Diphenylacetate and Carteolol Hydrochloride

TABLE 50
Preparation of a local ophthalmic medicine composition solution
for reducing an intra-ocular pressure as follows:

| No. | Component | Weight | |
|---|---|---|---|
| 1. | Netarsudil diphenylacetate | 32 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 51
Contents of various main medicine components in
Embodiment 30 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diphenylacetate | 99.4 | 99.8 |
| 2. | Carteolol hydrochloride | 99.1 | 99.0 |

According to Embodiment 30, the preparation including netarsudil diphenylacetate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Embodiment 31: A Preparation Including Netarsudil Maleate and Carteolol Hydrochloride

TABLE 52
Preparation of a local ophthalmic medicine composition solution
for reducing an intra-ocular pressure as follows:

| No. | Component | Weight | |
|---|---|---|---|
| 1. | Netarsudil maleate | 25.1 | mg |
| 2. | Carteolol hydrochloride | 2000 | mg |
| 3. | Boric acid | 50 | mg |
| 4. | Mannitol | 3700 | mg |
| 5. | Benzalkonium chloride | 5 | mg |
| 6. | Sodium hydroxide | q.s. | |
| 7. | Water for injection added by | 100 | mL |

A preparation process may refer to the preparation process in Embodiments 1 to 5.

TABLE 53
Contents of various main medicine components in
Embodiment 31 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil maleate | 99.4 | 99.8 |
| 2. | Carteolol hydrochloride | 99.1 | 99.0 |

According to Embodiment 31, the preparation including netarsudil maleate and carteolol hydrochloride has good stability after being stored under a storage condition of 5° C. for 24 months, which meets a requirement for a storage condition of the commercially available eye drops (Mikelan® and Rhopress®) before opening; and has good stability after being stored under a storage condition of 25° C. for 6 weeks, which meets a requirement for a storage condition of the commercially available eye drops (Mikelan® and Rhopress®) after opening.

### Comparative Examples 1 to 4: A Preparation Including Netarsudil Dimesylate and Carteolol Hydrochloride

TABLE 54
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 1 | | example 2 | | example 3 | | example 4 | |
| 1. | Netarsudil dimesylate | 14.25 | mg | 14.25 | mg | 28.5 | mg | 28.5 | mg |
| 2. | Carteolol hydrochloride | 1000 | mg | 2000 | mg | 1000 | mg | 2000 | mg |
| 3. | Boric acid | 50 | mg | 50 | mg | 50 | mg | 50 | mg |
| 4. | Mannitol | 3700 | mg | 3700 | mg | 3700 | mg | 3700 | mg |
| 5. | Benzalkonium chloride | 5 | mg | 5 | mg | 5 | mg | 5 | mg |
| 6. | Sodium hydroxide | q.s. | | q.s. | | q.s. | | q.s. | |
| 7. | Water for injection | 100 | mL | 100 | mL | 100 | mL | 100 | mL |
| | added by | | | | | | | | |

A preparation process may refer to the preparation process in Embodiments 1 to 5.

TABLE 55
Contents of various main medicine components in Comparative
examples 1 to 4 after being stored at 5° C. for 7 days

| | | Comparative | Comparative | Comparative | Comparative |
|---|---|---|---|---|---|
| | | example 1 | example 2 | example 3 | example 4 |
| No. | Component | Content % | | | |
| 1. | Netarsudil dimesylate | <30 | <30 | <30 | <30 |
| 2. | Carteolol hydrochloride | 101.2 | 100.8 | 100.3 | 100.2 |

According to Comparative examples 1 to 4: the preparation including netarsudil dimesylate and carteolol hydrochloride has incompatibility under the storage condition of 5° C., and netarsudil dimesylate is precipitated.

### Comparative Examples 5 to 8: A Preparation Including Netarsudil Dimesylate and Hydrochloric Acid

TABLE 56
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 5 | | example 6 | | example 7 | | example 8 | |
| 1. | Netarsudil dimesylate | 14.25 | mg | 14.25 | mg | 28.5 | mg | 28.5 | mg |
| 2. | Hydrochloric acid | 0.094 | mL | 0.188 | mL | 0.094 | mL | 0.188 | mL |
| 3. | Boric acid | 50 | mg | 50 | mg | 50 | mg | 50 | mg |
| 4. | Mannitol | 3700 | mg | 3700 | mg | 3700 | mg | 3700 | mg |
| 5. | Benzalkonium chloride | 5 | mg | 5 | mg | 5 | mg | 5 | mg |
| 6. | Sodium hydroxide | q.s. | | q.s. | | q.s. | | q.s. | |
| 7. | Water for injection | 100 | mL | 100 | mL | 100 | mL | 100 | mL |

added by

A preparation process may refer to the preparation process in Embodiments 1 to 5.

TABLE 57
Contents of various main medicine components in Comparative
examples 5 to 8 after being stored at 5° C. for 24 h

| | | Comparative | Comparative | Comparative | Comparative |
|---|---|---|---|---|---|
| | | example 5 | example 6 | example 7 | example 8 |
| No. | Component | Content % | | | |
| 1. | Netarsudil dimesylate | <30 | <30 | <30 | <30 |

According to Comparative examples 5 to 8: the preparation including netarsudil dimesylate and hydrochloric acid has incompatibility under the storage condition of 5° C., and netarsudil dimesylate is precipitated.

### Embodiments 32 to 36: A Preparation Including Netarsudil Dimesylate and Timolol Mesylate

TABLE 58
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 32 | | 33 | | 34 | | 35 | | 36 | |
| 1. | Netarsudil dimesylate | 14.25 | mg | 14.25 | mg | 28.5 | mg | 28.5 | mg | 0.057 | g |
| 2. | Timolol mesylate | 325 | mg | 650 | mg | 325 | mg | 650 | mg | 1300 | mg |
| 3. | Boric acid | 50 | mg | 50 | mg | 50 | mg | 50 | mg | 50 | mg |
| 4. | Mannitol | 4300 | mg | 4300 | mg | 4300 | mg | 4300 | mg | 4300 | mg |
| 5. | Benzalkonium chloride | 12 | mg | 12 | mg | 12 | mg | 12 | mg | 12 | mg |
| 6. | Sodium hydroxide | q.s. | | q.s. | | q.s. | | q.s. | | q.s. | |
| 7. | Water for injection | 100 | mL | 100 | mL | 100 | mL | 100 | mL | 100 | mL |
| | added by | | | | | | | | | | |

A preparation process may refer to the preparation process in Embodiments 1 to 5.

TABLE 59
Contents of various main medicine components in Embodiments
32 to 36 after being stored at 5° C. for 24 months

| | | Embodiment | Embodiment | Embodiment | Embodiment | Embodiment |
|---|---|---|---|---|---|---|
| | | 32 | 33 | 34 | 35 | 36 |
| No. | Component | Content % | | | | |
| 1. | Netarsudil | 99.8 | 100.7 | 100.3 | 100.2 | 99.8 |
| | dimesylate | | | | | |
| 2. | Timolol | 100.3 | 99.2 | 99.4 | 99.3 | 99.9 |
| | mesylate | | | | | |

TABLE 60
Contents of various main medicine components in commercially
available eye drops (Timoptic ® and Rhopress ®) after
being stored at 5° C. for 24 months

| | |
|---|---|
| Timoptic ® | Rhopress ® |

| | | |
|---|---|---|
| No. | Component | Content % |

| | | | |
|---|---|---|---|
| 1. | Netarsudil dimesylate | — | 99.7 |
| 2. | Timolol maleate | 99.8 | — |

According to Embodiments 32 to 36, the preparation including netarsudil dimesylate and timolol mesylate has good stability after being stored under a storage condition of 5° C. for 24 months, which meets a requirement for a storage condition of the commercially available eye drops (Timoptic® and Rhopress®) before opening.

TABLE 61
Contents of various main medicine components in Embodiments
32 to 36 after being stored at 25° C. for 6 weeks

| | | Embodiment | Embodiment | Embodiment | Embodiment | Embodiment |
|---|---|---|---|---|---|---|
| | | 32 | 33 | 34 | 35 | 36 |
| No. | Component | Content % | | | | |
| 1. | Netarsudil | 100.8 | 99.9 | 100.4 | 100.1 | 99.2 |
| | dimesylate | | | | | |
| 2. | Timolol | 100.2 | 100.4 | 100.2 | 99.9 | 100.4 |
| | mesylate | | | | | |

TABLE 62
Contents of various main medicine components in commercially
available eye drops (Timoptic ® and Rhopress ®) after
being stored at 25° C. for 6 weeks

| | |
|---|---|
| Timoptic ® | Rhopress ® |

| | | |
|---|---|---|
| No. | Component | Content % |

| | | | |
|---|---|---|---|
| 1. | Netarsudil dimesylate | — | 99.7 |
| 2. | Timolol maleate | 99.8 | — |

According to Embodiments 32 to 36, the preparation including netarsudil dimesylate and timolol mesylate has good stability after being stored under a storage condition of 25° C. for 6 weeks, which meets a requirement for a storage condition of the commercially available eye drops (Timoptic® and Rhopress®) after opening.

TABLE 63
Contents of various main medicine components in Embodiments
32 to 36 after being stored at 40° C. for 14 days

| | | Embodiment | Embodiment | Embodiment | Embodiment | Embodiment |
|---|---|---|---|---|---|---|
| | | 32 | 33 | 34 | 35 | 36 |
| No. | Component | Content % | | | | |
| 1. | Netarsudil | 99.2 | 100.4 | 100.6 | 100.6 | 100.2 |
| | dimesylate | | | | | |
| 2. | Timolol | 99.1 | 100.2 | 100.5 | 99.4 | 99.6 |
| | mesylate | | | | | |

TABLE 64
Contents of various main medicine components in commercially
available eye drops (Timoptic ® and Rhopress ®) after
being stored at 40° C. for 14 days

| | |
|---|---|
| Timoptic ® | Rhopress ® |

| | | |
|---|---|---|
| No. | Component | Content % |

| | | | |
|---|---|---|---|
| 1. | Netarsudil dimesylate | — | 99.7 |
| 2. | Timolol maleate | 99.8 | — |

According to Embodiments 32 to 36, the preparation including netarsudil dimesylate and timolol mesylate has good stability after being stored under a storage condition of 40° C. for 14 days, which meets a requirement for departing from a storage condition of the commercially available eye drops (Timoptic® and Rhopress®) for a short time.

### Embodiments 37 to 41: A Preparation Including Netarsudil Maleate and Timolol Maleate

TABLE 65
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 37 | | 38 | | 39 | | 40 | | 41 | |
| 1. | Netarsudil maleate | 12.6 | mg | 12.6 | mg | 25.1 | mg | 25.1 | mg | 50.2 | mg |
| 2. | Timolol maleate | 341.7 | mg | 683.5 | mg | 341.7 | mg | 683.5 | mg | 1367 | mg |
| 3. | Boric acid | 50 | mg | 50 | mg | 50 | mg | 50 | mg | 50 | mg |
| 4. | Mannitol | 4300 | mg | 4300 | mg | 4300 | mg | 4300 | mg | 4300 | mg |
| 5. | Benzalkonium | 12 | mg | 12 | mg | 12 | mg | 12 | mg | 12 | mg |
| | chloride | | | | | | | | | | |
| 6. | Sodium hydroxide | q.s. | | q.s. | | q.s. | | q.s. | | q.s. | |
| 7. | Water for injection | 100 | mL | 100 | mL | 100 | mL | 100 | mL | 100 | mL |
| | added by | | | | | | | | | | |

A preparation process may refer to the preparation process in Embodiments 1 to 5

TABLE 66
Contents of various main medicine components in Embodiments
6 to 10 after being stored at 5° C. for 24 months

| | | Embodiment | Embodiment | Embodiment | Embodiment | Embodiment |
|---|---|---|---|---|---|---|
| | | 37 | 38 | 39 | 40 | 41 |
| No. | Component | Content % | | | | |
| 1. | Netarsudil | 100.2 | 100.2 | 100.1 | 99.8 | 100.2 |
| | maleate | | | | | |
| 2. | Timolol | 100.3 | 100.3 | 99.7 | 100.2 | 100.1 |
| | maleate | | | | | |

According to Embodiments 37 to 41, the preparation including netarsudil maleate and timolol maleate has good stability after being stored under a storage condition of 5° C. for 24 months, which meets a requirement for a storage condition of the commercially available eye drops (Timoptic® and Rhopress®) before opening.

TABLE 67
Contents of various main medicine components in Embodiments
37 to 41 after being stored at 25° C. for 6 weeks

| | | Embodiment | Embodiment | Embodiment | Embodiment | Embodiment |
|---|---|---|---|---|---|---|
| | | 37 | 38 | 39 | 40 | 41 |
| No. | Component | Content % | | | | |
| 1. | Netarsudil | 99.1 | 100.2 | 100.5 | 99.6 | 100.7 |
| | maleate | | | | | |
| 2. | Timolol | 99.2 | 99.6 | 100.4 | 100.1 | 100.8 |
| | maleate | | | | | |

According to Embodiments 37 to 41, the preparation including netarsudil maleate and timolol maleate has good stability after being stored under a storage condition of 25° C. for 6 weeks, which meets a requirement for a storage condition of the commercially available eye drops (Timoptic® and Rhopress®) after opening.

TABLE 68
Contents of various main medicine components in Embodiments
37 to 41 after being stored at 40° C. for 14 days

| | | Embodiment | Embodiment | Embodiment | Embodiment | Embodiment |
|---|---|---|---|---|---|---|
| | | 37 | 38 | 39 | 40 | 41 |
| No. | Component | Content % | | | | |
| 1. | Netarsudil | 100.2 | 99.9 | 100.4 | 100.6 | 99.8 |
| | maleate | | | | | |
| 2. | Timolol | 100.1 | 99.8 | 100.2 | 99.7 | 100.2 |
| | maleate | | | | | |

According to Embodiments 37 to 41, the preparation including netarsudil maleate and timolol maleate has good stability after being stored under a storage condition of 40° C. for 14 days, which meets a requirement for departing from a storage condition of the commercially available eye drops (Timoptic® and Rhopress®) for a short time.

### Embodiment 42: Evaluation of Pharmaceutical Effects of the Ophthalmic Medicine Composition Solutions in Embodiments 32 to 41 by Using a Japanese White Rabbit Glaucoma Model

### Experimental Grouping

Normal control group: no processing.
Model group: an intraocular hypertension model is established, and no eye drop is administered.
Treatment group 1: an intraocular hypertension model is established, and commercially available netarsudil eye drops (Rhopress®) are administered.
Treatment group 3: an intraocular hypertension model is established, and commercially available timolol eye drops (Timoptic®) are administered.
Embodiment group 32: an intraocular hypertension model is established, and an ophthalmic medicine composition solution in Embodiment 32 is administered.
Embodiment group 33: an intraocular hypertension model is established, and an ophthalmic medicine composition solution in Embodiment 33 is administered.
Embodiment group 34: an intraocular hypertension model is established, and an ophthalmic medicine composition solution in Embodiment 34 is administered.
Embodiment group 35: an intraocular hypertension model is established, and an ophthalmic medicine composition solution in Embodiment 35 is administered.
Embodiment group 36: an intraocular hypertension model is established, and an ophthalmic medicine composition solution in Embodiment 36 is administered.
Embodiment group 37: an intraocular hypertension model is established, and an ophthalmic medicine composition solution in Embodiment 37 is administered.
Embodiment group 38: an intraocular hypertension model is established, and an ophthalmic medicine composition solution in Embodiment 38 is administered.
Embodiment group 39: an intraocular hypertension model is established, and an ophthalmic medicine composition solution in Embodiment 39 is administered.
Embodiment group 40: an intraocular hypertension model is established, and an ophthalmic medicine composition solution in Embodiment 40 is administered.
Embodiment group 41: an intraocular hypertension model is established, and an ophthalmic medicine composition solution in Embodiment 41 is administered.

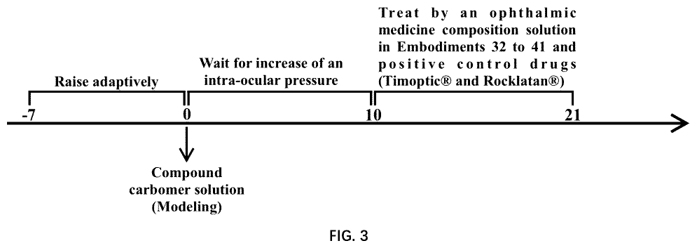

A dosage regimen: according to a current clinical dosage regimen of commercially available netarsudil eye drops (Rhopress®) and commercially available timolol eye drops (Timoptic®), this experiment is mainly divided into the following groups: (1) the treatment group 1: 50 μL the commercially available netarsudil eye drops (Rhopress®) are administered to each eye each time once a day (9:00 PM); (2) the treatment group 3: 50 μL the commercially available timolol eye drops (Timoptic®) are administered to each eye each time twice a day (9:00 AM and 9:00 PM); and (3) Embodiment groups 32 to 41: 50 μL eye drops in Embodiments 32 to 41 are administered to each eye each time once a day (9:00 PM). Drug administration is started in all the groups on the tenth day when the models are established successfully and lasts for 10 days. No processing is made to the normal control group and the model group. The dosage regimen is shown in FIG. 3.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 1, the treatment group 3 and Embodiment groups 32, 33, 34, 37, 38 and 39, the IOP can be reduced significantly by the ophthalmic composition solutions of Embodiment groups 35, 36, 40 and 41, however, IOP actual measured values and IOP variation values in Embodiment groups 35, 36, 40 and 41 have no significant difference.

TABLE 69
Difference values between intra-ocular pressure actual measured values of each group of
rabbits measured on the tenth day, the fifteenth day and the twentieth day and an initial
intra-ocular pressure (the intra-ocular pressure actual measured value on the tenth day)
when drug administration is performed for 10 days on end starting with the tenth day

| | IOP variation value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 1 | group 3 | group 32 | group 33 | group 34 | group 35 |
| 10 d | — | — | — | — | — | — | — | — |
| 15 d | 0.50 | −1.88 | −3.09 | −1.39 | −3.44 | −5.03 | −4.03 | −5.80 |
| 20 d | −0.50 | 2.10 | −6.43 | −5.98 | −9.44 | −10.50 | −10.31 | −12.82 |

| | IOP variation value (mmHg) | | | | | |
|---|---|---|---|---|---|---|
| Time | Embodiment | Embodiment | Embodiment | Embodiment | Embodiment | Embodiment |
| (d) | group 36 | group 37 | group 38 | group 39 | group 40 | group 41 |
| 10 d | — | — | — | — | — | — |
| 15 d | −8.03 | −4.70 | −5.42 | −5.34 | −5.38 | −8.00 |
| 20 d | −13.98 | −10.20 | −10.72 | −10.34 | −13.00 | −13.58 |

TABLE 70
Intra-ocular pressure actual measured values of each group of rabbits measured
on the tenth day, the fifteenth day and the twentieth day when drug administration
is performed for 10 days on end starting with the tenth day

| | IOP actual measured value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 1 | group 3 | group 32 | group 33 | group 34 | group 35 |
| 10 d | 6.00 | 26.23 | 25.37 | 25.50 | 25.77 | 27.63 | 26.63 | 26.63 |
| 15 d | 6.50 | 24.35 | 22.28 | 24.11 | 22.33 | 22.60 | 22.60 | 20.83 |
| 20 d | 5.50 | 28.33 | 18.94 | 19.52 | 16.33 | 17.13 | 16.32 | 13.81 |

| | IOP actual measured value (mmHg) | | | | | |
|---|---|---|---|---|---|---|
| Time | Embodiment | Embodiment | Embodiment | Embodiment | Embodiment | Embodiment |
| (d) | group 36 | group 37 | group 38 | group 39 | group 40 | group 41 |
| 10 d | 27.63 | 27.33 | 26.93 | 26.67 | 26.33 | 27.33 |
| 15 d | 19.60 | 22.63 | 21.51 | 21.33 | 20.95 | 19.33 |
| 20 d | 13.65 | 17.13 | 16.21 | 16.33 | 13.33 | 13.75 |

### Embodiment 43: A Preparation Including Netarsudil Dimesylate and Timolol Hydrobromide

TABLE 71
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol hydrobromide | 628 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 72
Contents of various main medicine components in
Embodiment 43 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.3 | 100.1 |
| 2. | Timolol hydrobromide | 100.2 | 100.6 |

According to Embodiment 43, the preparation including netarsudil dimesylate and timolol hydrobromide has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 44: A Preparation Including Netarsudil Dimesylate and Timolol Sulfate

TABLE 73
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol sulfate | 655 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection | 100 | mL |
| | added by | | |

A preparation process may refer to the preparation process in Embodiments 1 to 5.

TABLE 74
Contents of various main medicine components in
Embodiment 44 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.3 | 100.1 |
| 2. | Timolol sulfate | 100.2 | 99.1 |

According to Embodiment 44, the preparation including netarsudil dimesylate and timolol sulfate and having pH in a range from 4.5 to 5.4 has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 45: A Preparation Including Netarsudil Dimesylate and Timolol Esilate

TABLE 75
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol esilate | 674 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 76
Contents of various main medicine components in
Embodiment 45 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.8 | 100.1 |
| 2. | Timolol esilate | 100.2 | 100.4 |

According to Embodiment 45, the preparation including netarsudil dimesylate and timolol esilate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 46: A Preparation Including Netarsudil Dimesylate and Timolol Nitrate

TABLE 77
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol nitrate | 600 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 78
Contents of various main medicine components in
Embodiment 46 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.2 | 100.2 |
| 2. | Timolol nitrate | 100.4 | 100.8 |

According to Embodiment 46, the preparation including netarsudil dimesylate and timolol nitrate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 47: A Preparation Including Netarsudil Dimesylate and Timolol Citrate

TABLE 79
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol citrate | 804 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 80
Contents of various main medicine components in
Embodiment 47 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.2 | 100.2 |
| 2. | Timolol citrate | 100.3 | 99.3 |

According to Embodiment 47, the preparation including netarsudil dimesylate and timolol citrate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 48: A Preparation Including Netarsudil Dimesylate and Timolol Tartrate

TABLE 81
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol tartrate | 737 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 82
Contents of various main medicine components in
Embodiment 48 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.6 | 100.3 |
| 2. | Timolol tartrate | 100.3 | 99.2 |

According to Embodiment 48, the preparation including netarsudil dimesylate and timolol tartrate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 49: A Preparation Including Netarsudil Dimesylate and Timolol Salicylate

TABLE 83
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol salicylate | 718 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 84
Contents of various main medicine components in
Embodiment 49 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.5 | 100.5 |
| 2. | Timolol salicylate | 99.6 | 99.3 |

According to Embodiment 49, the preparation including netarsudil dimesylate and timolol salicylate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 50: A Preparation Including Netarsudil Dimesylate and Timolol Malate

TABLE 85
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol malate | 712 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 86
Contents of various main medicine components in
Embodiment 50 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.3 | 99.1 |
| 2. | Timolol malate | 100.6 | 100.2 |

According to Embodiment 50, the preparation including netarsudil dimesylate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 51: A Preparation Including Netarsudil Dimesylate and Timolol Lactate

TABLE 87
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol lactate | 642 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 88
Contents of various main medicine components in
Embodiment 51 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.8 | 100.9 |
| 2. | Timolol lactate | 99.6 | 100.2 |

According to Embodiment 51, the preparation including netarsudil dimesylate and timolol lactate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 52: A Preparation Including Netarsudil Dimesylate and Timolol Phenylacetate

TABLE 89
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol phenylacetate | 715 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 90
Contents of various main medicine components in
Embodiment 52 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.3 | 100.9 |
| 2. | Timolol phenylacetate | 100.2 | 100.3 |

According to Embodiment 52, the preparation including netarsudil dimesylate and timolol phenylacetate as good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 53: A Preparation Including Netarsudil Dihydrobromide and Timolol Maleate

TABLE 91
Preparation of a local ophthalmic medicine composition solution
for reducing an intra-ocular pressure as follows:

| No. | Component | Weight | |
|---|---|---|---|
| 1. | Netarsudil dihydrobromide | 27.1 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s | |
| 7. | Water for injection added by | 100 | mL |

A preparation process may refer to the preparation process in Embodiments 1 to 5.

TABLE 92
Contents of various main medicine components in
Embodiment 53 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dihydrobromide | 99.4 | 100.3 |
| 2. | Timolol maleate | 99.7 | 100.9 |

According to Embodiment 53, the preparation including netarsudil dihydrobromide and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 54: A Preparation Including Netarsudil Dihydrochloride and Timolol Maleate

TABLE 93
Preparation of a local ophthalmic medicine composition solution
for reducing an intra-ocular pressure as follows:

| No. | Component | Weight | |
|---|---|---|---|
| 1. | Netarsudil dihydrochloride | 23.3 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 94
Contents of various main medicine components in
Embodiment 54 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dihydrochloride | 99.5 | 100.3 |
| 2. | Timolol maleate | 100.5 | 99.6 |

According to Embodiment 54, the preparation including netarsudil dihydrochloride and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 55: A Preparation Including Netarsudil Sulfate and Timolol Maleate

TABLE 95
Preparation of a local ophthalmic medicine composition solution
for reducing an intra-ocular pressure as follows:

| No. | Component | Weight | |
|---|---|---|---|
| 1. | Netarsudil sulfate | 24.3 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 96
Contents of various main medicine components in
Embodiment 55 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil sulfate | 100.5 | 100.2 |
| 2. | Timolol maleate | 100.3 | 100.6 |

According to Embodiment 55, the preparation including netarsudil sulfate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 56: A Preparation Including Netarsudil Diformate and Timolol Maleate

TABLE 97
Preparation of a local ophthalmic medicine composition solution
for reducing an intra-ocular pressure as follows:

| No. | Component | Weight | |
|---|---|---|---|
| 1. | Netarsudil diformate | 24.1 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 98
Contents of various main medicine components in
Embodiment 56 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diformate | 100.3 | 100.5 |
| 2. | Timolol maleate | 99.6 | 99.7 |

According to Embodiment 56, the preparation including netarsudil diformate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 57: A Preparation Including Netarsudil Dinitrate and Timolol Maleate

TABLE 99
Preparation of a local ophthalmic medicine composition solution
for reducing an intra-ocular pressure as follows:

| No. | Component | Weight | |
|---|---|---|---|
| 1. | Netarsudil dinitrate | 25.6 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 100
Contents of various main medicine components in
Embodiment 57 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dinitrate | 99.3 | 100.8 |
| 2. | Timolol maleate | 100.2 | 100.5 |

According to Embodiment 57, the preparation including netarsudil dinitrate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 58: A Preparation Including Netarsudil Diacetate and Timolol Maleate

TABLE 101
Preparation of a local ophthalmic medicine composition solution
for reducing an intra-ocular pressure as follows:

| No. | Component | Weight | |
|---|---|---|---|
| 1. | Netarsudil diacetate | 27.8 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 102
Contents of various main medicine components in
Embodiment 58 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diacetate | 100.2 | 100.6 |
| 2. | Timolol maleate | 100.1 | 99.1 |

According to Embodiment 58, the preparation including netarsudil diacetate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 59: A Preparation Including Netarsudil Dibenzoate and Timolol Maleate

TABLE 103
Preparation of a local ophthalmic medicine composition solution
for reducing an intra-ocular pressure as follows:

| No. | Component | Weight | |
|---|---|---|---|
| 1. | Netarsudil dibenzoate | 30.8 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 104
Contents of various main medicine components in
Embodiment 59 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dibenzoate | 99.5 | 99.3 |
| 2. | Timolol maleate | 100.3 | 100.8 |

According to Embodiment 59, the preparation including netarsudil dibenzoate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 60: A Preparation Including Netarsudil Oxalate and Timolol Maleate

TABLE 105
Preparation of a local ophthalmic medicine composition solution
for reducing an intra-ocular pressure as follows:

| No. | Component | Weight | |
|---|---|---|---|
| 1. | Netarsudil oxalate | 27.9 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 106
Contents of various main medicine components in
Embodiment 60 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil oxalate | 99.4 | 100.7 |
| 2. | Timolol maleate | 99.6 | 100.8 |

According to Embodiment 60, the preparation including netarsudil oxalate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 61: A Preparation Including Netarsudil Succinate and Timolol Maleate

TABLE 107
Preparation of a local ophthalmic medicine composition solution
for reducing an intra-ocular pressure as follows:

| No. | Component | Weight | |
|---|---|---|---|
| 1. | Netarsudil succinate | 25.2 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 108
Contents of various main medicine components in
Embodiment 61 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil succinate | 100.3 | 99.2 |
| 2. | Timolol maleate | 100.6 | 100.8 |

According to Embodiment 61, the preparation including netarsudil succinate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Embodiment 62: A Preparation Including Netarsudil Diphenylacetate and Timolol Maleate

TABLE 109
Preparation of a local ophthalmic medicine composition solution
for reducing an intra-ocular pressure as follows:

| No. | Component | Weight | |
|---|---|---|---|
| 1. | Netarsudil diphenylacetate | 32 | mg |
| 2. | Timolol maleate | 683.5 | mg |
| 3. | Boric acid | 50 | mg |
| 4. | Mannitol | 4300 | mg |
| 5. | Benzalkonium chloride | 12 | mg |
| 6. | Sodium hydroxide | q.s. | |
| 7. | Water for injection added by | 100 | mL |

A preparation process may refer to the preparation process in Embodiments 1 to 5.

TABLE 110
Contents of various main medicine components in
Embodiment 62 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diphenylacetate | 99.1 | 100.6 |
| 2. | Timolol maleate | 99.3 | 100.5 |

According to Embodiment 62, the preparation including netarsudil diphenylacetate and timolol maleate has good stability after being stored under a storage condition of 5° C. for 24 months, which meets a requirement for a storage condition of the commercially available eye drops (Timoptic® and Rhopress®) before opening; and has good stability after being stored under a storage condition of 25° C. for 6 weeks, which meets a requirement for a storage condition of the commercially available eye drops (Timoptic® and Rhopress®) after opening.

### Comparative Examples 9 to 12: A Preparation Including Netarsudil Dimesylate and Timolol Maleate

TABLE 111
Preparation of a local ophthalmic medicine composition solution
for reducing an intra-ocular pressure as follows:

| | | Compar- | | Compar- | | Compar- | | Compar- | |
|---|---|---|---|---|---|---|---|---|---|
| | | ative | | ative | | ative | | ative | |
| | | exam- | | exam- | | exam- | | exam- | |
| No. | Component | ple 9 | | ple 10 | | ple 11 | | ple 12 | |
| 1. | Netarsudil | 14.25 | mg | 14.25 | mg | 28.5 | mg | 28.5 | mg |
| | dimesylate | | | | | | | | |
| 2. | Timolol | 341.7 | mg | 683.5 | mg | 341.7 | mg | 683.5 | mg |
| | maleate | | | | | | | | |
| 3. | Boric acid | 50 | mg | 50 | mg | 50 | mg | 50 | mg |
| 4. | Mannitol | 4300 | mg | 4300 | mg | 4300 | mg | 4300 | mg |
| 5. | Benzalkonium | 12 | mg | 12 | mg | 12 | mg | 12 | mg |
| | chloride | | | | | | | | |
| 6. | Sodium | q.s. | | q.s. | | q.s. | | q.s. | |
| | hydroxide | | | | | | | | |
| 7. | Water for | 100 | mL | 100 | mL | 100 | mL | 100 | mL |
| | injection | | | | | | | | |
| | added by | | | | | | | | |

A preparation process may refer to the preparation process in Embodiments 1 to 5.

TABLE 112
Contents of various main medicine components in Comparative
examples 9 to 12 after being stored at 5° C. for 7 days

| | | Compar- | Compar- | Compar- | Compar- |
|---|---|---|---|---|---|
| | | ative | ative | ative | ative |
| | | exam- | exam- | exam- | exam- |
| | | ple 9 | ple 10 | ple 11 | ple 12 |
| No. | Component | Content % | | | |
| 1. | Netarsudil | <30 | <30 | <30 | <30 |
| | dimesylate | | | | |
| 2. | Timolol | 99.3 | 99.5 | 100.3 | 100.1 |
| | maleate | | | | |

According to Comparative examples 9 to 12, the preparation including netarsudil dimesylate and timolol maleate has incompatibility under the storage condition of 5° C., and netarsudil dimesylate is precipitated.

### Comparative Examples 13 to 16: A Preparation Including Netarsudil Dimesylate and Maleic Acid

TABLE 113
Preparation of a local ophthalmic medicine composition solution
for reducing an intra-ocular pressure as follows:

| | | Compar- | | Compar- | | Compar- | | Compar- | |
|---|---|---|---|---|---|---|---|---|---|
| | | ative | | ative | | ative | | ative | |
| | | exam- | | exam- | | exam- | | exam- | |
| No. | Component | ple 13 | | ple 14 | | ple 15 | | ple 16 | |
| 1. | Netarsudil | 14.25 | mg | 14.25 | mg | 28.5 | mg | 28.5 | mg |
| | dimesylate | | | | | | | | |
| 2. | Maleic acid | 91.75 | mg | 183.5 | mg | 91.75 | mg | 183.5 | mg |
| 3. | Boric acid | 50 | mg | 50 | mg | 50 | mg | 50 | mg |
| 4. | Mannitol | 4300 | mg | 4300 | mg | 4300 | mg | 4300 | mg |
| 5. | Benzalkonium | 12 | mg | 12 | mg | 12 | mg | 12 | mg |
| | chloride | | | | | | | | |
| 6. | Sodium | q.s. | | q.s. | | q.s. | | q.s. | |
| | hydroxide | | | | | | | | |
| 7. | Water for | 100 | mL | 100 | mL | 100 | mL | 100 | mL |
| | injection | | | | | | | | |
| | added by | | | | | | | | |

A preparation process may refer to the preparation process an Embodiments 1 to 5.

TABLE 114
Contents of various main medicine components in Comparative
examples 13 to 16 after being stored at 5° C. for 7 days

| | | Compar- | Compar- | Compar- | Compar- |
|---|---|---|---|---|---|
| | | ative | ative | ative | ative |
| | | exam- | exam- | exam- | exam- |
| | | ple 13 | ple 14 | ple 15 | ple 16 |
| No. | Component | Content % | | | |
| 1. | Netarsudil | <30 | <30 | <30 | <30 |
| | dimesylate | | | | |

According to Comparative examples 13 to 16, the preparation including netarsudil dimesylate and maleic acid has incompatibility under the storage condition of 5° C., and netarsudil dimesylate is precipitated.

### Embodiments 63 to 67: A Preparation Including Netarsudil Dimesylate and Betaxolol Mesylate

TABLE 115
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 63 | | 64 | | 65 | | 66 | | 67 | |
| 1. | Netarsudil dimesylate | 14.2 | mg | 14.2 | mg | 28.5 | mg | 28.5 | mg | 57.0 | mg |
| 2. | Betaxolol mesylate | 164.1 | mg | 328.2 | mg | 164.1 | mg | 328.2 | mg | 656.3 | mg |
| 3. | Boric acid | 50 | mg | 50 | mg | 50 | mg | 50 | mg | 50 | mg |
| 4. | Mannitol | 4400 | mg | 4400 | mg | 4400 | mg | 4400 | mg | 4400 | mg |
| 5. | Benzalkonium chloride | 10 | mg | 10 | mg | 10 | mg | 10 | mg | 10 | mg |
| 6. | Sodium hydroxide | q.s. | | q.s | | q.s. | | q.s. | | q.s. | |
| 7. | Water for injection | 100 | ml | 100 | ml | 100 | ml | 100 | ml | 100 | ml |
| | added by | | | | | | | | | | |

A preparation process may refer to the preparation process in Embodiments 1 to 5.

TABLE 116
Contents of various main medicine components in Embodiments
63 to 67 after being stored at 5° C. for 24 months

| | | Embodiment | Embodiment | Embodiment | Embodiment | Embodiment |
|---|---|---|---|---|---|---|
| | | 63 | 64 | 65 | 66 | 67 |
| No. | Component | Content % | | | | |
| 1. | Netarsudil | 99.8 | 99.9 | 100.2 | 100.5 | 101.1 |
| | dimesylate | | | | | |
| 2. | Betaxolol | 99.6 | 100.2 | 99.8 | 100.1 | 100.3 |
| | mesylate | | | | | |

TABLE 117
Contents of various main medicine components in commercially
available eye drops (Betoptic ® and Rhopress ®) after
being stored at 5° C. for 24 months

| | |
|---|---|
| Betoptic ® | Rhopress ® |

| | | |
|---|---|---|
| No. | Component | Content % |

| | | | |
|---|---|---|---|
| 1. | Netarsudil | — | 100.4 |
| | dimesylate | | |
| 2. | Betaxolol | 99.5 | — |
| | hydrochloride | | |

According to Embodiments 63 to 67, the preparation including netarsudil dimesylate and betaxolol mesylate has good stability after being stored under a storage condition of 5° C. for 24 months, which meets a requirement for a storage condition of the commercially available eye drops (Betoptic® and Rhopress®) before opening.

TABLE 118
Contents of various main medicine components in Embodiments
63 to 67 after being stored at 25° C. for 6 weeks

| | | Embodiment | Embodiment | Embodiment | Embodiment | Embodiment |
|---|---|---|---|---|---|---|
| | | 63 | 64 | 65 | 66 | 67 |
| No. | Component | Content % | | | | |
| 1. | Netarsudil | 99.7 | 99.4 | 100.5 | 100.2 | 100.2 |
| | dimesylate | | | | | |
| 2. | Betaxolol | 98.5 | 99.1 | 99.6 | 99.4 | 99.9 |
| | mesylate | | | | | |

TABLE 119
Contents of various main medicine components in commercially
available eye drops (Betoptic ® and Rhopress ®) after
being stored at 25° C. for 6 weeks

| | |
|---|---|
| Betoptic ® | Rhopress ® |

| | | |
|---|---|---|
| No. | Component | Content % |

| | | | |
|---|---|---|---|
| 1. | Netarsudil dimesylate | — | 99.9 |
| 2. | Betaxolol hydrochloride | 99.9 | — |

According to Embodiments 63 to 67, the preparation including netarsudil dimesylate and betaxolol mesylate has good stability after being stored under a storage condition of 25° C. for 6 weeks, which meets a requirement for a storage condition of the commercially available eye drops (Betoptic® and Rhopress®) after opening.

TABLE 120
Contents of various main medicine components in Embodiments
63 to 67 after being stored at 40° C. for 14 days

| | | Embodiment | Embodiment | Embodiment | Embodiment | Embodiment |
|---|---|---|---|---|---|---|
| | | 63 | 64 | 65 | 66 | 67 |
| No. | Component | Content % | | | | |
| 1. | Netarsudil | 99.6 | 99.0 | 100.0 | 99.8 | 99.8 |
| | dimesylate | | | | | |
| 2. | Betaxolol | 99.8 | 100.7 | 100.4 | 99.6 | 99.4 |
| | mesylate | | | | | |

TABLE 121
Contents of various main medicine components in commercially
available eye drops (Betoptic ® and Rhopress ®) after
being stored at 40° C. for 14 days

| | |
|---|---|
| Betoptic ® | Rhopress ® |

| | | |
|---|---|---|
| No. | Component | Content % |

| | | | |
|---|---|---|---|
| 1. | Netarsudil dimesylate | — | 101.3 |
| 2. | Betaxolol hydrochloride | 100.9 | — |

According to Embodiments 63 to 67, the preparation including netarsudil dimesylate and betaxolol mesylate has good stability after being stored under a storage condition of 40° C. for 14 days, which meets a requirement for departing from a storage condition of the commercially available eye drops (Betoptic® and Rhopress®) for a short time.

### Embodiments 68 to 72: A Preparation Including Netarsudil Dihydrochloride and Betaxolol Hydrochloride

TABLE 122
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 68 | | 69 | | 70 | | 71 | | 72 | |
| 1. | Netarsudil | 11.6 | mg | 11.6 | mg | 23.3 | mg | 23.3 | mg | 46.6 | mg |
| | dihydrochloride | | | | | | | | | | |
| 2. | Betaxolol | 139.8 | mg | 279.6 | mg | 139.8 | mg | 279.6 | mg | 559.3 | mg |
| | hydrochloride | | | | | | | | | | |
| 3. | Boric acid | 50 | mg | 50 | mg | 50 | mg | 50 | mg | 50 | mg |
| 4. | Mannitol | 4400 | mg | 4400 | mg | 4400 | mg | 4400 | mg | 4400 | mg |
| 5. | Benzalkonium chloride | 10 | mg | 10 | mg | 10 | mg | 10 | mg | 10 | mg |
| 6. | Sodium hydroxide | q.s. | | q.s. | | q.s. | | q.s. | | q.s. | |
| 7. | Water for injection | 100 | mL | 100 | mL | 100 | mL | 100 | mL | 100 | mL |
| | added by | | | | | | | | | | |

A preparation process may refer to the preparation process in Embodiments 1 to 5.

TABLE 123
Contents of various main medicine components in Embodiments
68 to 72 after being stored at 5° C. for 24 months

| | | Embodiment | Embodiment | Embodiment | Embodiment | Embodiment |
|---|---|---|---|---|---|---|
| | | 68 | 69 | 70 | 71 | 72 |
| No. | Component | Content % | | | | |
| 1. | Netarsudil | 100.5 | 100.1 | 99.8 | 99.5 | 100.3 |
| | dihydrochloride | | | | | |
| 2. | Betaxolol | 98.9 | 99.5 | 99.3 | 99.9 | 100.1 |
| | hydrochloride | | | | | |

According to Embodiments 68 to 72, the preparation including netarsudil dihydrochloride and betaxolol hydrochloride has good stability after being stored under a storage condition of 5° C. for 24 months, which meets a requirement for a storage condition of the commercially available eye drops (Betoptic® and Rhopress®) before opening.

TABLE 124
Contents of various main medicine components in Embodiments
68 to 72 after being stored at 25° C. for 6 weeks

| | | Embodiment | Embodiment | Embodiment | Embodiment | Embodiment |
|---|---|---|---|---|---|---|
| | | 68 | 69 | 70 | 71 | 72 |
| No. | Component | Content % | | | | |
| 1. | Netarsudil | 99.4 | 99.7 | 100.5 | 100.2 | 99.9 |
| | dihydrochloride | | | | | |
| 2. | Betaxolol | 100.2 | 99.1 | 100.5 | 98.7 | 99.5 |
| | hydrochloride | | | | | |

According to Embodiments 68 to 72, the preparation including netarsudil dihydrochloride and betaxolol hydrochloride has good stability after being stored under a storage condition of 25° C. for 6 weeks, which meets a requirement for a storage condition of the commercially available eye drops (Betoptic® and Rhopress®) after opening.

TABLE 125
Contents of various main medicine components in Embodiments
68 to 72 after being stored at 40° C. for 14 days

| | | Embodiment | Embodiment | Embodiment | Embodiment | Embodiment |
|---|---|---|---|---|---|---|
| | | 68 | 69 | 70 | 71 | 72 |
| No. | Component | Content % | | | | |
| 1. | Netarsudil | 99.8 | 99.4 | 101.2 | 100.7 | 100.3 |
| | dihydrochloride | | | | | |
| 2. | Betaxolol | 100.5 | 100.6 | 99.8 | 100.1 | 99.9 |
| | hydrochloride | | | | | |

According to Embodiments 68 to 2, the preparation including netarsudil dihydrochloride and betaxolol hydrochloride has good stability after being stored under a storage condition of 40° C. for 14 days, which meets a requirement for departing from a storage condition of the commercially available eye drops (Betoptic® and Rhopress®) for a short time.

### Comparative Examples 17 to 20: A Preparation Including Netarsudil Dimesylate and Betaxolol Hydrochloride

TABLE 126
Preparation of a local ophthalmic medicine composition solution
for reducing an intra-ocular pressure as follows:

| | | Compar- | | Compar- | | Compar- | | Compar- | |
|---|---|---|---|---|---|---|---|---|---|
| | | ative | | ative | | ative | | ative | |
| | | exam- | | exam- | | exam- | | exam- | |
| No. | Component | ple 17 | | ple 18 | | ple 19 | | ple 20 | |
| 1. | Netarsudil | 14.25 | mg | 14.25 | mg | 28.5 | mg | 28.5 | mg |
| | dimesylate | | | | | | | | |
| 2. | Betaxolol | 139.8 | mg | 279.6 | mg | 139.8 | mg | 279.6 | mg |
| | hydrochloride | | | | | | | | |
| 3. | Boric acid | 50 | mg | 50 | mg | 50 | mg | 50 | mg |
| 4. | Mannitol | 4400 | mg | 4400 | mg | 4400 | mg | 4400 | mg |
| 5. | Benzalkonium | 10 | mg | 10 | mg | 10 | mg | 10 | mg |
| | chloride | | | | | | | | |
| 6. | Sodium | q.s. | | q.s. | | q.s. | | q.s. | |
| | hydroxide | | | | | | | | |
| 7. | Water for | 100 | mL | 100 | mL | 100 | mL | 100 | mL |
| | injection | | | | | | | | |
| | added by | | | | | | | | |

A preparation process may refer to the preparation process in Embodiments 1 to 5.

TABLE 127
Contents of various main medicine components in Comparative
examples 17 to 20 after being stored at 5° C. for 7 days

| | | Compar- | Compar- | Compar- | Compar- |
|---|---|---|---|---|---|
| | | ative | ative | ative | ative |
| | | exam- | exam- | exam- | exam- |
| | | ple 17 | ple 18 | ple 19 | ple 20 |
| No. | Component | Content % | | | |
| 1. | Netarsudil | <30 | <30 | <30 | <30 |
| | dimesylate | | | | |
| 2. | Betaxolol | 99.9 | 100.2 | 99.8 | 99.8 |
| | hydrochloride | | | | |

According to Comparative examples 17 to 20, the preparation including netarsudil dimesylate and carteolol hydrochloride has incompatibility under the storage condition of 5° C., and netarsudil dimesylate is precipitated.

### Embodiments 73 to 77: A Preparation Including Netarsudil Dimesylate and Metipranolol Mesylate

TABLE 128
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 73 | | 74 | | 75 | | 76 | | 77 | |
| 1. | Netarsudil dimesylate | 14.2 | mg | 14.2 | mg | 28.5 | mg | 28.5 | mg | 57.0 | mg |
| 2. | Metipranolol mesylate | 131.1 | mg | 393.2 | mg | 131.1 | mg | 393.2 | mg | 786.4 | mg |
| 3. | Boric acid | 50 | mg | 50 | mg | 50 | mg | 50 | mg | 50 | mg |
| 4. | Mannitol | 4600 | mg | 4600 | mg | 4600 | mg | 4600 | mg | 4600 | mg |
| 5. | Benzalkonium chloride | 10 | mg | 10 | mg | 10 | mg | 10 | mg | 10 | mg |
| 6. | Sodium hydroxide | q.s. | | q.s. | | q.s. | | q.s. | | q.s. | |
| 7. | Water for injection | 100 | ml | 100 | ml | 100 | ml | 100 | ml | 100 | ml |
| | added by | | | | | | | | | | |

A preparation process may refer to the preparation process in Embodiments 1 to 5.

TABLE 129
Contents of various main medicine components in Embodiments
73 to 77 after being stored at 5° C. for 24 months

| | | Embodiment | Embodiment | Embodiment | Embodiment | Embodiment |
|---|---|---|---|---|---|---|
| | | 73 | 74 | 75 | 76 | 77 |
| No. | Component | Content % | | | | |
| 1. | Netarsudil | 99.8 | 100.3 | 100.4 | 101.1 | 100.2 |
| | dimesylate | | | | | |
| 2. | Metipranolol | 99.8 | 99.7 | 99.5 | 99.9 | 100.0 |
| | mesylate | | | | | |

TABLE 130
Contents of various main medicine components in commercially
available eye drops (Optipranolol ® and Rhopress ®) after
being stored at 5° C. for 24 months

| | | Optipranolol ® | Rhopress ® |
|---|---|---|---|
| No. | Component | Content % | |
| 1. | Netarsudil dimesylate | — | 99.9 |
| 2. | Metipranolol hydrochloride | 99.8 | — |

According to Embodiments 73 to 77, the preparation including netarsudil dimesylate and metipranolol mesylate has good stability after being stored under a storage condition of 5° C. for 24 months, which meets a requirement for a storage condition of the commercially available eye drops (Optipranolol® and Rhopress®) before opening.

TABLE 131
Contents of various main medicine components in Embodiments
73 to 77 after being stored at 25° C. for 6 weeks

| | | Embodiment | Embodiment | Embodiment | Embodiment | Embodiment |
|---|---|---|---|---|---|---|
| | | 73 | 74 | 75 | 76 | 77 |
| No. | Component | Content % | | | | |
| 1. | Netarsudil | 100.2 | 99.8 | 100.3 | 99.6 | 99.9 |
| | dimesylate | | | | | |
| 2. | Metipranolol | 99.8 | 99.9 | 99.6 | 99.8 | 100.3 |
| | mesylate | | | | | |

TABLE 132
Contents of various main medicine components in commercially
available eye drops (Optipranolol ® and Rhopress ®) after
being stored at 25° C. for 6 weeks

| | | Optipranolol ® | Rhopress ® |
|---|---|---|---|
| No. | Component | Content % | |
| 1. | Netarsudil dimesylate | — | 99.6 |
| 2. | Metipranolol hydrochloride | 100.4 | — |

According to Embodiments 73 to 77, the preparation including netarsudil dimesylate and metipranolol mesylate has good stability after being stored under a storage condition of 25° C. for 6 weeks, which meets a requirement for a storage condition of the commercially available eye drops (Optipranolol® and Rhopress®) after opening.

TABLE 133
Contents of various main medicine components in Embodiments
73 to 77 after being stored at 40° C. for 14 days

| | | Embodiment | Embodiment | Embodiment | Embodiment | Embodiment |
|---|---|---|---|---|---|---|
| | | 73 | 74 | 75 | 76 | 77 |
| No. | Component | Content % | | | | |
| 1. | Netarsudil | 99.8 | 100.3 | 99.9 | 99.7 | 99.8 |
| | dimesylate | | | | | |
| 2. | Metipranolol | 99.9 | 100.1 | 100.7 | 99.9 | 99.7 |
| | mesylate | | | | | |

TABLE 134
Contents of various main medicine components in commercially
available eye drops (Optipranolol ® and Rhopress ®) after
being stored at 40° C. for 14 days

| | | Optipranolol ® | Rhopress ® |
|---|---|---|---|
| No. | Component | Content % | |
| 1. | Netarsudil dimesylate | — | 99.5 |
| 2. | Metipranolol hydrochloride | 99.8 | — |

According to Embodiments 73 to 77, the preparation including netarsudil dimesylate and metipranolol mesylate has good stability after being stored under a storage condition of 40° C. for 14 days, which meets a requirement for departing from a storage condition of the commercially available eye drops (Optipranolol® and Rhopress®) for a short time.

### Embodiments 78 to 82: A Preparation Including Netarsudil Dihydrochloride and Metipranolol Hydrochloride

TABLE 135
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 78 | | 79 | | 80 | | 81 | | 82 | |
| 1. | Netarsudil | 11.6 | mg | 11.6 | mg | 23.3 | mg | 23.3 | mg | 46.6 | mg |
| | dihydrochloride | | | | | | | | | | |
| 2. | Metipranolol | 111.8 | mg | 335.4 | mg | 111.8 | mg | 335.4 | mg | 670.8 | mg |
| | hydrochloride | | | | | | | | | | |
| 3. | Boric acid | 50 | mg | 50 | mg | 50 | mg | 50 | mg | 50 | mg |
| 4. | Mannitol | 4600 | mg | 4600 | mg | 4600 | mg | 4600 | mg | 4600 | mg |
| 5. | Benzalkonium | 10 | mg | 10 | mg | 10 | mg | 10 | mg | 10 | mg |
| | chloride | | | | | | | | | | |
| 6. | Sodium | q.s. | | q.s. | | q.s. | | q.s. | | q.s. | |
| | hydroxide | | | | | | | | | | |
| 7. | Water for | 100 | mL | 100 | mL | 100 | mL | 100 | mL | 100 | mL |
| | injection | | | | | | | | | | |
| | added by | | | | | | | | | | |

A preparation process may refer to the preparation process in Embodiments 1 to 5.

TABLE 136
Contents of various main medicine components in Embodiments
78 to 82 after being stored at 5° C. for 24 months

| | | Embodiment | Embodiment | Embodiment | Embodiment | Embodiment |
|---|---|---|---|---|---|---|
| | | 78 | 79 | 80 | 81 | 82 |
| No. | Component | Content % | | | | |
| 1. | Netarsudil | 100.2 | 100.1 | 99.5 | 99.3 | 99.1 |
| | dihydrochloride | | | | | |
| 2. | Metipranolol | 101.3 | 100.8 | 100.3 | 102.3 | 100.2 |
| | hydrochloride | | | | | |

According to Embodiments 78 to 82, the preparation including netarsudil dihydrochloride and metipranolol hydrochloride has good stability after being stored under a storage condition of 5° C. for 24 months, which meets a requirement for a storage condition of the commercially available eye drops (Optipranolol® and Rhopress®) before opening.

TABLE 137
Contents of various main medicine components in Embodiments
78 to 82 after being stored at 25° C. for 6 weeks

| | | Embodiment | Embodiment | Embodiment | Embodiment | Embodiment |
|---|---|---|---|---|---|---|
| | | 78 | 79 | 80 | 81 | 82 |
| No. | Component | Content % | | | | |
| 1. | Netarsudil | 97.9 | 98.4 | 99.9 | 99.1 | 99.0 |
| | dihydrochloride | | | | | |
| 2. | Metipranolol | 99.7 | 99.4 | 99.5 | 99.6 | 99.8 |
| | hydrochloride | | | | | |

According to Embodiments 78 to 82, the preparation including netarsudil dihydrochloride and metipranolol hydrochloride has good stability after being stored under a storage condition of 25° C. for 6 weeks, which meets a requirement for a storage condition of the commercially available eye drops (Optipranolol® and Rhopress®) after opening.

TABLE 138
Contents of various main medicine components in Embodiments
78 to 82 after being stored at 40° C. for 14 days

| | | Embodiment | Embodiment | Embodiment | Embodiment | Embodiment |
|---|---|---|---|---|---|---|
| | | 78 | 79 | 80 | 81 | 82 |
| No. | Component | Content % | | | | |
| 1. | Netarsudil | 99.9 | 99.7 | 99.8 | 99.9 | 99.8 |
| | dihydrochloride | | | | | |
| 2. | Metipranolol | 100.3 | 99.9 | 98.6 | 99.5 | 99.5 |
| | hydrochloride | | | | | |

According to Embodiments 78 to 82, the preparation including netarsudil dihydrochloride and metipranolol hydrochloride has good stability after being stored under a storage condition of 40° C. for 14 days, which meets a requirement for departing from a storage condition of the commercially available eye drops (Optipranolol® and Rhopress®) for a short time.

### Comparative Examples 21 to 24: A Preparation Including Netarsudil Dimesylate and Metipranolol Hydrochloride

TABLE 139
Preparation of a local ophthalmic medicine composition solution
for reducing an intra-ocular pressure as follows:

| | | Compar- | | Compar- | | Compar- | | Compar- | |
|---|---|---|---|---|---|---|---|---|---|
| | | ative | | ative | | ative | | ative | |
| | | exam- | | exam- | | exam- | | exam- | |
| No. | Component | ple 21 | | ple 22 | | ple 23 | | ple 24 | |
| 1. | Netarsudil | 14.25 | mg | 14.25 | mg | 28.5 | mg | 28.5 | mg |
| | dimesylate | | | | | | | | |
| 2. | Metipranolol | 111.8 | mg | 335.4 | mg | 111.8 | mg | 335.4 | mg |
| | hydrochloride | | | | | | | | |
| 3. | Boric acid | 50 | mg | 50 | mg | 50 | mg | 50 | mg |
| 4. | Mannitol | 4600 | mg | 4600 | mg | 4600 | mg | 4600 | mg |
| 5. | Benzalkonium | 10 | mg | 10 | mg | 10 | mg | 10 | mg |
| | chloride | | | | | | | | |
| 6. | Sodium | q.s. | | q.s. | | q.s. | | q.s. | |
| | hydroxide | | | | | | | | |
| 7. | Water for | 100 | mL | 100 | mL | 100 | mL | 100 | mL |
| | injection | | | | | | | | |
| | added by | | | | | | | | |

A preparation process may refer to the preparation process in Embodiments 1 to 5.

TABLE 140
Contents of various main medicine components in Comparative
examples 21 to 24 after being stored at 5° C. for 7 days

| | | Compar- | Compar- | Compar- | Compar- |
|---|---|---|---|---|---|
| | | ative | ative | ative | ative |
| | | exam- | exam- | exam- | exam- |
| | | ple 21 | ple 22 | ple 23 | ple 24 |
| No. | Component | Content % | | | |
| 1. | Netarsudil | <30 | <30 | <30 | <30 |
| | dimesylate | | | | |
| 2. | Metipranolol | 101.2 | 100.8 | 100.3 | 100.2 |
| | hydrochloride | | | | |

According to Comparative examples 21 to 24, the preparation including netarsudil dimesylate and metipranolol hydrochloride has incompatibility under the storage condition of 5° C., and netarsudil dimesylate is precipitated.

### Embodiments 83 to 87: A Preparation Including Netarsudil Dimesylate, Timolol Mesylate and Latanoprost

TABLE 141
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 83 | | 84 | | 85 | | 86 | | 87 | |
| 1. | Netarsudil dimesylate | 14.2 | mg | 14.2 | mg | 28.5 | mg | 28.5 | mg | 57.0 | mg |
| 2. | Timolol mesylate | 325 | mg | 650 | mg | 325 | mg | 650 | mg | 1300 | mg |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process is as follows: 1) 50% of a prescription dosage of water for injection is weighed, prescription dosages of prostaglandins active components and benzalkonium chloride are added and heating in water bath and stirring are performed to be dissolved completely; 2) 45% of the prescription dosage of water for injection is weighed, prescription dosages of other active components and adjuvants are added, and stirring is performed to be dissolved completely; 3) the solution in step 2 is added into the solution in step 1, stirring is performed to be uniform, and pH is regulated with a sodium hydroxide solution (10%) to a range from 4.5 to 5.4; 4) water for injection is supplemented to a full prescription dosage, stirring is performed to be uniform, and a volume reaches 100%; and 5) the solution in step 4 is put in a low-density polyethylene medicinal eye drop bottle, and its stability is studied under different storage temperature conditions.

TABLE 142
Contents of various main medicine components in Embodiments
83 to 87 after being stored at 5° C. for 24 months

| | | Embodiment | Embodiment | Embodiment | Embodiment | Embodiment |
|---|---|---|---|---|---|---|
| | | 83 | 84 | 85 | 86 | 87 |
| No. | Component | Content % | | | | |
| 1. | Netarsudil dimesylate | 99.9 | 99.7 | 100.2 | 100.1 | 99.6 |
| 2. | Timolol mesylate | 101.3 | 100.2 | 100.4 | 99.8 | 99.7 |
| 3. | Latanoprost | 100.2 | 99.8 | 99.6 | 99.2 | 100.5 |

TABLE 143
Contents of various main medicine components in commercially
available eye drops (Timoptic ®, Rhopress ®, and
Xalatan ®) after being stored at 5° C. for 24 months

| | | Timoptic ® | Rhopress ® | Xalatan ® |
|---|---|---|---|---|
| No. | Component | Content % | | |
| 1. | Timolol maleate | 99.7 | — | — |
| 2. | Netarsudil dimesylate | — | 99.9 | — |
| 3. | Latanoprost | — | — | 99.8 |

According to Embodiments 83 to 87, the preparation including netarsudil dimesylate, timolol mesylate and latanoprost has good stability after being stored under a storage condition of 5° C. for 24 months, which meets a requirement for a storage condition of the commercially available eye drops (Timoptic®, Rhopress®, and Xalatan®) before opening.

TABLE 144
Contents of various main medicine components in Embodiments
83 to 87 after being stored at 25° C. for 6 weeks

| | | Embodiment | Embodiment | Embodiment | Embodiment | Embodiment |
|---|---|---|---|---|---|---|
| | | 83 | 84 | 85 | 86 | 87 |
| No. | Component | Content % | | | | |
| 1. | Netarsudil dimesylate | 99.8 | 98.9 | 101.4 | 100.4 | 99.7 |
| 2. | Timolol mesylate | 100.3 | 99.4 | 100.6 | 98.9 | 100.6 |
| 3. | Latanoprost | 100.9 | 100.1 | 101.4 | 99.6 | 100.1 |

TABLE 145
Contents of various main medicine components in commercially
available eye drops (Timoptic ®, Rhopress ®, and
Xalatan ®) after being stored at 25° C. for 6 weeks

| | | Timoptic ® | Rhopress ® | Xalatan ® |
|---|---|---|---|---|
| No. | Component | Content % | | |
| 1. | Timolol maleate | 99.5 | — | — |
| 2. | Netarsudil dimesylate | — | 99.8 | — |
| 3. | Latanoprost | — | — | 99.6 |

According to Embodiments 83 to 87, the preparation including netarsudil dimesylate, timolol mesylate and latanoprost has good stability after being stored under a storage condition of 25° C. for 6 weeks, which meets a requirement for a storage condition of the commercially available eye drops (Timoptic®, Rhopress®, and Xalatan®) after opening.

TABLE 146
Contents of various main medicine components in Embodiments
83 to 87 after being stored at 40° C. for 14 days

| | | Embodiment | Embodiment | Embodiment | Embodiment | Embodiment |
|---|---|---|---|---|---|---|
| | | 83 | 84 | 85 | 86 | 87 |
| No. | Component | Content % | | | | |
| 1. | Netarsudil dimesylate | 100.2 | 101.4 | 100.8 | 99.6 | 100.3 |
| 2. | Timolol mesylate | 100.1 | 100.5 | 99.5 | 99.6 | 99.1 |
| 3. | Latanoprost | 99.1 | 99.2 | 100.1 | 100.8 | 100.1 |

TABLE 147
Contents of various main medicine components in commercially
available eye drops (Timoptic ®, Rhopress ®, and
Xalatan ®) after being stored at 40° C. for 14 days

| | | Timoptic ® | Rhopress ® | Xalatan ® |
|---|---|---|---|---|
| No. | Component | Content % | | |
| 1. | Timolol maleate | 99.8 | — | — |
| 2. | Netarsudil dimesylate | — | 99.5 | — |
| 3. | Latanoprost | — | — | 99.7 |

According to Embodiments 83 to 87, the preparation including netarsudil dimesylate, timolol mesylate and latanoprost has good stability after being stored under a storage condition of 40° C. for 14 days, which meets a requirement for departing from a storage condition of the commercially available eye drops (Timoptic®, Rhopress®, and Xalatan®) for a short time.

### Embodiments 88 to 92: A Preparation Including Netarsudil Maleate, Timolol Maleate and Latanoprost

TABLE 148
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 88 | | 89 | | 90 | | 91 | | 92 | |
| 1. | Netarsudil maleate | 12.6 | mg | 12.6 | mg | 25.1 | mg | 25.1 | mg | 50.2 | mg |
| 2. | Timolol maleate | 341.7 | mg | 683.5 | mg | 341.7 | mg | 683.5 | mg | 1367 | mg |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium | 20 | mg | 20 | mg | 20 | mg | 20 | mg | 20 | mg |
| | chloride | | | | | | | | | | |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 149
Contents of various main medicine components in Embodiments
88 to 92 after being stored at 5° C. for 24 months

| | | Embodiment | Embodiment | Embodiment | Embodiment | Embodiment |
|---|---|---|---|---|---|---|
| | | 88 | 89 | 90 | 91 | 92 |
| No. | Component | Content % | | | | |
| 1. | Netarsudil maleate | 100.1 | 100.4 | 100.5 | 99.5 | 99.2 |
| 2. | Timolol maleate | 100.8 | 99.3 | 99.4 | 100.1 | 100.3 |
| 3. | Latanoprost | 101.2 | 100.1 | 99.3 | 99.4 | 100.1 |

According to Embodiments 88 to 92, the preparation including netarsudil maleate, timolol maleate and latanoprost has good stability after being stored under a storage condition of 5° C. for 24 months, which meets a requirement for a storage condition of the commercially available eye drops (Timoptic®, Rhopress®, and Xalatan®) before opening.

TABLE 150
Contents of various main medicine components in Embodiments
88 to 92 after being stored at 25° C. for 6 weeks

| | | Embodiment | Embodiment | Embodiment | Embodiment | Embodiment |
|---|---|---|---|---|---|---|
| | | 88 | 89 | 90 | 91 | 92 |
| No. | Component | Content % | | | | |
| 1. | Netarsudil maleate | 100.1 | 100.8 | 99.5 | 99.8 | 100.5 |
| 2. | Timolol maleate | 99.3 | 99.3 | 100.5 | 100.4 | 100.1 |
| 3. | Latanoprost | 99.2 | 100.1 | 100.7 | 99.1 | 98.9 |

According to Embodiments 88 to 92, the preparation including netarsudil maleate, timolol maleate and latanoprost has good stability after being stored under a storage condition of 25° C. for 6 weeks, which meets a requirement for a storage condition of the commercially available eye drops (Timoptic®, Rhopress®, and Xalatan®) after opening.

TABLE 151
Contents of various main medicine components in Embodiments
88 to 92 after being stored at 40° C. for 14 days

| | | Embodiment | Embodiment | Embodiment | Embodiment | Embodiment |
|---|---|---|---|---|---|---|
| | | 88 | 89 | 90 | 91 | 92 |
| No. | Component | Content % | | | | |
| 1. | Netarsudil maleate | 100.1 | 98.9 | 100.9 | 100.1 | 98.8 |
| 2. | Timolol maleate | 100.9 | 100.8 | 100.5 | 99.9 | 100.1 |
| 3. | Latanoprost | 99.3 | 99.1 | 100.1 | 99.5 | 100.9 |

According to Embodiments 88 to 92, the preparation including netarsudil maleate, timolol maleate and latanoprost has good stability after being stored under a storage condition of 40° C. for 14 days, which meets a requirement for departing from a storage condition of the commercially available eye drops (Timoptic®, Rhopress®, and Xalatan®) for a short time.

### Embodiment 93: Evaluation of Pharmaceutical Effects of the Ophthalmic Medicine Composition Solution in Embodiments 83 to 92 by Using a a Japanese White Rabbit Glaucoma Model

### Experimental Grouping

Normal control group: no processing.
Model group: an intraocular hypertension model is established, and no eye drop is administered.
Treatment group 4: an intraocular hypertension model is established, and commercially available latanoprost and timolol eye drops (Xalacom®) are administered.
Treatment group 5: an intraocular hypertension model is established, and commercially available latanoprost and netarsudil eye drops (Rocklatan®) are administered.
Embodiment group 83: an intraocular hypertension model is established, and an ophthalmic medicine composition solution in Embodiment 83 is administered.
Embodiment group 84: an intraocular hypertension model is established, and an ophthalmic medicine composition solution in Embodiment 84 is administered.
Embodiment group 85: an intraocular hypertension model is established, and an ophthalmic medicine composition solution in Embodiment 85 is administered.
Embodiment group 86: an intraocular hypertension model is established, and an ophthalmic medicine composition solution in Embodiment 86 is administered.
Embodiment group 87: an intraocular hypertension model is established, and an ophthalmic medicine composition solution in Embodiment 87 is administered.
Embodiment group 88: an intraocular hypertension model is established, and an ophthalmic medicine composition solution in Embodiment 88 is administered.
Embodiment group 89: an intraocular hypertension model is established, and an ophthalmic medicine composition solution in Embodiment 89 is administered.
Embodiment group 90: an intraocular hypertension model is established, and an ophthalmic medicine composition solution in Embodiment 90 is administered.
Embodiment group 91: an intraocular hypertension model is established, and an ophthalmic medicine composition solution in Embodiment 91 is administered.
Embodiment group 92: an intraocular hypertension model is established, and an ophthalmic medicine composition solution in Embodiment 92 is administered.

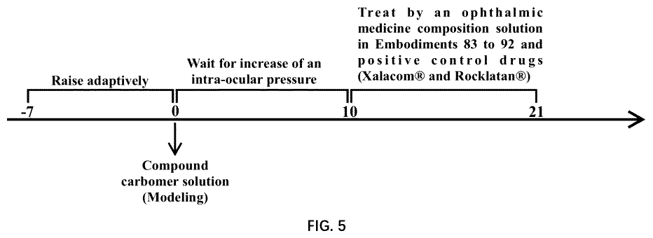

A dosage regimen: according to a current clinical dosage regimen of commercially available latanoprost and timolol eye drops (Xalacom®) and commercially available latanoprost and netarsudil eye drops (Rocklatan®), this experiment is mainly divided into the following groups: (1) the treatment group 4: 50 μL the commercially available latanoprost and timolol eye drops (Xalacom®) is administered to each eye each time once a day (9:00 PM); (2) the treatment group 5: 50 μL the commercially available latanoprost and netarsudil eye drops (Rocklatan®) is administered to each eye each time once a day (9:00 PM); and (3) Embodiment groups 83 to 92: 50 μL eye drops in Embodiments 83 to 92 are administered to each eye each time once a day (9:00 PM). Drug administration is started in all the groups on the tenth day when the models are established successfully and lasts for 10 days. No processing is made to the normal control group and the model group. The dosage regimen is shown in FIG. 5.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 4, the treatment group 5 and Embodiment groups 83, 84, 85, 88, 89 and 90, the IOP can be reduced significantly by the ophthalmic composition solutions of Embodiment groups 86, 87, 91 and 92, however, IOP actual measured values and IOP variation values in Embodiment groups 86, 87, 91 and 92 have no significant difference.

TABLE 152
Difference values between intra-ocular pressure actual measured values of each group of
rabbits measured on the tenth day, the fifteenth day and the twentieth day and an initial
intra-ocular pressure (the intra-ocular pressure actual measured value on the tenth day)
when drug administration is performed for 10 days on end starting with the tenth day

| | IOP variation value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 4 | group 5 | group 83 | group 84 | group 85 | group 86 |
| 10 d | — | — | — | — | — | — | — | — |
| 15 d | 0.50 | 2.25 | 1.84 | 1.50 | −6.00 | −6.50 | −6.00 | −3.50 |
| 20 d | −0.50 | 8.58 | −4.33 | −4.50 | −8.00 | −9.50 | −10.50 | −14.00 |

| | IOP variation value (mmHg) | | | | | |
|---|---|---|---|---|---|---|
| Time | Embodiment | Embodiment | Embodiment | Embodiment | Embodiment | Embodiment |
| (d) | group 87 | group 88 | group 89 | group 90 | group 91 | group 92 |
| 10 d | — | — | — | — | — | — |
| 15 d | −8.50 | −4.00 | −5.00 | −4.50 | −6.60 | −8.50 |
| 20 d | −14.00 | −8.00 | −10.50 | −10.50 | −14.50 | −14.00 |

TABLE 153
Intra-ocular pressure actual measured values of each group of rabbits measured on the tenth day, the fifteenth
day and the twentieth day when drug administration is performed for 10 days on end starting with the tenth day

| | IOP actual measured value (mmHg) | | | | | | | | | | | | | |
|---|---|---|---|---|---|---|---|---|---|---|---|---|---|---|
| | | | Treat- | Treat- | Embo- | Embo- | Embo- | Embo- | Embo- | Embo- | Embo- | Embo- | Embo- | Embo- |
| | Normal | | ment | ment | diment | diment | diment | diment | diment | diment | diment | diment | diment | diment |
| Time | control | Model | group | group | group | group | group | group | group | group | group | group | group | group |
| (d) | group | group | 4 | 5 | 83 | 84 | 85 | 86 | 87 | 88 | 89 | 90 | 91 | 92 |
| 10 d | 6.00 | 26.75 | 24.33 | 24.83 | 25.00 | 24.50 | 26.00 | 25.00 | 24.00 | 25.00 | 26.00 | 25.50 | 25.50 | 24.00 |
| 15 d | 6.50 | 29.00 | 26.17 | 26.33 | 19.00 | 18.00 | 20.00 | 21.50 | 15.50 | 21.00 | 21.00 | 21.00 | 18.90 | 15.50 |
| 20 d | 5.50 | 35.33 | 20.00 | 20.33 | 17.00 | 15.00 | 15.50 | 11.00 | 10.00 | 17.00 | 15.50 | 15.00 | 11.00 | 10.00 |

### Embodiments 94 to 98: A Preparation Including Netarsudil Dimesylate, Timolol Mesylate and Bimatoprost

TABLE 154
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 94 | | 95 | | 96 | | 97 | | 98 | |
| 1. | Netarsudil dimesylate | 14.2 | mg | 14.2 | mg | 28.5 | mg | 28.5 | mg | 57.0 | mg |
| 2. | Timolol mesylate | 325 | mg | 650 | mg | 325 | mg | 650 | mg | 1300 | mg |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 5 | mg | 5 | mg | 5 | mg | 5 | mg | 5 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 155
Contents of various main medicine components in Embodiments
94 to 98 after being stored at 5° C. for 24 months

| | | Embodiment | Embodiment | Embodiment | Embodiment | Embodiment |
|---|---|---|---|---|---|---|
| | | 94 | 95 | 96 | 97 | 98 |
| No. | Component | Content % | | | | |
| 1. | Netarsudil dimesylate | 99.6 | 100.7 | 100.4 | 101.1 | 100.6 |
| 2. | Timolol mesylate | 100.3 | 101.0 | 99.4 | 99.9 | 100.7 |
| 3. | Bimatoprost | 99.2 | 99.6 | 100.6 | 99.9 | 100.2 |

TABLE 156
Contents of various main medicine components in commercially
available eye drops (Timoptic ®, Rhopress ®, and
Lumigan ®) after being stored at 5° C. for 24 months

| | | Timoptic ® | Rhopress ® | Lumigan ® |
|---|---|---|---|---|
| No. | Component | Content % | | |
| 1. | Timolol maleate | 99.7 | — | — |
| 2. | Netarsudil dimesylate | — | 99.9 | — |
| 3. | Bimatoprost | — | — | 99.4 |

According to Embodiments 94 to 98, the preparation including netarsudil dimesylate, timolol mesylate and bimatoprost has good stability after being stored under a storage condition of 5° C. for 24 months, which meets a requirement for a storage condition of the commercially available eye drops (Timoptic®, Rhopress®, and Lumigan®) before opening.

TABLE 157
Contents of various main medicine components in Embodiments
94 to 98 after being stored at 25° C. for 6 weeks

| | | Embodiment | Embodiment | Embodiment | Embodiment | Embodiment |
|---|---|---|---|---|---|---|
| | | 94 | 95 | 96 | 97 | 98 |
| No. | Component | Content % | | | | |
| 1. | Netarsudil dimesylate | 99.2 | 99.9 | 101.2 | 100.1 | 99.1 |
| 2. | Timolol mesylate | 99.3 | 99.8 | 101.6 | 99.9 | 99.6 |
| 3. | Bimatoprost | 100.3 | 100.3 | 101.4 | 99.2 | 100.6 |

TABLE 158
Contents of various main medicine components in commercially
available eye drops (Timoptic ®, Rhopress ®, and
Lumigan ®) after being stored at 25° C. for 6 weeks

| | | Timoptic ® | Rhopress ® | Lumigan ® |
|---|---|---|---|---|
| No. | Component | Content % | | |
| 1. | Timolol maleate | 99.5 | — | — |
| 2. | Netarsudil dimesylate | — | 99.8 | — |
| 3. | Bimatoprost | — | — | 99.5 |

According to Embodiments 94 to 98, the preparation including netarsudil dimesylate, timolol mesylate and bimatoprost has good stability after being stored under a storage condition of 25° C. for 6 weeks, which meets a requirement for a storage condition of the commercially available eye drops (Timoptic®, Rhopress®, and Lumigan®) after opening.

TABLE 159
Contents of various main medicine components in Embodiments
94 to 98 after being stored at 40° C. for 14 days

| | | Embodiment | Embodiment | Embodiment | Embodiment | Embodiment |
|---|---|---|---|---|---|---|
| | | 94 | 95 | 96 | 97 | 98 |
| No. | Component | Content % | | | | |
| 1. | Netarsudil dimesylate | 101.2 | 101.1 | 99.8 | 99.1 | 99.3 |
| 2. | Timolol mesylate | 101.1 | 101.5 | 101.2 | 99.4 | 99.9 |
| 3. | Bimatoprost | 99.4 | 99.9 | 100.9 | 100.1 | 100.2 |

TABLE 160
Contents of various main medicine components in commercially
available eye drops (Timoptic ®, Rhopress ®, and
Lumigan ®) after being stored at 40° C. for 14 days

| | | Timoptic ® | Rhopress ® | Lumigan ® |
|---|---|---|---|---|
| No. | Component | Content % | | |
| 1. | Timolol maleate | 99.8 | — | — |
| 2. | Netarsudil dimesylate | — | 99.5 | — |
| 3. | Bimatoprost | — | — | 99.2 |

According to Embodiments 94 to 98, the preparation including netarsudil dimesylate, timolol mesylate and bimatoprost has good stability after being stored under a storage condition of 40° C. for 14 days, which meets a requirement for departing from a storage condition of the commercially available eye drops (Timoptic®, Rhopress®, and Lumigan®) for a short time.

### Embodiments 99 to 103: A Preparation Including Netarsudil Maleate, Timolol Maleate and Bimatoprost

TABLE 161
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 99 | | 100 | | 101 | | 102 | | 103 | |
| 1. | Netarsudil maleate | 12.6 | mg | 12.6 | mg | 25.1 | mg | 25.1 | mg | 50.2 | mg |
| 2. | Timolol mesylate | 341.7 | mg | 683.5 | mg | 341.7 | mg | 683.5 | mg | 1367 | mg |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 5 | mg | 5 | mg | 5 | mg | 5 | mg | 5 | mg |
| 7. | Sodium hydroxide | q.s | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 162
Contents of various main medicine components in Embodiments
99 to 103 after being stored at 5° C. for 24 months

| | | Embodiment | Embodiment | Embodiment | Embodiment | Embodiment |
|---|---|---|---|---|---|---|
| No. | Component | 99 | 100 | 101 | 102 | 103 |
| 1. | Netarsudil maleate | 99.1 | 100.4 | 100.8 | 99.3 | 98.2 |
| 2. | Timolol maleate | 99.8 | 99.4 | 99.4 | 100.2 | 102.1 |
| 3. | Bimatoprost | 101.1 | 99.1 | 99.5 | 99.3 | 100.1 |

According to Embodiments 99 to 103, the preparation including netarsudil maleate, timolol maleate and bimatoprost has good stability after being stored under a storage condition of 5° C. for 24 months, which meets a requirement for a storage condition of the commercially available eye drops (Timoptic®, Rhopress®, and Lumigan®) before opening.

TABLE 163
Contents of various main medicine components in Embodiments
99 to 103 after being stored at 25° C. for 6 weeks

| | | Embodiment | Embodiment | Embodiment | Embodiment | Embodiment |
|---|---|---|---|---|---|---|
| | | 99 | 100 | 101 | 102 | 103 |
| No. | Component | Content % | | | | |
| 1. | Netarsudil maleate | 99.1 | 100.1 | 99.2 | 99.1 | 100.6 |
| 2. | Timolol maleate | 99.9 | 98.3 | 98.5 | 98.4 | 101.1 |
| 3. | Bimatoprost | 98.5 | 100.3 | 100.4 | 101.1 | 99.1 |

According to Embodiments 99 to 103, the preparation including netarsudil maleate, timolol maleate and bimatoprost has good stability after being stored under a storage condition of 25° C. for 6 weeks, which meets a requirement for a storage condition of the commercially available eye drops (Timoptic®, Rhopress®, and Lumigan®) after opening.

TABLE 164
Contents of various main medicine components after being stored at 40° C. for 14 days

| | | Embodiment | Embodiment | Embodiment | Embodiment | Embodiment |
|---|---|---|---|---|---|---|
| | | 99 | 100 | 101 | 102 | 103 |
| No. | Component | Content % | | | | |
| 1. | Netarsudil maleate | 99.1 | 99.9 | 100.1 | 101.1 | 98.3 |
| 2. | Timolol maleate | 100.1 | 101.8 | 100.3 | 98.1 | 101.1 |
| 3. | Bimatoprost | 98.3 | 99.5 | 100.9 | 99.9 | 100.2 |

According to Embodiments 99 to 103, the preparation including netarsudil maleate, timolol maleate and bimatoprost T has good stability after being stored under a storage condition of 40° C. for 14 days, which meets a requirement for departing from a storage condition of the commercially available eye drops (Timoptic®, Rhopress®, and Lumigan®) for a short time.

### Embodiment 104: Evaluation of Pharmaceutical Effects of the Ophthalmic Medicine Composition Solutions in Embodiments 94 to 103 by Using a Japanese White Rabbit Glaucoma Model

### Experimental Grouping

Normal control group: no processing.
Model group: an intraocular hypertension model is established, and no eye drop is administered.
Treatment group 6: an intraocular hypertension model is established, and commercially available bimatoprost and timolol maleate eye drops (Ganfort®) are administered.
Embodiment group 94: an intraocular hypertension model is established, and an ophthalmic medicine composition solution in Embodiment 94 is administered.
Embodiment group 95: an intraocular hypertension model is established, and an ophthalmic medicine composition solution in Embodiment 95 is administered.
Embodiment group 96: an intraocular hypertension model is established, and an ophthalmic medicine composition solution in Embodiment 96 is administered.
Embodiment group 97: an intraocular hypertension model is established, and an ophthalmic medicine composition solution in Embodiment 97 is administered.
Embodiment group 98: an intraocular hypertension model is established, and an ophthalmic medicine composition solution in Embodiment 98 is administered.
Embodiment group 99: an intraocular hypertension model is established, and an ophthalmic medicine composition solution in Embodiment 99 is administered.
Embodiment group 100: an intraocular hypertension model is established, and an ophthalmic medicine composition solution in Embodiment 100 is administered.
Embodiment group 101: an intraocular hypertension model is established, and an ophthalmic medicine composition solution in Embodiment 101 is administered.
Embodiment group 102: an intraocular hypertension model is established, and an ophthalmic medicine composition solution in Embodiment 102 is administered.
Embodiment group 103: an intraocular hypertension model is established, and an ophthalmic medicine composition solution in Embodiment 103 is administered.

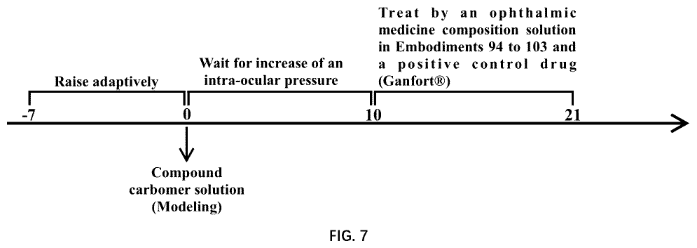

A dosage regimen: according to a current clinical dosage regimen of commercially available bimatoprost and timolol maleate eye drops (Ganfort®), this experiment is mainly divided into the following groups: (1) the treatment group 6: 50 μL the commercially available bimatoprost and timolol maleate eye drops (Ganfort®) are administered to each eye each time once a day (9:00 PM); and (2) Embodiment groups 94 to 103: 50 μL eye drops in Embodiments 94 to 103 are administered to each eye each time once a day (9:00 PM). Drug administration is started in all the groups on the tenth day when the models are established successfully and lasts for 10 days. No processing is made to the normal control group and the model group. The dosage regimen is shown in FIG. 7.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 6 and Embodiment groups 94, 95, 96, 99, 100 and 101, the IOP can be reduced significantly by the ophthalmic composition solutions of Embodiment groups 97, 98, 102 and 103, however, IOP actual measured values and IOP variation values in Embodiment groups 97, 98, 102 and 103 have no significant difference.

TABLE 165
Difference values between intra-ocular pressure actual measured values of each group of rabbits measured on the tenth
day, the fifteenth day and the twentieth day and an initial intra-ocular pressure (the intra-ocular pressure actual
measured value on the tenth day) when drug administration is performed for 10 days on end starting with the tenth day

| | IOP variation value (mmHg) | | | | | | | | | | | | |
|---|---|---|---|---|---|---|---|---|---|---|---|---|---|
| | | | Treat- | Embo- | Embo- | Embo- | Embo- | Embo- | Embo- | Embo- | Embo- | Embo- | Embo- |
| | Normal | | ment | diment | diment | diment | diment | diment | diment | diment | diment | diment | diment |
| Time | control | Model | group | group | group | group | group | group | group | group | group | group | group |
| (d) | group | group | 6 | 94 | 95 | 96 | 97 | 98 | 99 | 100 | 101 | 102 | 103 |
| 10 d | — | — | — | — | — | — | — | — | — | — | — | — | — |
| 15 d | 0.50 | 2.25 | −0.83 | −5.00 | −6.00 | −6.00 | −6.70 | −7.50 | −3.50 | −5.00 | −6.30 | −7.30 | −6.60 |
| 20 d | 0.50 | 8.58 | −4.50 | −8.70 | −9.20 | −11.00 | −15.20 | −14.00 | −8.30 | −10.70 | −9.70 | −14.30 | −14.50 |

TABLE 166
Intra-ocular pressure actual measured values of each group of rabbits measured on the tenth day, the fifteenth
day and the twentieth day when drug administration is performed for 10 days on end starting with the tenth day

| | IOP actual measured value (mmHg) | | | | | | | | | | | | |
|---|---|---|---|---|---|---|---|---|---|---|---|---|---|
| | | | Treat- | Embo- | Embo- | Embo- | Embo- | Embo- | Embo- | Embo- | Embo- | Embo- | Embo- |
| | Normal | | ment | diment | diment | diment | diment | diment | diment | diment | diment | diment | diment |
| Time | control | Model | group | group | group | group | group | group | group | group | group | group | group |
| (d) | group | group | 6 | 94 | 95 | 96 | 97 | 98 | 99 | 100 | 101 | 102 | 103 |
| 10 d | 6.00 | 26.75 | 24.00 | 25.50 | 25.00 | 26.00 | 26.70 | 25.00 | 25.00 | 26.50 | 25.00 | 26.00 | 25.50 |
| 15 d | 6.50 | 29.00 | 23.17 | 20.50 | 19.00 | 20.00 | 20.00 | 17.50 | 21.50 | 21.50 | 18.70 | 18.70 | 18.90 |
| 20 d | 5.50 | 35.33 | 19.50 | 16.80 | 15.80 | 15.00 | 11.50 | 11.00 | 16.70 | 15.80 | 15.30 | 11.70 | 11.00 |

### Embodiments 105 to 109: A Preparation Including Netarsudil Dimesylate, Timolol Mesylate and Travoprost

TABLE 167
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 105 | | 106 | | 107 | | 108 | | 109 | |
| 1. | Netarsudil dimesylate | 14.2 | mg | 14.2 | mg | 28.5 | mg | 28.5 | mg | 57.0 | mg |
| 2. | Timolol mesylate | 325 | mg | 650 | mg | 325 | mg | 650 | mg | 1300 | mg |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 168
Contents of various main medicine components in Embodiments
105 to 109 after being stored at 5° C. for 24 months

| | | Embodiment | Embodiment | Embodiment | Embodiment | Embodiment |
|---|---|---|---|---|---|---|
| | | 105 | 106 | 107 | 108 | 109 |
| No. | Component | Content % | | | | |
| 1. | Netarsudil dimesylate | 99.9 | 99.7 | 100.2 | 100.1 | 99.6 |
| 2. | Timolol mesylate | 101.3 | 100.2 | 100.4 | 99.8 | 99.7 |
| 3. | Travoprost | 100.2 | 99.8 | 99.6 | 99.2 | 100.5 |

TABLE 169
Contents of various main medicine components in commercially
available eye drops (Timoptic ®, Rhopress ®, and
Travatan ®) after being stored at 5° C. for 24 months

| | | Timoptic ® | Rhopress ® | Travatan ® |
|---|---|---|---|---|
| No. | Component | Content % | | |
| 1. | Timolol maleate | 99.7 | — | — |
| 2. | Netarsudil dimesylate | — | 99.9 | — |
| 3. | Travoprost | — | — | 99.8 |

According to Embodiments 105 to 109, the preparation including netarsudil dimesylate, timolol mesylate and travoprost has good stability after being stored under a storage condition of 5° C. for 24 months, which meets a requirement for a storage condition of the commercially available eye drops (Timoptic®, Rhopress®, and Travatan®) before opening.

TABLE 170
Contents of various main medicine components in Embodiments
105 to 109 after being stored at 25° C. for 6 weeks

| | | Embodiment | Embodiment | Embodiment | Embodiment | Embodiment |
|---|---|---|---|---|---|---|
| | | 105 | 106 | 107 | 108 | 109 |
| No. | Component | Content % | | | | |
| 1. | Netarsudil dimesylate | 99.8 | 98.9 | 101.4 | 100.4 | 99.7 |
| 2. | Timolol mesylate | 100.3 | 99.4 | 100.6 | 98.9 | 100.6 |
| 3. | Travoprost | 100.9 | 100.1 | 102.4 | 99.6 | 100.1 |

TABLE 171
Contents of various main medicine components in commercially
available eye drops (Timoptic ®, Rhopress ®, and
Travatan ®) after being stored at 25° C. for 6 weeks

| | | Timoptic ® | Rhopress ® | Travatan ® |
|---|---|---|---|---|
| No. | Component | Content % | | |
| 1. | Timolol maleate | 99.5 | — | — |
| 2. | Netarsudil dimesylate | — | 99.8 | — |
| 3. | Travoprost | — | — | 99.6 |

According to Embodiments 105 to 109, the preparation including netarsudil dimesylate, timolol mesylate and travoprost has good stability after being stored under a storage condition of 25° C. for 6 weeks, which meets a requirement for a storage condition of the commercially available eye drops (Timoptic®, Rhopress®, and Travatan®) after opening.

TABLE 172
Contents of various main medicine components in Embodiments
105 to 109 after being stored at 40° C. for 14 days

| | | Embodiment | Embodiment | Embodiment | Embodiment | Embodiment |
|---|---|---|---|---|---|---|
| | | 105 | 106 | 107 | 108 | 109 |
| No. | Component | Content % | | | | |
| 1. | Netarsudil dimesylate | 100.2 | 101.4 | 100.8 | 99.6 | 100.3 |
| 2. | Timolol mesylate | 100.1 | 100.5 | 99.5 | 99.6 | 99.1 |
| 3. | Travoprost | 99.1 | 99.2 | 100.1 | 100.8 | 100.1 |

TABLE 173
Contents of various main medicine components in commercially
available eye drops (Timoptic ®, Rhopress ®, and
Travatan ®) after being stored at 40° C. for 14 days

| | | Timoptic ® | Rhopress ® | Travatan ® |
|---|---|---|---|---|
| No. | Component | Content % | | |
| 1. | Timolol maleate | 99.8 | — | — |
| 2. | Netarsudil dimesylate | — | 99.5 | — |
| 3. | Travoprost | — | — | 99.7 |

According to Embodiments 105 to 109, the preparation including netarsudil dimesylate, timolol mesylate and travoprost has good stability after being stored under a storage condition of 40° C. for 14 days, which meets a requirement for departing from a storage condition of the commercially available eye drops (Timoptic®, Rhopress®, and Travatan®) for a short time.

### Embodiments 110 to 114: A Preparation Including Netarsudil Maleate, Timolol Maleate and Travoprost

TABLE 174
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 110 | | 111 | | 112 | | 113 | | 114 | |
| 1. | Netarsudil maleate | 12.6 | mg | 12.6 | mg | 25.1 | mg | 25.1 | mg | 50.2 | mg |
| 2. | Timolol maleate | 341.7 | mg | 683.5 | mg | 341.7 | mg | 683.5 | mg | 1367 | mg |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 175
Contents of various main medicine components in Embodiments
110 to 114 after being stored at 5° C. for 24 months

| | | Embodiment | Embodiment | Embodiment | Embodiment | Embodiment |
|---|---|---|---|---|---|---|
| | | 110 | 111 | 112 | 113 | 114 |
| No. | Component | Content % | | | | |
| 1. | Netarsudil maleate | 100.3 | 100.3 | 99.6 | 99.7 | 99.6 |
| 2. | Timolol maleate | 99.8 | 100.7 | 99.4 | 100.3 | 100.1 |
| 3. | Travoprost | 100.2 | 100.1 | 99.3 | 99.9 | 99.1 |

According to Embodiments 110 to 114, the preparation including netarsudil maleate, timolol maleate and travoprost has good stability after being stored under a storage condition of 5° C. for 24 months, which meets a requirement for a storage condition of the commercially available eye drops (Timoptic®, Rhopress®, and Travatan®) before opening.

TABLE 176
Contents of various main medicine components in Embodiments
110 to 114 after being stored at 25° C. for 6 weeks

| | | Embodiment | Embodiment | Embodiment | Embodiment | Embodiment |
|---|---|---|---|---|---|---|
| | | 110 | 111 | 112 | 113 | 114 |
| No. | Component | Content % | | | | |
| 1. | Netarsudil maleate | 100.1 | 100.8 | 99.5 | 99.8 | 100.5 |
| 2. | Timolol maleate | 99.3 | 99.3 | 100.5 | 100.4 | 100.1 |
| 3. | Travoprost | 99.2 | 100.1 | 100.7 | 99.1 | 98.9 |

According to Embodiments 110 to 114, the preparation including netarsudil maleate, timolol maleate and travoprost has good stability after being stored under a storage condition of 25° C. for 6 weeks, which meets a requirement for a storage condition of the commercially available eye drops (Timoptic®, Rhopress®, and Travatan®) after opening.

TABLE 177
Contents of various main medicine components in Embodiments
110 to 114 after being stored at 40° C. for 14 days

| | | Embodiment | Embodiment | Embodiment | Embodiment | Embodiment |
|---|---|---|---|---|---|---|
| | | 110 | 111 | 112 | 113 | 114 |
| No. | Component | Content % | | | | |
| 1. | Netarsudil maleate | 100.1 | 98.9 | 100.9 | 100.1 | 98.8 |
| 2. | Timolol maleate | 100.9 | 100.8 | 100.5 | 99.9 | 100.1 |
| 3. | Travoprost | 99.3 | 99.1 | 100.1 | 99.5 | 100.9 |

According to Embodiments 110 to 114, the preparation including netarsudil maleate, timolol maleate and travoprost has good stability after being stored under a storage condition of 40° C. for 14 days, which meets a requirement for departing from a storage condition of the commercially available eye drops (Timoptic®, Rhopress®, and Travatan®) for a short time.

### Embodiment 115: Evaluation of Pharmaceutical Effects of the Ophthalmic Medicine Composition Solutions in Embodiments 105 to 114 by Using a Japanese White Rabbit Glaucoma Model

### Experimental Grouping

Normal control group: no processing.
Model group: an intraocular hypertension model is established, and no eye drop is administered.
Treatment group 7: an intraocular hypertension model is established, and commercially available travoprost and timolol maleate eye drops (DuoTrav®) are administered.
Embodiment group 105: an intraocular hypertension model is established, and an ophthalmic medicine composition solution in Embodiment 105 is administered.
Embodiment group 106: an intraocular hypertension model is established, and an ophthalmic medicine composition solution in Embodiment 106 is administered.
Embodiment group 107: an intraocular hypertension model is established, and an ophthalmic medicine composition solution in Embodiment 107 is administered.
Embodiment group 108: an intraocular hypertension model is established, and an ophthalmic medicine composition solution in Embodiment 108 is administered.
Embodiment group 109: an intraocular hypertension model is established, and an ophthalmic medicine composition solution in Embodiment 109 is administered.
Embodiment group 110: an intraocular hypertension model is established, and an ophthalmic medicine composition solution in Embodiment 110 is administered.
Embodiment group 111: an intraocular hypertension model is established, and an ophthalmic medicine composition solution in Embodiment 111 is administered.
Embodiment group 112: an intraocular hypertension model is established, and an ophthalmic medicine composition solution in Embodiment 112 is administered.
Embodiment group 113: an intraocular hypertension model is established, and an ophthalmic medicine composition solution in Embodiment 113 is administered.
Embodiment group 114: an intraocular hypertension model is established, and an ophthalmic medicine composition solution in Embodiment 114 is administered.

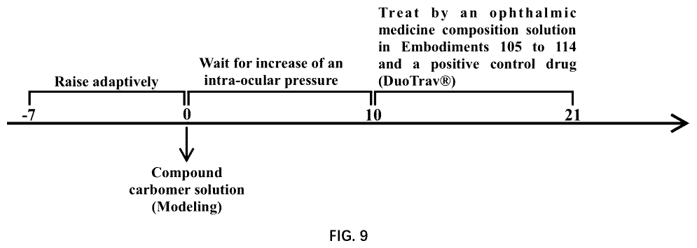

A dosage regimen: according to a current clinical dosage regimen of commercially available travoprost and timolol maleate eye drops (DuoTrav®), this experiment is mainly divided into the following groups: (1) the treatment group 7: 50 μL the commercially available travoprost and timolol maleate eye drops (DuoTrav®) are administered to each eye each time once a day (9:00 PM); and (2) Embodiment groups 105 to 114: 50 μL eye drops in Embodiments 105 to 114 are administered to each eye each time once a day (9:00 PM). Drug administration is started in all the groups on the tenth day when the models are established successfully and lasts for 10 days. No processing is made to the normal control group and the model group. The dosage regimen is shown in FIG. 9.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 7 and Embodiment groups 105, 106, 107, 110, 111 and 112, the IOP can be reduced significantly by the ophthalmic composition solutions of Embodiment groups 108, 109, 113 and 114, however, IOP actual measured values and IOP variation values in Embodiment groups 108, 109, 113 and 114 have no significant difference.

TABLE 178
Difference values between intra-ocular pressure actual measured values of each group of rabbits measured on the tenth
day, the fifteenth day and the twentieth day and an initial intra-ocular pressure (the intra-ocular pressure actual
measured value on the tenth day) when drug administration is performed for 10 days on end starting with the tenth day

| | IOP variation value (mmHg) | | | | | | | | | | | | |
|---|---|---|---|---|---|---|---|---|---|---|---|---|---|
| | | | Treat- | Embo- | Embo- | Embo- | Embo- | Embo- | Embo- | Embo- | Embo- | Embo- | Embo- |
| | Normal | | ment | diment | diment | diment | diment | diment | diment | diment | diment | diment | diment |
| Time | control | Model | group | group | group | group | group | group | group | group | group | group | group |
| (d) | group | group | 7 | 105 | 106 | 107 | 108 | 109 | 110 | 111 | 112 | 113 | 114 |
| 10 d | — | — | — | — | — | — | — | — | — | — | — | — | — |
| 15 d | 0.50 | 2.25 | −4.00 | −5.50 | −5.00 | −5.30 | −7.70 | −5.30 | −5.40 | −5.50 | −5.70 | −8.20 | −10.50 |
| 20 d | −0.50 | 8.58 | −5.70 | −11.00 | −10.70 | −12.00 | −16.20 | −16.00 | −10.70 | −12.30 | −12.10 | −16.40 | −16.90 |

TABLE 179
Intra-ocular pressure actual measured values of each group of rabbits measured on the tenth day, the fifteenth
day and the twentieth day when drug administration is performed for 10 days on end starting with the tenth day

| | IOP actual measured value (mmHg) | | | | | | | | | | | | |
|---|---|---|---|---|---|---|---|---|---|---|---|---|---|
| | | | Treat- | Embo- | Embo- | Embo- | Embo- | Embo- | Embo- | Embo- | Embo- | Embo- | Embo- |
| | Normal | | ment | diment | diment | diment | diment | diment | diment | diment | diment | diment | diment |
| Time | control | Model | group | group | group | group | group | group | group | group | group | group | group |
| (d) | group | group | 7 | 105 | 106 | 107 | 108 | 109 | 110 | 111 | 112 | 113 | 114 |
| 10 d | 6.00 | 26.75 | 25.00 | 26.00 | 25.00 | 26.00 | 26.70 | 26.00 | 25.70 | 26.50 | 26.00 | 26.70 | 27.00 |
| 15 d | 6.50 | 29.00 | 21.00 | 20.50 | 20.00 | 20.70 | 19.00 | 20.70 | 20.30 | 21.00 | 20.30 | 18.50 | 16.50 |
| 20 d | 5.50 | 35.33 | 19.30 | 15.00 | 14.30 | 14.00 | 10.50 | 10.00 | 15.00 | 14.20 | 13.90 | 10.30 | 10.10 |

### Embodiments 116 to 120: A Preparation Including Netarsudil Dimesylate, Timolol Mesylate and Tafluprost

TABLE 180
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 116 | | 117 | | 118 | | 119 | | 120 | |
| 1. | Netarsudil dimesylate | 14.2 | mg | 14.2 | mg | 28.5 | mg | 28.5 | mg | 57.0 | mg |
| 2. | Timolol mesylate | 325 | mg | 650 | mg | 325 | mg | 650 | mg | 1300 | mg |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 181
Contents of various main medicine components in Embodiments
116 to 120 after being stored at 5° C. for 24 months

| | | Embodiment | Embodiment | Embodiment | Embodiment | Embodiment |
|---|---|---|---|---|---|---|
| | | 116 | 117 | 118 | 119 | 120 |
| No. | Component | Content % | | | | |
| 1. | Netarsudil dimesylate | 99.3 | 99.6 | 100.7 | 100.4 | 99.1 |
| 2. | Timolol mesylate | 100.3 | 100.2 | 100.5 | 99.5 | 99.8 |
| 3. | Tafluprost | 101.2 | 100.8 | 99.7 | 99.3 | 99.5 |

TABLE 182
Contents of various main medicine components in commercially
available eye drops (Timoptic ®, Rhopress ®, Tapros ®) after
being stored at 5° C. for 24 months

| | | Timoptic ® | Rhopress ® | Tapros ® |
|---|---|---|---|---|
| No. | Component | Content % | | |
| 1. | Timolol maleate | 99.7 | — | — |
| 2. | Netarsudil dimesylate | — | 99.9 | — |
| 3. | Tafluprost | — | — | 99.4 |

According to Embodiments 116 to 120, the preparation including netarsudil dimesylate, timolol mesylate and tafluprost has good stability after being stored under a storage condition of 5° C. for 24 months, which meets a requirement for a storage condition of the commercially available eye drops (Timoptic®, Rhopress®, and Tapros®) before opening.

TABLE 183
Contents of various main medicine components in Embodiments
116 to 120 after being stored at 25° C. for 6 weeks

| | | Embodiment | Embodiment | Embodiment | Embodiment | Embodiment |
|---|---|---|---|---|---|---|
| | | 116 | 117 | 118 | 119 | 120 |
| No. | Component | Content % | | | | |
| 1. | Netarsudil dimesylate | 99.1 | 99.4 | 100.4 | 100.3 | 99.4 |
| 2. | Timolol mesylate | 100.3 | 99.6 | 99.6 | 99.9 | 99.6 |
| 3. | Tafluprost | 100.2 | 99.1 | 101.4 | 99.7 | 100.5 |

TABLE 184
Contents of various main medicine components in commercially
available eye drops (Timoptic ®, Rhopress ®, and
Tapros ®) after being stored at 25° C. for 6 weeks

| | | Timoptic ® | Rhopress ® | Tapros ® |
|---|---|---|---|---|
| No. | Component | Content % | | |
| 1. | Timolol maleate | 99.5 | — | — |
| 2. | Netarsudil dimesylate | — | 99.8 | — |
| 3. | Tafluprost | — | — | 99.3 |

According to Embodiments 116 to 120, the preparation including netarsudil dimesylate, timolol mesylate and tafluprost has good stability after being stored under a storage condition of 25° C. for 6 weeks, which meets a requirement for a storage condition of the commercially available eye drops (Timoptic®, Rhopress®, and Tapros®) after opening.

TABLE 185
Contents of various main medicine components in Embodiments
116 to 120 after being stored at 40° C. for 14 days

| | | Embodiment | Embodiment | Embodiment | Embodiment | Embodiment |
|---|---|---|---|---|---|---|
| | | 116 | 117 | 118 | 119 | 120 |
| No. | Component | Content % | | | | |
| 1. | Netarsudil maleate | 100.8 | 100.4 | 99.8 | 100.6 | 100.4 |
| 2. | Timolol maleate | 100.7 | 100.4 | 99.7 | 99.4 | 100.1 |
| 3. | Tafluprost | 99.5 | 99.9 | 99.4 | 99.8 | 100.2 |

TABLE 186
Contents of various main medicine components in commercially
available eye drops (Timoptic ®, Rhopress ®, and
Tapros ®) after being stored at 40° C. for 14 days

| | | Timoptic ® | Rhopress ® | Tapros ® |
|---|---|---|---|---|
| No. | Component | Content % | | |
| 1. | Timolol maleate | 99.8 | — | — |
| 2. | Netarsudil dimesylate | — | 99.5 | — |
| 3. | Tafluprost | — | — | 99.8 |

According to Embodiments 116 to 120, the preparation including netarsudil dimesylate, timolol mesylate and tafluprost has good stability after being stored under a storage condition of 40° C. for 14 days, which meets a requirement for departing from a storage condition of the commercially available eye drops (Timoptic®, Rhopress®, and Tapros®) for a short time.

### Embodiments 121 to 125: A Preparation Including Netarsudil Maleate, Timolol Maleate and Tafluprost

TABLE 187
Preparation of a local ophthalmic medicine composition solution
for reducing an intra-ocular pressure as follows:

| | | Embodiment | | Embodiment | | Embodiment | | Embodiment | | Embodiment | |
|---|---|---|---|---|---|---|---|---|---|---|---|
| No. | Component | 121 | | 122 | | 123 | | 124 | | 125 | |
| 1. | Netarsudil maleate | 12.6 | mg | 12.6 | mg | 25.1 | mg | 25.1 | mg | 50.2 | mg |
| 2. | Timolol maleate | 341.7 | mg | 683.5 | mg | 341.7 | mg | 683.5 | mg | 1367 | mg |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 188
Contents of various main medicine components in Embodiments
121 to 125 after being stored at 5° C. for 24 months

| | | Embodiment | Embodiment | Embodiment | Embodiment | Embodiment |
|---|---|---|---|---|---|---|
| | | 121 | 122 | 123 | 124 | 125 |
| No. | Component | Content % | | | | |
| 1. | Netarsudil maleate | 100.1 | 100.4 | 100.5 | 99.5 | 99.2 |
| 2. | Timolol maleate | 100.8 | 99.3 | 99.4 | 100.1 | 100.3 |
| 3. | Tafluprost | 101.2 | 100.1 | 99.3 | 99.4 | 100.1 |

According to Embodiments 121 to 125, the preparation including netarsudil maleate, timolol maleate and tafluprost has good stability after being stored under a storage condition of 5° C. for 24 months, which meets a requirement for a storage condition of the commercially available eye drops (Timoptic®, Rhopress®, and Tapro®) before opening.

TABLE 189
Contents of various main medicine components in Embodiments
121 to 125 after being stored at 25° C. for 6 weeks

| | | Embodiment | Embodiment | Embodiment | Embodiment | Embodiment |
|---|---|---|---|---|---|---|
| | | 121 | 122 | 123 | 124 | 125 |
| No. | Component | Content % | | | | |
| 1. | Netarsudil maleate | 100.1 | 100.8 | 99.5 | 99.8 | 100.5 |
| 2. | Timolol maleate | 99.3 | 99.3 | 100.5 | 100.4 | 100.1 |
| 3. | Tafluprost | 99.2 | 100.1 | 100.7 | 99.1 | 98.9 |

According to Embodiments 121 to 125, the preparation including netarsudil maleate, timolol maleate and tafluprost has good stability after being stored under a storage condition of 25° C. for 6 weeks, which meets a requirement for a storage condition of the commercially available eye drops (Timoptic®, Rhopress®, and Tapros®) after opening.

TABLE 190
Contents of various main medicine components in Embodiments
121 to 125 after being stored at 40° C. for 14 days

| | | Embodiment | Embodiment | Embodiment | Embodiment | Embodiment |
|---|---|---|---|---|---|---|
| | | 121 | 122 | 123 | 124 | 125 |
| No. | Component | Content % | | | | |
| 1. | Netarsudil maleate | 100.1 | 98.9 | 100.9 | 100.1 | 98.8 |
| 2. | Timolol maleate | 100.9 | 100.8 | 100.5 | 99.9 | 100.1 |
| 3. | Tafluprost | 99.3 | 99.1 | 100.1 | 99.5 | 100.9 |

According to Embodiments 121 to, the preparation including netarsudil maleate, timolol maleate and tafluprost has good stability after being stored under a storage condition of 40° C. for 14 days, which meets a requirement for departing from a storage condition of the commercially available eye drops (Timoptic®, Rhopress®, and Tapros®) for a short time.

### Embodiment 126: Evaluation of Pharmaceutical Effects of the Ophthalmic Medicine Composition Solutions in Embodiments 116 to 125 by Using a Japanese White Rabbit Glaucoma Model

### Experimental Grouping

Normal control group: no processing.
Model group: an intraocular hypertension model is established, and no eye drop is administered.
Treatment group 8: an intraocular hypertension model is established, and commercially available tafluprost and timolol maleate eye drops (Tapcom®) are administered.
Embodiment group 116: an intraocular hypertension model is established, and an ophthalmic medicine composition solution in Embodiment 116 is administered.
Embodiment group 117: an intraocular hypertension model is established, and an ophthalmic medicine composition solution in Embodiment 117 is administered.
Embodiment group 118: an intraocular hypertension model is established, and an ophthalmic medicine composition solution in Embodiment 118 is administered.
Embodiment group 119: an intraocular hypertension model is established, and an ophthalmic medicine composition solution in Embodiment 119 is administered.
Embodiment group 120: an intraocular hypertension model is established, and an ophthalmic medicine composition solution in Embodiment 120 is administered.
Embodiment group 121: an intraocular hypertension model is established, and an ophthalmic medicine composition solution in Embodiment 121 is administered.
Embodiment group 122: an intraocular hypertension model is established, and an ophthalmic medicine composition solution in Embodiment 122 is administered.
Embodiment group 123: an intraocular hypertension model is established, and an ophthalmic medicine composition solution in Embodiment 123 is administered.
Embodiment group 124: an intraocular hypertension model is established, and an ophthalmic medicine composition solution in Embodiment 124 is administered.
Embodiment group 125: an intraocular hypertension model is established, and an ophthalmic medicine composition solution in Embodiment 125 is administered.

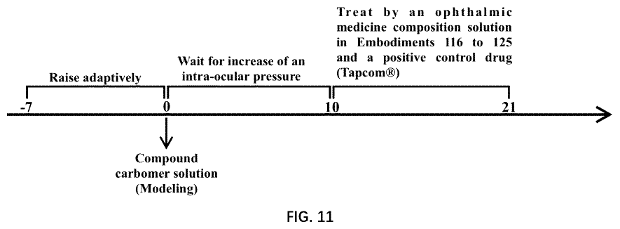

A dosage regimen: according to a current clinical dosage regimen of commercially available tafluprost and timolol maleate eye drops (Tapcom®), this experiment is mainly divided into the following groups: (1) the treatment group 8: 50 μL the commercially available tafluprost and timolol maleate eye drops (Tapcom®) are administered to each eye each time once a day (9:00 PM); and (2) Embodiment groups 116 to 125: 50 μL eye drops in Embodiments 116 to 125 are administered to each eye each time once a day (9:00 PM). Drug administration is started in all the groups on the tenth day when the models are established successfully and lasts for 10 days. No processing is made to the normal control group and the model group. The dosage regimen is shown in FIG. 11.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 8 and Embodiment groups 116, 117, 118, 121, 122 and 123, the IOP can be reduced significantly by the ophthalmic composition solutions of Embodiment groups 119, 120, 124 and 125, however, IOP actual measured values and IOP variation values in Embodiment groups 119, 120, 124 and 125 have no significant difference.

TABLE 191
Difference values between intra-ocular pressure actual measured values of each group of rabbits measured on the tenth
day, the fifteenth day and the twentieth day and an initial intra-ocular pressure (the intra-ocular pressure actual
measured value on the tenth day) when drug administration is performed for 10 days on end starting with the tenth day

| | IOP variation value (mmHg) | | | | | | | | | | | | |
|---|---|---|---|---|---|---|---|---|---|---|---|---|---|
| | | | Treat- | Embo- | Embo- | Embo- | Embo- | Embo- | Embo- | Embo- | Embo- | Embo- | Embo- |
| | Normal | | ment | diment | diment | diment | diment | diment | diment | diment | diment | diment | diment |
| Time | control | Model | group | group | group | group | group | group | group | group | group | group | group |
| (d) | group | group | 8 | 116 | 117 | 118 | 119 | 120 | 121 | 122 | 123 | 124 | 125 |
| 10 d | — | — | — | — | — | — | — | — | — | — | — | — | — |
| 15 d | 0.50 | 2.25 | −5.70 | −6.00 | −5.00 | −4.70 | −8.00 | −6.70 | −7.20 | −5.30 | −6.80 | −8.70 | −11.00 |
| 20 d | −0.50 | 8.58 | −7.30 | −11.30 | −11.70 | −11.80 | −16.20 | −15.30 | −11.30 | −12.80 | −12.00 | −16.10 | −16.80 |

TABLE 192
Intra-ocular pressure actual measured values of each group of rabbits measured on the tenth day, the fifteenth
day and the twentieth day when drug administration is performed for 10 days on end starting with the tenth day

| | IOP actual measured value (mmHg) | | | | | | | | | | | | |
|---|---|---|---|---|---|---|---|---|---|---|---|---|---|
| | | | Treat- | Embo- | Embo- | Embo- | Embo- | Embo- | Embo- | Embo- | Embo- | Embo- | Embo- |
| | Normal | | ment | diment | diment | diment | diment | diment | diment | diment | diment | diment | diment |
| Time | control | Model | group | group | group | group | group | group | group | group | group | group | group |
| (d) | group | group | 8 | 116 | 117 | 118 | 119 | 120 | 121 | 122 | 123 | 124 | 125 |
| 10 d | 6.00 | 26.75 | 26.00 | 26.50 | 26.30 | 26.00 | 27.00 | 26.00 | 26.50 | 27.00 | 26.50 | 27.00 | 27.30 |
| 15 d | 6.50 | 29.00 | 20.30 | 20.50 | 21.30 | 21.30 | 19.00 | 19.30 | 19.30 | 21.70 | 19.70 | 18.30 | 16.30 |
| 20 d | 5.50 | 35.33 | 18.70 | 15.20 | 14.60 | 14.20 | 10.80 | 10.70 | 15.20 | 14.20 | 14.50 | 10.90 | 10.50 |

### Embodiment 127: A Preparation Including Netarsudil Dimesylate, Timolol Hydrobromide and Latanoprost

TABLE 193
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol hydrobromide | 628 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 194
Contents of various main medicine components in
Embodiment 127 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.4 | 100.4 |
| 2. | Timolol hydrobromide | 100.1 | 99.6 |
| 3. | Latanoprost | 100.3 | 99.5 |

According to Embodiment 127, the preparation including netarsudil dimesylate, timolol hydrobromide and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 128: A Preparation Including Netarsudil Dimesylate, Timolol Sulfate and Latanoprost

TABLE 195
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol sulfate | 655 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s | |
| 8. | Water for injection added by | 100 | mL |

A preparation process may refer to the preparation process in Embodiments 83 to 87.

TABLE 196
Contents of various main medicine components in
Embodiment 128 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.2 | 100.5 |
| 2. | Timolol sulfate | 100.5 | 100.1 |
| 3. | Latanoprost | 100.4 | 100.6 |

According to Embodiment 128, the preparation including netarsudil dimesylate, timolol sulfate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiments 129: A Preparation Including Netarsudil Dimesylate, Timolol Esilate and Latanoprost

TABLE 197
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol esilate | 674 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 198
Contents of various main medicine components in
Embodiment 129 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.8 | 100.6 |
| 2. | Timolol esilate | 100.1 | 100.3 |
| 3. | Latanoprost | 100.4 | 100.1 |

According to Embodiment 129, the preparation including netarsudil dimesylate, timolol esilate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 130: A Preparation Including Netarsudil Dimesylate, Timolol Nitrate and Latanoprost

TABLE 199
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol nitrate | 600 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 200
Contents of various main medicine components in
Embodiment 130 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.3 | 100.4 |
| 2. | Timolol nitrate | 99.4 | 99.8 |
| 3. | Latanoprost | 100.6 | 100.2 |

According to Embodiment 130, the preparation including netarsudil dimesylate, timolol nitrate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 131: A Preparation Including Netarsudil Dimesylate, Timolol Citrate and Latanoprost

TABLE 201
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol citrate | 804 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 202
Contents of various main medicine components in
Embodiment 131 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.2 | 100.4 |
| 2. | Timolol citrate | 100.5 | 99.8 |
| 3. | Latanoprost | 100.7 | 99.5 |

According to Embodiment 131, the preparation including netarsudil dimesylate, timolol citrate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 132: A Preparation Including Netarsudil Dimesylate, Timolol Tartrate and Latanoprost

TABLE 203
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol tartrate | 737 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 204
Contents of various main medicine components in
Embodiment 132 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.6 | 100.1 |
| 2. | Timolol tartrate | 100.6 | 99.9 |
| 3. | Latanoprost | 100.1 | 100.3 |

According to Embodiment 132, the preparation including netarsudil dimesylate, timolol tartrate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 133: A Preparation Including Netarsudil Dimesylate, Timolol Salicylate and Latanoprost

TABLE 205
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol salicylate | 718 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may re er to the preparation process in Embodiments 83 to 87.

TABLE 206
Contents of various main medicine components in
Embodiment 133 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.5 | 100.1 |
| 2. | Timolol salicylate | 99.4 | 99.8 |
| 3. | Latanoprost | 100.3 | 100.4 |

According to Embodiment 133, the preparation including netarsudil dimesylate, timolol salicylate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 134: A Preparation Including Netarsudil Dimesylate, Timolol Malate and Latanoprost

TABLE 207
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol malate | 712 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 208
Contents of various main medicine components in
Embodiment 134 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.3 | 100.1 |
| 2. | Timolol malate | 100.1 | 100.5 |
| 3. | Latanoprost | 100.7 | 100.8 |

According to Embodiment 134, the preparation including netarsudil dimesylate, timolol malate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 135: A Preparation Including Netarsudil Dimesylate, Timolol Lactate and Latanoprost

TABLE 209
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol lactate | 642 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 210
Contents of various main medicine components in
Embodiment 135 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.8 | 100.4 |
| 2. | Timolol lactate | 100.3 | 100.5 |
| 3. | Latanoprost | 99.9 | 100.2 |

According to Embodiment 135, the preparation including netarsudil dimesylate, timolol lactate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 136: A Preparation Including Netarsudil Dimesylate, Timolol Phenylacetate and Latanoprost

TABLE 211
Preparation of a local ophthalmic medicine composition solution
for reducing an intra-ocular pressure as follows:

| No. | Component | Weight | |
|---|---|---|---|
| 1. | Netarsudil dimesylate | 28.5 | mg |
| 2. | Timolol phenylacetate | 715 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 212
Contents of various main medicine components in
Embodiment 136 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.3 | 100.1 |
| 2. | Timolol phenylacetate | 100.3 | 100.6 |
| 3. | Latanoprost | 100.6 | 100.5 |

According to Embodiment 136, the preparation including netarsudil dimesylate, timolol phenylacetate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 137: A Preparation Including Netarsudil Dihydrobromide, Timolol Maleate and Latanoprost

TABLE 213
Preparation of a local ophthalmic medicine composition solution
for reducing an intra-ocular pressure as follows:

| No. | Component | Weight | |
|---|---|---|---|
| 1. | Netarsudil dihydrobromide | 27.1 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 214
Contents of various main medicine components in
Embodiment 137 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dihydrobromide | 100.4 | 100.1 |
| 2. | Timolol maleate | 99.5 | 100.4 |
| 3. | Latanoprost | 100.5 | 100.7 |

According to Embodiment 137, the preparation including netarsudil dihydrobromide, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 138: A Preparation Including Netarsudil Dihydrochloride, Timolol Maleate and Latanoprost

TABLE 215
Preparation of a local ophthalmic medicine composition solution
for reducing an intra-ocular pressure as follows:

| No. | Component | Weight | |
|---|---|---|---|
| 1. | Netarsudil dihydrochloride | 23.2 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 216
Contents of various main medicine components in
Embodiment 138 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dihydrochloride | 100.5 | 100.6 |
| 2. | Timolol maleate | 100.3 | 99.8 |
| 3. | Latanoprost | 100.4 | 100.3 |

According to Embodiment 138, the preparation including netarsudil dihydrochloride, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 139: A Preparation Including Netarsudil Sulfate, Timolol Maleate and Latanoprost

TABLE 217
Preparation of a local ophthalmic medicine composition solution
for reducing an intra-ocular pressure as follows:

| No. | Component | Weight | |
|---|---|---|---|
| 1. | Netarsudil sulfate | 24.3 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 218
Contents of various main medicine components in
Embodiment 139 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil sulfate | 99.5 | 100.1 |
| 2. | Timolol maleate | 100.2 | 100.5 |
| 3. | Latanoprost | 100.3 | 100.1 |

According to Embodiment 139, the preparation including netarsudil sulfate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 140: A Preparation Including Netarsudil Diformate, Timolol Maleate and Latanoprost

TABLE 219
Preparation of a local ophthalmic medicine composition solution
for reducing an intra-ocular pressure as follows:

| No. | Component | Weight | |
|---|---|---|---|
| 1. | Netarsudil diformate | 24.1 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 220
Contents of various main medicine components in
Embodiment 140 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diformate | 100.6 | 100.2 |
| 2. | Timolol maleate | 100.6 | 100.7 |
| 3. | Latanoprost | 100.4 | 100.2 |

According to Embodiment 140, the preparation including netarsudil diformate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 141: A Preparation Including Netarsudil Dinitrate, Timolol Maleate and Latanoprost

TABLE 221
Preparation of a local ophthalmic medicine composition solution
for reducing an intra-ocular pressure as follows:

| No. | Component | Weight | |
|---|---|---|---|
| 1. | Netarsudil dinitrate | 25.6 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 222
Contents of various main medicine components in
Embodiment 141 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dinitrate | 100.3 | 100.2 |
| 2. | Timolol maleate | 100.5 | 100.1 |
| 3. | Latanoprost | 100.6 | 100.4 |

According to Embodiment 141, the preparation including netarsudil dinitrate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 142: A Preparation Including Netarsudil Diacetate, Timolol Maleate and Latanoprost

TABLE 223
Preparation of a local ophthalmic medicine composition solution
for reducing an intra-ocular pressure as follows:

| No. | Component | Weight | |
|---|---|---|---|
| 1. | Netarsudil diacetate | 27.8 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 224
Contents of various main medicine components in
Embodiment 142 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diacetate | 100.8 | 100.5 |
| 2. | Timolol maleate | 100.3 | 100.1 |
| 3. | Latanoprost | 100.4 | 100.5 |

According to Embodiment 142, the preparation including netarsudil diacetate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 143: A Preparation Including Netarsudil Dibenzoate, Timolol Maleate and Latanoprost

TABLE 225
Preparation of a local ophthalmic medicine composition solution
for reducing an intra-ocular pressure as follows:

| No. | Component | Weight | |
|---|---|---|---|
| 1. | Netarsudil dibenzoate | 30.8 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 226
Contents of various main medicine components in
Embodiment 143 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dibenzoate | 100.5 | 99.9 |
| 2. | Timolol maleate | 100.1 | 100.2 |
| 3. | Latanoprost | 99.8 | 100.1 |

According to Embodiment 143, the preparation including netarsudil dibenzoate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 144: A Preparation Including Netarsudil Oxalate, Timolol Maleate and Latanoprost

TABLE 227
Preparation of a local ophthalmic medicine composition solution
for reducing an intra-ocular pressure as follows:

| No. | Component | Weight | |
|---|---|---|---|
| 1. | Netarsudil oxalate | 27.9 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 228
Contents of various main medicine components in
Embodiment 144 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil oxalate | 100.4 | 100.2 |
| 2. | Timolol maleate | 99.4 | 99.8 |
| 3. | Latanoprost | 100.4 | 100.2 |

According to Embodiment 144, the preparation including netarsudil oxalate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 145: A Preparation Including Netarsudil Succinate, Timolol Maleate and Latanoprost

TABLE 229
Preparation of a local ophthalmic medicine composition solution
for reducing an intra-ocular pressure as follows:

| No. | Component | Weight | |
|---|---|---|---|
| 1. | Netarsudil succinate | 25.2 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 230
Contents of various main medicine components in
Embodiment 145 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil succinate | 100.4 | 100.2 |
| 2. | Timolol maleate | 100.2 | 99.8 |
| 3. | Latanoprost | 100.4 | 99.5 |

According to Embodiment 145, the preparation including netarsudil succinate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Embodiment 146: A Preparation Including Netarsudil Diphenylacetate, Timolol Maleate and Latanoprost

TABLE 231
Preparation of a local ophthalmic medicine composition solution
for reducing an intra-ocular pressure as follows:

| No. | Component | Weight | |
|---|---|---|---|
| 1. | Netarsudil diphenylacetate | 32.0 | mg |
| 2. | Timolol maleate | 683.5 | mg |
| 3. | Latanoprost | 5 | mg |
| 4. | Boric acid | 50 | mg |
| 5. | Mannitol | 5000 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 232
Contents of various main medicine components in
Embodiment 146 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diphenylacetate | 100.1 | 100.3 |
| 2. | Timolol maleate | 99.4 | 100.2 |
| 3. | Latanoprost | 99.1 | 99.5 |

According to Embodiment 146, the preparation including netarsudil diphenylacetate, timolol maleate and latanoprost has good stability after being stored under a storage condition of 5° C. for 24 months, which meets a requirement for a storage condition of the commercially available eye drops (Timoptic®, Rhopress®, and Xalatan®) before opening; and has good stability after being stored under a storage condition of 25° C. for 6 weeks, which meets a requirement for a storage condition of the commercially available eye drops (Timoptic®, Rhopress®, and Xalatan®) after opening.

### Comparative Examples 49 to 52: A Preparation Including Netarsudil Dimesylate, Timolol Maleate and Latanoprost

TABLE 233
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 25 | | example 26 | | example 27 | | example 28 | |
| 1. | Netarsudil dimesylate | 14.2 | mg | 14.2 | mg | 28.5 | mg | 28.5 | mg |
| 2. | Timolol maleate | 341.7 | mg | 683.5 | mg | 341.7 | mg | 683.5 | mg |
| 3. | Latanoprost | 5 | mg | 5 | mg | 5 | mg | 5 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection | 100 | mL | 100 | mL | 100 | mL | 100 | mL |
| | added by | | | | | | | | |

A preparation process may refer to the preparation process in Embodiments 83 to 87.

TABLE 234
Contents of various main medicine components in Comparative
examples 49 to 52 after being stored at 5° C. for 7 days

| | | Comparative | Comparative | Comparative | Comparative |
|---|---|---|---|---|---|
| | | example 25 | example 26 | example 27 | example 28 |
| No. | Component | Content % | | | |
| 1. | Netarsudil dimesylate | <30 | <30 | <30 | <30 |
| 2. | Timolol maleate | 99.3 | 99.5 | 100.3 | 100.1 |
| 3. | Latanoprost | 100.2 | 100.3 | 99.8 | 100.3 |

According to Comparative examples 49 to 52, the preparation including netarsudil dimesylate, timolol maleate and latanoprost has incompatibility under the storage condition of 5° C., and netarsudil dimesylate is precipitated.

### Comparative Examples 53 to 56: A Preparation Including Netarsudil Dimesylate, Maleic Acid and Latanoprost

TABLE 235
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 29 | | example 30 | | example 31 | | example 32 | |
| 1. | Netarsudil dimesylate | 14.2 | mg | 14.2 | mg | 28.5 | mg | 28.5 | mg |
| 2. | Maleic acid | 91.8 | mg | 183.5 | mg | 91.8 | mg | 183.5 | mg |
| 3. | Latanoprost | 5 | mg | 5 | mg | 5 | mg | 5 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 236
Contents of various main medicine components in Comparative
examples 53 to 56 after being stored at 5° C. for 7 days

| | | Comparative | Comparative | Comparative | Comparative |
|---|---|---|---|---|---|
| | | example 29 | example 30 | example 31 | example 32 |
| No. | Component | Content % | | | |
| 1. | Netarsudil dimesylate | <30 | <30 | <30 | <30 |
| 2. | Latanoprost | 99.9 | 99.5 | 100.4 | 100.2 |

According to Comparative examples 53 to 56, the preparation including netarsudil dimesylate, maleic acid and latanoprost has incompatibility under the storage condition of 5° C., and netarsudil dimesylate is precipitated.

### Comparative Examples 33 to 36: A Preparation Including Netarsudil Dimesylate, Timolol Maleate and Bimatoprost

TABLE 237
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No | Component | example 33 | | example 34 | | example 35 | | example 36 | |
| 1. | Netarsudil dimesylate | 14.2 | mg | 14.2 | mg | 28.5 | mg | 28.5 | mg |
| 2. | Timolol maleate | 341.7 | mg | 683.5 | mg | 341.7 | mg | 683.5 | mg |
| 3. | Bimatoprost | 30 | mg | 30 | mg | 30 | mg | 30 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 238
Contents of various main medicine components in Comparative
examples 33 to 36 after being stored at 5° C. for 7 days

| | | Comparative | Comparative | Comparative | Comparative |
|---|---|---|---|---|---|
| | | example 33 | example 34 | example 35 | example 36 |
| No. | Component | Content % | | | |
| 1. | Netarsudil dimesylate | <30 | <30 | <30 | <30 |
| 2. | Timolol maleate | 99.7 | 99.6 | 100.1 | 99.5 |
| 3. | Bimatoprost | 100.1 | 100.3 | 99.8 | 100.3 |

According to Comparative examples 33 to 36, the preparation including netarsudil dimesylate, timolol maleate and bimatoprost has incompatibility under the storage condition of 5° C., and netarsudil dimesylate is precipitated.

### Comparative Examples 37 to 40: A Preparation Including Netarsudil Dimesylate, Maleic Acid and Bimatoprost

TABLE 239
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 37 | | example 38 | | example 39 | | example 40 | |
| 1. | Netarsudil dimesylate | 14.2 | mg | 14.2 | mg | 28.5 | mg | 28.5 | mg |
| 2. | Maleic acid | 91.8 | mg | 183.5 | mg | 91.8 | mg | 183.5 | mg |
| 3. | Bimatoprost | 30 | mg | 30 | mg | 30 | mg | 30 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 5 | mg | 5 | mg | 5 | mg | 5 | mg |
| 7. | Sodium hydroxide | q.s. | | q.s. | | q.s | | q.s. | |
| 8. | Water for injection added by | 100 | mL | 100 | mL | 100 | mL | 100 | mL |

A preparation process may refer to the preparation process in Embodiments 83 to 87.

TABLE 240
Contents of various main medicine components in Comparative
examples 37 to 40 after being stored at 5° C. for 7 days

| | | Comparative | Comparative | Comparative | Comparative |
|---|---|---|---|---|---|
| | | example 37 | example 38 | example 39 | example 40 |
| No. | Component | Content % | | | |
| 1. | Netarsudil dimesylate | <30 | <30 | <30 | <30 |
| 2. | Bimatoprost | 99.4 | 99.3 | 100.1 | 100.0 |

According to Comparative examples 37 to 40, the preparation including netarsudil dimesylate, maleic acid and bimatoprost has incompatibility under the storage condition of 5° C., and netarsudil dimesylate is precipitated.

### Comparative Examples 41 to 44: A Preparation Including Netarsudil Dimesylate, Timolol Maleate and Travoprost

TABLE 241
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 41 | | example 42 | | example 43 | | example 44 | |
| 1. | Netarsudil dimesylate | 14.2 | mg | 14.2 | mg | 28.5 | mg | 28.5 | mg |
| 2. | Timolol maleate | 341.7 | mg | 683.5 | mg | 341.7 | mg | 683.5 | mg |
| 3. | Travoprost | 4 | mg | 4 | mg | 4 | mg | 4 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 15 | mg | 15 | mg | 15 | mg | 15 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 242
Contents of various main medicine components in Comparative
examples 41 to 44 after being stored at 5° C. for 7 days

| | | Comparative | Comparative | Comparative | Comparative |
|---|---|---|---|---|---|
| | | example 41 | example 42 | example 43 | example 44 |
| No. | Component | Content % | | | |
| 1. | Netarsudil dimesylate | <30 | <30 | <30 | <30 |
| 2. | Timolol maleate | 99.2 | 99.7 | 99.1 | 99.6 |
| 3. | Travoprost | 100.4 | 100.2 | 99.9 | 100.1 |

According to Comparative examples 41 to 44, the preparation including netarsudil dimesylate, timolol maleate and travoprost has incompatibility under the storage condition of 5° C., and netarsudil dimesylate is precipitated.

### Comparative Examples 45 to 48: A Preparation Including Netarsudil Dimesylate, Maleic Acid and Travoprost

TABLE 243
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 45 | | example 46 | | example 47 | | example 48 | |
| 1. | Netarsudil dimesylate | 14.2 | mg | 14.2 | mg | 28.5 | mg | 28.5 | mg |
| 2. | Maleic acid | 91.8 | mg | 183.5 | mg | 91.8 | mg | 183.5 | mg |
| 3. | Travoprost | 4 | mg | 4 | mg | 4 | mg | 4 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 15 | mg | 15 | mg | 15 | mg | 15 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 244
Contents of various main medicine components in Comparative
examples 45 to 48 after being stored at 5° C. for 7 days

| | | Comparative | Comparative | Comparative | Comparative |
|---|---|---|---|---|---|
| | | example 45 | example 46 | example 47 | example 48 |
| No. | Component | Content % | | | |
| 1. | Netarsudil dimesylate | <30 | <30 | <30 | <30 |
| 2. | Travoprost | 99.2 | 100.3 | 100.9 | 100.4 |

According to Comparative examples 45 to 48, the preparation including netarsudil dimesylate, maleic acid and travoprost has incompatibility under the storage condition of 5° C., and netarsudil dimesylate is precipitated.

### Comparative Examples 49 to 52: A Preparation Including Netarsudil Dimesylate, Timolol Maleate and Tafluprost

TABLE 245
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 49 | | example 50 | | example 51 | | example 52 | |
| 1. | Netarsudil dimesylate | 14.2 | mg | 14.2 | mg | 28.5 | mg | 28.5 | mg |
| 2. | Timolol maleate | 341.7 | mg | 683.5 | mg | 341.7 | mg | 683.5 | mg |
| 3. | Tafluprost | 1.5 | mg | 1.5 | mg | 1.5 | mg | 1.5 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 246
Contents of various main medicine components in Comparative
examples 49 to 52 after being stored at 5° C. for 7 days

| | | Comparative | Comparative | Comparative | Comparative |
|---|---|---|---|---|---|
| | | example 49 | example 50 | example 51 | example 52 |
| No. | Component | Content % | | | |
| 1. | Netarsudil dimesylate | <30 | <30 | <30 | <30 |
| 2. | Timolol maleate | 99.2 | 99.7 | 99.1 | 99.6 |
| 3. | Tafluprost | 100.4 | 100.2 | 99.9 | 100.1 |

According to Comparative examples 49 to 52, the preparation including netarsudil dimesylate, timolol maleate and tafluprost has incompatibility under the storage condition of 5° C., and netarsudil dimesylate is precipitated.

### Comparative Examples 53 to 56: A Preparation Including Netarsudil Dimesylate, Maleic Acid and Tafluprost

TABLE 247
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 53 | | example 54 | | example 55 | | example 56 | |
| 1. | Netarsudil dimesylate | 14.2 | mg | 14.2 | mg | 28.5 | mg | 28.5 | mg |
| 2. | Maleic acid | 91.8 | mg | 183.5 | mg | 91.8 | mg | 183.5 | mg |
| 3. | Tafluprost | 1.5 | mg | 1.5 | mg | 1.5 | mg | 1.5 | mg |
| 4. | Boric acid | 50 | mg | 50 | mg | 50 | mg | 50 | mg |
| 5. | Mannitol | 5000 | mg | 5000 | mg | 5000 | mg | 5000 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 248
Contents of various main medicine components in Comparative
examples 53 to 56 after being stored at 5° C. for 7 days

| | | Comparative | Comparative | Comparative | Comparative |
|---|---|---|---|---|---|
| | | example 53 | example 54 | example 55 | example 56 |
| No. | Component | Content % | | | |
| 1. | Netarsudil dimesylate | <30 | <30 | <30 | <30 |
| 2. | Tafluprost | 99.9 | 99.3 | 99.9 | 100.1 |

According to Comparative examples 53 to 56, the preparation including netarsudil dimesylate, maleic acid and tafluprost has incompatibility under the storage condition of 5° C., and netarsudil dimesylate is precipitated.

### Embodiments 147 to 151: A Preparation Including Netarsudil Dimesylate, Carteolol Mesylate and Latanoprost

TABLE 249
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 147 | | 148 | | 149 | | 150 | | 151 | |
| 1. | Netarsudil dimesylate | 14.2 | mg | 14.2 | mg | 28.5 | mg | 28.5 | mg | 57.0 | mg |
| 2. | Carteolol mesylate | 1329 | mg | 2658 | mg | 1329 | mg | 2658 | mg | 5316 | mg |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 250
Contents of various main medicine components in Embodiments
147 to 151 after being stored at 5° C. for 24 months

| | | Embodiment | Embodiment | Embodiment | Embodiment | Embodiment |
|---|---|---|---|---|---|---|
| | | 147 | 148 | 149 | 150 | 151 |
| No. | Component | Content % | | | | |
| 1. | Netarsudil dimesylate | 100.1 | 99.5 | 101.1 | 100.9 | 99.7 |
| 2. | Carteolol mesylate | 101.3 | 101.5 | 100.8 | 100.8 | 100.9 |
| 3. | Latanoprost | 100.1 | 99.9 | 99.2 | 99.9 | 100.1 |

TABLE 251
Contents of various main medicine components in commercially
available eye drops (Mikelan ®, Rhopress ®, and
Xalatan ®) after being stored at 5° C. for 24 months

| | | Mikelan ® | Rhopress ® | Xalatan ® |
|---|---|---|---|---|
| No. | Component | Content % | | |
| 1. | Carteolol hydrochloride | 99.9 | — | — |
| 2. | Netarsudil dimesylate | — | 99.9 | — |
| 3. | Latanoprost | — | — | 99.8 |

According to Embodiments 147 to 151, the preparation including netarsudil dimesylate, carteolol mesylate and latanoprost has good stability after being stored under a storage condition of 5° C. for 24 months, which meets a requirement for a storage condition of the commercially available eye drops (Mikelan®, Rhopress®, and Xalatan®) before opening.

TABLE 252
Contents of various main medicine components in Embodiments
147 to 151 after being stored at 25° C. for 6 weeks

| | | Embodiment | Embodiment | Embodiment | Embodiment | Embodiment |
|---|---|---|---|---|---|---|
| | | 147 | 148 | 149 | 150 | 151 |
| No. | Component | Content % | | | | |
| 1. | Netarsudil dimesylate | 100.0 | 99.1 | 99.9 | 101.1 | 99.9 |
| 2. | Carteolol mesylate | 100.5 | 101.3 | 99.1 | 101.8 | 100.7 |
| 3. | Latanoprost | 100.2 | 99.8 | 98.7 | 99.9 | 100.0 |

TABLE 253
Contents of various main medicine components in commercially
available eye drops (Mikelan ®, Rhopress ®, and
Xalatan ®) after being stored at 25° C. for 6 weeks

| | | Mikelan ® | Rhopress ® | Xalatan ® |
|---|---|---|---|---|
| No. | Component | Content % | | |
| 1. | Carteolol hydrochloride | 99.9 | — | — |
| 2. | Netarsudil dimesylate | — | 99.8 | — |
| 3. | Latanoprost | — | — | 99.6 |

According to Embodiments 147 to 151, the preparation including netarsudil dimesylate, carteolol mesylate and latanoprost has good stability after being stored under a storage condition of 25° C. for 6 weeks, which meets a requirement for a storage condition of the commercially available eye drops (Mikelan®, Rhopress®, and Xalatan®) after opening.

TABLE 254
Contents of various main medicine components in Embodiments
147 to 151 after being stored at 40° C. for 14 days

| | | Embodiment | Embodiment | Embodiment | Embodiment | Embodiment |
|---|---|---|---|---|---|---|
| | | 147 | 148 | 149 | 150 | 151 |
| No. | Component | Content % | | | | |
| 1. | Netarsudil dimesylate | 99.9 | 98.2 | 98.7 | 99.8 | 99.8 |
| 2. | Carteolol mesylate | 100.0 | 101.9 | 99.9 | 100.4 | 100.9 |
| 3. | Latanoprost | 98.9 | 99.3 | 99.6 | 98.2 | 99.1 |

TABLE 255
Contents of various main medicine components in commercially
available eye drops (Mikelan ®, Rhopress ®, and
Xalatan ®) after being stored at 40° C. for 14 days

| | | Mikelan ® | Rhopress ® | Xalatan ® |
|---|---|---|---|---|
| No. | Component | Content % | | |
| 1. | Carteolol hydrochloride | 101.1 | — | — |
| 2. | Netarsudil dimesylate | — | 99.5 | — |
| 3. | Latanoprost | — | — | 99.7 |

According to Embodiments 147 to 151, the preparation including netarsudil dimesylate, carteolol mesylate and latanoprost has good stability after being stored under a storage condition of 40° C. for 14 days, which meets a requirement for departing from a storage condition of the commercially available eye drops (Mikelan®, Rhopress®, and Xalatan®) for a short time.

### Embodiments 152 to 156: A Preparation Including Netarsudil Dihydrochloride, Carteolol Hydrochloride and Latanoprost

TABLE 256
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 152 | | 153 | | 154 | | 155 | | 156 | |
| 1. | Netarsudil dihydrochloride | 11.6 | mg | 11.6 | mg | 23.2 | mg | 23.2 | mg | 46.4 | mg |
| 2. | Carteolol hydrochloride | 1000 | mg | 2000 | mg | 1000 | mg | 2000 | mg | 4000 | mg |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 257
Contents of various main medicine components in Embodiments
152 to 156 after being stored at 5° C. for 24 months

| | | Embodiment | Embodiment | Embodiment | Embodiment | Embodiment |
|---|---|---|---|---|---|---|
| | | 152 | 153 | 154 | 155 | 156 |
| No. | Component | Content % | | | | |
| 1. | Netarsudil dihydrochloride | 98.5 | 99.3 | 98.8 | 99.0 | 99.8 |
| 2. | Carteolol hydrochloride | 99.7 | 100.0 | 100.1 | 99.9 | 99.8 |
| 3. | Latanoprost | 99.3 | 100.5 | 99.1 | 100.9 | 100.3 |

According to Embodiments 152 to 156, the preparation including netarsudil dihydrochloride, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening.

TABLE 258
Contents of various main medicine components in Embodiments
152 to 156 after being stored at 25° C. for 6 weeks

| | | Embodiment | Embodiment | Embodiment | Embodiment | Embodiment |
|---|---|---|---|---|---|---|
| | | 70 | 71 | 72 | 73 | 74 |
| No. | Component | Content % | | | | |
| 1. | Netarsudil dihydrochloride | 100.1 | 99.9 | 99.4 | 100.3 | 99.8 |
| 2. | Carteolol hydrochloride | 98.6 | 99.5 | 99.9 | 100.4 | 100.2 |
| 3. | Latanoprost | 100.5 | 100.1 | 100.1 | 101.9 | 100.4 |

According to Embodiments 152 to 156, the preparation including netarsudil dihydrochloride, carteolol hydrochloride and latanoprost has good stability after being stored under a storage condition of 25° C. for 6 weeks, which meets a requirement for a storage condition of the commercially available eye drops (Mikelan®, Rhopress®, and Xalatan®) before opening.

TABLE 259
Contents of various main medicine components in Embodiments
152 to 156 after being stored at 40° C. for 14 days

| | | Embodiment | Embodiment | Embodiment | Embodiment | Embodiment |
|---|---|---|---|---|---|---|
| | | 152 | 153 | 154 | 155 | 156 |
| No. | Component | Content % | | | | |
| 1. | Netarsudil dihydrochloride | 100.5 | 99.3 | 99.6 | 100.9 | 100.0 |
| 2. | Carteolol hydrochloride | 99.9 | 99.7 | 101.2 | 101.8 | 99.6 |
| 3. | Latanoprost | 99.9 | 100.2 | 99.9 | 99.7 | 98.2 |

According to Embodiments 152 to 156, the preparation including netarsudil dihydrochloride, carteolol hydrochloride and latanoprost has good stability after being stored under a storage condition of 40° C. for 14 days, which meets a requirement for departing from a storage condition of the commercially available eye drops (Mikelan®, Rhopress®, and Xalatan®) for a short time.

### Embodiment 157: Evaluation of Pharmaceutical Effects of the Ophthalmic Medicine Composition Solutions in Embodiments 147 to 156 by Using a Japanese White Rabbit Glaucoma Model

### Experimental Grouping

Normal control group: no processing.
Model group: an intraocular hypertension model is established, and no eye drop is administered.
Treatment group 5: an intraocular hypertension model is established, and commercially available latanoprost and netarsudil eye drops (Rocklatan®) are administered.
Treatment group 9: an intraocular hypertension model is established, and commercially available carteolol hydrochloride and latanoprost compound eye drops (Mikeluna®) are administered.
Embodiment group 147: an intraocular hypertension model is established, and an ophthalmic medicine composition solution in Embodiment 147 is administered.
Embodiment group 148: an intraocular hypertension model is established, and an ophthalmic medicine composition solution in Embodiment 148 is administered.
Embodiment group 149: an intraocular hypertension model is established, and an ophthalmic medicine composition solution in Embodiment 149 is administered.
Embodiment group 150: an intraocular hypertension model is established, and an ophthalmic medicine composition solution in Embodiment 150 is administered.
Embodiment group 151: an intraocular hypertension model is established, and an ophthalmic medicine composition solution in Embodiment 151 is administered.
Embodiment group 152: an intraocular hypertension model is established, and an ophthalmic medicine composition solution in Embodiment 152 is administered.
Embodiment group 153: an intraocular hypertension model is established, and an ophthalmic medicine composition solution in Embodiment 153 is administered.
Embodiment group 154: an intraocular hypertension model is established, and an ophthalmic medicine composition solution in Embodiment 154 is administered.
Embodiment group 155: an intraocular hypertension model is established, and an ophthalmic medicine composition solution in Embodiment 155 is administered.
Embodiment group 156: an intraocular hypertension model is established, and an ophthalmic medicine composition solution in Embodiment 156 is administered.

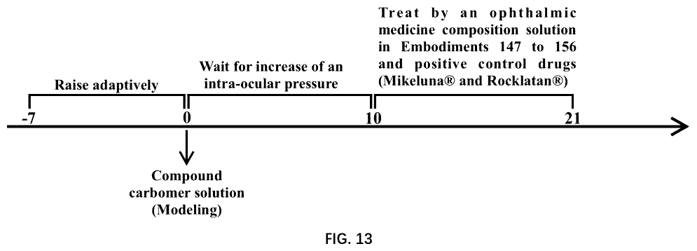

A dosage regimen: according to a current clinical dosage regimen of commercially available latanoprost and netarsudil eye drops (Rocklatan®) and commercially available carteolol hydrochloride and latanoprost compound eye drops (Mikeluna®), this experiment is mainly divided into the following groups: (1) the treatment group 5: 50 μL the commercially available latanoprost and netarsudil eye drops (Rocklatan®) are administered to each eye each time once a day (9:00 PM); (2) the treatment group 9: 50 μL the commercially available carteolol hydrochloride and latanoprost compound eye drops (Mikeluna®) are administered to each eye each time once a day (9:00 PM); and (3) Embodiment groups 147 to 156: 50 μL eye drops in Embodiments 147 to 156 are administered to each eye each time once a day (9:00 PM). Drug administration is started in all the groups on the tenth day when the models are established successfully and lasts for 10 days. Eye drops are not administered to the normal control group and the model group. The dosage regimen is shown in FIG. 13.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 5, the treatment group 9 and Embodiment groups 147, 148, 149, 152, 153 and 154, the IOP can be reduced significantly by the ophthalmic composition solutions of Embodiment groups 150, 151, 155 and 156, however, IOP actual measured values and IOP variation values in Embodiment groups 150, 151, 155 and 156 have no significant difference.

TABLE 260
Difference values between intra-ocular pressure actual measured values of each group of rabbits measured on the tenth
day, the fifteenth day and the twentieth day and an initial intra-ocular pressure (the intra-ocular pressure actual
measured value on the tenth day) when drug administration is performed for 10 days on end starting with the tenth day

| | IOP variation value (mmHg) | | | | | | | | | | | | | |
|---|---|---|---|---|---|---|---|---|---|---|---|---|---|---|
| | | | Treat- | Treat- | Embo- | Embo- | Embo- | Embo- | Embo- | Embo- | Embo- | Embo- | Embo- | Embo- |
| | Normal | | ment | ment | diment | diment | diment | diment | diment | diment | diment | diment | diment | diment |
| Time | control | Model | group | group | group | group | group | group | group | group | group | group | group | group |
| (d) | group | group | 5 | 9 | 147 | 148 | 149 | 150 | 151 | 152 | 153 | 154 | 155 | 156 |
| 10 d | — | — | — | — | — | — | — | — | — | — | — | — | — | — |
| 15 d | 0.50 | 2.25 | 1.50 | −2.48 | −6.12 | −6.56 | −5.50 | −6.22 | −6.80 | −5.70 | −6.40 | −7.40 | −8.40 | −8.10 |
| 20 d | −0.50 | 8.58 | −4.50 | −4.88 | −8.46 | −11.00 | −10.50 | −14.23 | −14.00 | −7.80 | −10.20 | −9.90 | −15.50 | −15.20 |

TABLE 261
Intra-ocular pressure actual measured values of each group of rabbits measured on the tenth day, the fifteenth
day and the twentieth day when drug administration is performed for 10 days on end starting with the tenth day

| | IOP actual measured value (mmHg) | | | | | | | | | | | | | |
|---|---|---|---|---|---|---|---|---|---|---|---|---|---|---|
| | | | Treat- | Treat- | Embo- | Embo- | Embo- | Embo- | Embo- | Embo- | Embo- | Embo- | Embo- | Embo- |
| | Normal | | ment | ment | diment | diment | diment | diment | diment | diment | diment | diment | diment | diment |
| Time | control | Model | group | group | group | group | group | group | group | group | group | group | group | group |
| (d) | group | group | 5 | 9 | 147 | 148 | 149 | 150 | 151 | 152 | 153 | 154 | 155 | 156 |
| 10 d | 6.00 | 26.75 | 24.83 | 24.88 | 25.56 | 26.00 | 25.50 | 24.33 | 24.00 | 25.00 | 25.40 | 25.00 | 25.50 | 25.20 |
| 15 d | 6.50 | 29.00 | 26.33 | 22.40 | 19.44 | 19.44 | 20.00 | 18.11 | 17.20 | 19.30 | 19.00 | 17.60 | 17.10 | 17.10 |
| 20 d | 5.50 | 35.33 | 20.33 | 20.00 | 17.10 | 15.00 | 15.00 | 10.10 | 10.00 | 17.20 | 15.20 | 15.10 | 10.00 | 10.00 |

### Embodiments 158 to 162: A Preparation Including Netarsudil Dimesylate, Carteolol Mesylate and Bimatoprost

TABLE 262
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 158 | | 159 | | 160 | | 161 | | 162 | |
| 1. | Netarsudil dimesylate | 14.2 | mg | 14.2 | mg | 28.5 | mg | 28.5 | mg | 57.0 | mg |
| 2. | Carteolol mesylate | 1329 | mg | 2658 | mg | 1329 | mg | 2658 | mg | 5316 | mg |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 5 | mg | 5 | mg | 5 | mg | 5 | mg | 5 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 263
Contents of various main medicine components in Embodiments
158 to 162 after being stored at 5° C. for 24 months

| | | Embodiment | Embodiment | Embodiment | Embodiment | Embodiment |
|---|---|---|---|---|---|---|
| | | 158 | 159 | 160 | 161 | 162 |
| No. | Component | Content % | | | | |
| 1. | Netarsudil dimesylate | 99.9 | 99.9 | 99.8 | 100.0 | 101.1 |
| 2. | Carteolol mesylate | 100.5 | 100.0 | 99.9 | 100.1 | 100.0 |
| 3. | Bimatoprost | 99.9 | 101.0 | 100.0 | 99.8 | 99.8 |

TABLE 264
Contents of various main medicine components in commercially
available eye drops (Mikelan ®, Rhopress ®, and
Lumigan ®) after being stored at 5° C. for 24 months

| | | Mikelan ® | Rhopress ® | Lumigan ® |
|---|---|---|---|---|
| No. | Component | Content % | | |
| 1. | Carteolol hydrochloride | 99.9 | — | — |
| 2. | Netarsudil dimesylate | — | 99.9 | — |
| 3. | Bimatoprost | — | — | 99.4 |

According to Embodiments 158 to 162, the preparation including netarsudil dimesylate, carteolol mesylate and bimatoprost has good stability after being stored under a storage condition of 5° C. for 24 months, which meets a requirement for a storage condition of the commercially available eye drops (Mikelan®, Rhopress®, and Lumigan®) before opening.

TABLE 265
Contents of various main medicine components in Embodiments
158 to 162 after being stored at 25° C. for 6 weeks

| | | Embodiment | Embodiment | Embodiment | Embodiment | Embodiment |
|---|---|---|---|---|---|---|
| | | 158 | 159 | 160 | 161 | 162 |
| No. | Component | Content % | | | | |
| 1. | Netarsudil dimesylate | 100.0 | 98.1 | 99.6 | 99.3 | 99.9 |
| 2. | Carteolol mesylate | 99.9 | 100.1 | 99.8 | 101.3 | 99.8 |
| 3. | Bimatoprost | 99.5 | 99.3 | 99.9 | 100.0 | 99.5 |

Table 266. Contents of various main medicine components in commercially available eye drops (Mikelan®, Rhopress®, and Lumigan®) after being stored at 25° C. for 6 weeks

TABLE 266
Contents of various main medicine components in commercially
available eye drops (Mikelan ®, Rhopress ®, and
Lumigan ®) after being stored at 25° C. for 6 weeks

| | | Mikelan ® | Rhopress ® | Lumigan ® |
|---|---|---|---|---|
| No. | Component | Content % | | |
| 1. | Carteolol hydrochloride | 99.9 | — | — |
| 2. | Netarsudil dimesylate | — | 99.8 | — |
| 3. | Bimatoprost | — | — | 99.5 |

According to Embodiments 158 to 162, the preparation including netarsudil dimesylate, carteolol mesylate and bimatoprost has good stability after being stored under a storage condition of 25° C. for 6 weeks, which meets a requirement for a storage condition of the commercially available eye drops (Mikelan®, Rhopress®, and Lumigan®) after opening.

TABLE 267
Contents of various main medicine components in Embodiments
158 to 162 after being stored at 40° C. for 14 days

| | | Embodiment | Embodiment | Embodiment | Embodiment | Embodiment |
|---|---|---|---|---|---|---|
| | | 158 | 159 | 160 | 161 | 162 |
| No. | Component | Content % | | | | |
| 1. | Netarsudil dimesylate | 99.8 | 98.5 | 100.0 | 99.1 | 99.4 |
| 2. | Carteolol mesylate | 100.5 | 101.9 | 99.9 | 100.8 | 100.3 |
| 3. | Bimatoprost | 99.2 | 99.6 | 100.6 | 100.5 | 99.8 |

TABLE 268
Contents of various main medicine components in commercially
available eye drops (Mikelan ®, Rhopress ®, and
Lumigan ®) after being stored at 40° C. for 14 days

| | | Mikelan ® | Rhopress ® | Lumigan ® |
|---|---|---|---|---|
| No. | Component | Content % | | |
| 1. | Carteolol hydrochloride | 101.1 | — | — |
| 2. | Netarsudil dimesylate | — | 99.5 | — |
| 3. | Bimatoprost | — | — | 99.2 |

According to Embodiments 158 to 162, the preparation including netarsudil dimesylate, carteolol mesylate and bimatoprost has good stability after being stored under a storage condition of 40° C. for 14 days, which meets a requirement for departing from a storage condition of the commercially available eye drops (Mikelan®, Rhopress®, and Lumigan®) for a short time.

### Embodiments 163 to 167: A Preparation Including Netarsudil Dihydrochloride, Carteolol Hydrochloride and Bimatoprost

TABLE 269
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 163 | | 164 | | 165 | | 166 | | 167 | |
| 1. | Netarsudil dihydrochloride | 11.6 | mg | 11.6 | mg | 23.2 | mg | 23.2 | mg | 46.4 | mg |
| 2. | Carteolol hydrochloride | 1000 | mg | 2000 | mg | 1000 | mg | 2000 | mg | 4000 | mg |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 5 | mg | 5 | mg | 5 | mg | 5 | mg | 5 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 270
Contents of various main medicine components in Embodiments
163 to 167 after being stored at 5° C. for 24 months

| | | Embodiment | Embodiment | Embodiment | Embodiment | Embodiment |
|---|---|---|---|---|---|---|
| | | 163 | 164 | 165 | 166 | 167 |
| No. | Component | Content % | | | | |
| 1. | Netarsudil dihydrochloride | 98.1 | 99.9 | 100.6 | 99.1 | 99.3 |
| 2. | Carteolol hydrochloride | 100.0 | 100.3 | 101.4 | 100.0 | 99.8 |
| 3. | Bimatoprost | 101.8 | 99.4 | 99.8 | 101.3 | 100.5 |

According to Embodiments 163 to 167, the preparation including netarsudil dihydrochloride, carteolol hydrochloride and bimatoprost has good stability after being stored under a storage condition of 5° C. for 24 months, which meets a requirement for a storage condition of the commercially available eye drops (Mikelan®, Rhopress®, and Lumigan®) before opening.

TABLE 271
Contents of various main medicine components in Embodiments
163 to 167 after being stored at 25° C. for 6 weeks

| | | Embodiment | Embodiment | Embodiment | Embodiment | Embodiment |
|---|---|---|---|---|---|---|
| | | 163 | 164 | 165 | 166 | 167 |
| No. | Component | Content % | | | | |
| 1. | Netarsudil dihydrochloride | 99.2 | 100.0 | 99.1 | 101.5 | 99.6 |
| 2. | Carteolol hydrochloride | 100.8 | 99.5 | 100.2 | 99.1 | 100.6 |
| 3. | Bimatoprost | 99.2 | 101.9 | 99.2 | 100.7 | 98.2 |

According to Embodiments 163 to 167, the preparation including netarsudil dihydrochloride, carteolol hydrochloride and bimatoprost has good stability after being stored under a storage condition of 25° C. for 6 weeks, which meets a requirement for a storage condition of the commercially available eye drops (Mikelan®, Rhopress®, and Lumigan®) after opening.

TABLE 272
Contents of various main medicine components in Embodiments
163 to 167 after being stored at 40° C. for 14 days

| | | Embodiment | Embodiment | Embodiment | Embodiment | Embodiment |
|---|---|---|---|---|---|---|
| | | 81 | 82 | 83 | 84 | 85 |
| No. | Component | Content % | | | | |
| 1. | Netarsudil dihydrochloride | 100.7 | 99.3 | 101.8 | 99.2 | 100.2 |
| 2. | Carteolol hydrochloride | 98.7 | 100.5 | 99.7 | 100.0 | 99.1 |
| 3. | Bimatoprost | 100.3 | 98.2 | 101.5 | 99.3 | 100.4 |

According to Embodiments 163 to 167, the preparation including netarsudil dihydrochloride, carteolol hydrochloride and bimatoprost has good stability after being stored under a storage condition of 40° C. for 14 days, which meets a requirement for departing from a storage condition of the commercially available eye drops (Mikelan®, Rhopress®, and Lumigan®) for a short time.

### Embodiment 168: Evaluation of Pharmaceutical Effects of the Ophthalmic Medicine Composition Solutions in Embodiments 158 to 167 by Using a Japanese White Rabbit Glaucoma Model

### Experimental Grouping

Normal control group: no processing.
Model group: an intraocular hypertension model is established, and no eye drop is administered.
Treatment group 5: an intraocular hypertension model is established, and commercially available latanoprost and netarsudil eye drops (Rocklatan®) are administered.
Treatment group 6: an intraocular hypertension model is established, and commercially available bimatoprost and timolol maleate eye drops (Ganfort®) are administered.
Embodiment group 158: an intraocular hypertension model is established, and an ophthalmic medicine composition solution in Embodiment 158 is administered.
Embodiment group 159: an intraocular hypertension model is established, and an ophthalmic medicine composition solution in Embodiment 159 is administered.
Embodiment group 160: an intraocular hypertension model is established, and an ophthalmic medicine composition solution in Embodiment 160 is administered.
Embodiment group 161: an intraocular hypertension model is established, and an ophthalmic medicine composition solution in Embodiment 161 is administered.
Embodiment group 162: an intraocular hypertension model is established, and an ophthalmic medicine composition solution in Embodiment 162 is administered.
Embodiment group 163: an intraocular hypertension model is established, and an ophthalmic medicine composition solution in Embodiment 163 is administered.
Embodiment group 164: an intraocular hypertension model is established, and an ophthalmic medicine composition solution in Embodiment 164 is administered.
Embodiment group 165: an intraocular hypertension model is established, and an ophthalmic medicine composition solution in Embodiment 165 is administered.
Embodiment group 166: an intraocular hypertension model is established, and an ophthalmic medicine composition solution in Embodiment 166 is administered.
Embodiment group 167: an intraocular hypertension model is established, and an ophthalmic medicine composition solution in Embodiment 167 is administered.

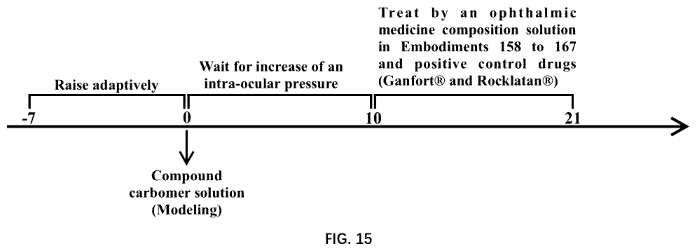

A dosage regimen: according to a current clinical dosage regimen of commercially available latanoprost and netarsudil eye drops (Rocklatan®) and commercially available bimatoprost and timolol maleate eye drops (Ganfort®), this experiment is mainly divided into the following groups: (1) the treatment group 5: 50 μL the commercially available latanoprost and netarsudil eye drops (Rocklatan®) are administered to each eye each time once a day (9:00 PM); (2) the treatment group 6: 50 μL he commercially available bimatoprost and timolol maleate eye drops (Ganfort®) are administered to each eye each time once a day (9:00 PM); and (3) Embodiment groups 158 to 167: 50 μL eye drops in Embodiments 158 to 167 are administered to each eye each time once a day (9:00 PM). Drug administration is started in all the groups on the tenth day when the models are established successfully and lasts for 10 days. Eye drops are not administered to the normal control group and the model group. The dosage regimen is shown in FIG. 15.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 5, the treatment group 6 and Embodiment groups 158, 159, 160, 163, 164 and 165, the IOP can be reduced significantly by the ophthalmic composition solutions of Embodiment groups 161, 162, 166 and 167, however, IOP actual measured values and IOP variation values in Embodiment groups 161, 162, 166 and 167 have no significant difference.

TABLE 273
Difference values between intra-ocular pressure actual measured values of each group of rabbits measured on the tenth
day, the fifteenth day and the twentieth day and an initial intra-ocular pressure (the intra-ocular pressure actual
measured value on the tenth day) when drug administration is performed for 10 days on end starting with the tenth day

| | IOP variation value (mmHg) | | | | | | | | | | | | | |
|---|---|---|---|---|---|---|---|---|---|---|---|---|---|---|
| | | | Treat- | Treat- | Embo- | Embo- | Embo- | Embo- | Embo- | Embo- | Embo- | Embo- | Embo- | Embo- |
| | Normal | | ment | ment | diment | diment | diment | diment | diment | diment | diment | diment | diment | diment |
| Time | control | Model | group | group | group | group | group | group | group | group | group | group | group | group |
| (d) | group | group | 5 | 6 | 158 | 159 | 160 | 161 | 162 | 163 | 164 | 165 | 166 | 167 |
| 10 d | — | — | — | — | — | — | — | — | — | — | — | — | — | — |
| 15 d | 0.50 | 2.25 | 1.50 | −0.83 | −2.44 | −6.70 | −7.39 | −7.90 | −7.30 | −3.38 | −5.45 | −4.47 | −7.25 | −7.70 |
| 20 d | −0.50 | 8.58 | −4.50 | −4.50 | −7.07 | −9.30 | −10.40 | −14.77 | −14.19 | −8.38 | −10.35 | −9.50 | −14.57 | −15.00 |

TABLE 274
Intra-ocular pressure actual measured values of each group of rabbits measured on the tenth day, the fifteenth
day and the twentieth day when drug administration is performed for 10 days on end starting with the tenth day

| | IOP actual measured value (mmHg) | | | | | | | | | | | | | |
|---|---|---|---|---|---|---|---|---|---|---|---|---|---|---|
| | | | Treat- | Treat- | Embo- | Embo- | Embo- | Embo- | Embo- | Embo- | Embo- | Embo- | Embo- | Embo- |
| | Normal | | ment | ment | diment | diment | diment | diment | diment | diment | diment | diment | diment | diment |
| Time | control | Model | group | group | group | group | group | group | group | group | group | group | group | group |
| (d) | group | group | 5 | 6 | 158 | 159 | 160 | 161 | 162 | 163 | 164 | 165 | 166 | 167 |
| 10 d | 6.00 | 26.75 | 24.83 | 24.00 | 25.00 | 24.50 | 25.50 | 26.00 | 25.30 | 25.68 | 25.45 | 24.50 | 24.90 | 25.00 |
| 15 d | 6.50 | 29.00 | 26.33 | 23.17 | 22.56 | 17.80 | 18.11 | 18.10 | 18.00 | 22.30 | 20.00 | 20.03 | 17.65 | 17.30 |
| 20 d | 5.50 | 35.33 | 20.33 | 19.50 | 16.93 | 15.20 | 15.10 | 11.23 | 11.11 | 16.30 | 15.10 | 15.00 | 10.33 | 10.00 |

### Embodiments 169 to 173: A Preparation Including Netarsudil Dimesylate, Carteolol Mesylate and Travoprost

TABLE 275
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 169 | | 170 | | 171 | | 172 | | 173 | |
| 1. | Netarsudil dihydrochloride | 14.2 | mg | 14.2 | mg | 28.5 | mg | 28.5 | mg | 57.0 | mg |
| 2. | Carteolol hydrochloride | 1329 | mg | 2658 | mg | 1329 | mg | 2658 | mg | 5316 | mg |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 276
Contents of various main medicine components in Embodiments
169 to 173 after being stored at 5° C. for 24 months

| | | Embodiment | Embodiment | Embodiment | Embodiment | Embodiment |
|---|---|---|---|---|---|---|
| | | 169 | 170 | 171 | 172 | 173 |
| No. | Component | Content % | | | | |
| 1. | Netarsudil dimesylate | 99.4 | 100.1 | 99.1 | 100.3 | 99.9 |
| 2. | Carteolol mesylate | 100.0 | 98.2 | 100.8 | 99.3 | 98.4 |
| 3. | Travoprost | 99.9 | 98.9 | 100.1 | 99.1 | 99.8 |

TABLE 277
Contents of various main medicine components in commercially
available eye drops (Mikelan ®, Rhopress ®, and
Travatan ®) after being stored at 5° C. for 24 months

| | | Mikelan ® | Rhopress ® | Travatan ® |
|---|---|---|---|---|
| No. | Component | Content % | | |
| 1. | Carteolol hydrochloride | 99.9 | — | — |
| 2. | Netarsudil dimesylate | — | 99.9 | — |
| 3. | Travoprost | — | — | 99.8 |

According to Embodiments 169 to 173, the preparation including netarsudil dimesylate, carteolol mesylate and travoprost has good stability after being stored under a storage condition of 5° C. for 24 months, which meets a requirement for a storage condition of the commercially available eye drops (Mikelan®, Rhopress®, and Travatan®) before opening.

TABLE 278
Contents of various main medicine components in Embodiments
169 to 173 after being stored at 25° C. for 6 weeks

| | | Embodiment | Embodiment | Embodiment | Embodiment | Embodiment |
|---|---|---|---|---|---|---|
| | | 169 | 170 | 171 | 172 | 173 |
| No. | Component | Content % | | | | |
| 1. | Netarsudil dimesylate | 100.1 | 99.1 | 100.5 | 100.3 | 99.5 |
| 2. | Carteolol mesylate | 99.2 | 100.0 | 99.3 | 99.9 | 100.1 |
| 3. | Travoprost | 101.5 | 99.9 | 99.8 | 100.2 | 100.3 |

TABLE 279
Contents of various main medicine components in commercially
available eye drops (Mikelan ®, Rhopress ®, and
Travatan ®) after being stored at 25° C. for 6 weeks

| | | Mikelan ® | Rhopress ® | Travatan ® |
|---|---|---|---|---|
| No. | Component | Content % | | |
| 1. | Carteolol hydrochloride | 99.9 | — | — |
| 2. | Netarsudil dimesylate | — | 99.8 | — |
| 3. | Travoprost | — | — | 99.6 |

According to Embodiments 169 to 173, the preparation including netarsudil dimesylate, carteolol mesylate and travoprost has good stability after being stored under a storage condition of 25° C. for 6 weeks, which meets a requirement for a storage condition of the commercially available eye drops (Mikelan®, Rhopress®, and Travatan®) after opening.

TABLE 280
Contents of various main medicine components in Embodiments
169 to 173 after being stored at 40° C. for 14 days

| | | Embodiment | Embodiment | Embodiment | Embodiment | Embodiment |
|---|---|---|---|---|---|---|
| | | 169 | 170 | 171 | 172 | 173 |
| No. | Component | Content % | | | | |
| 1. | Netarsudil dimesylate | 99.6 | 98.9 | 100.2 | 98.3 | 99.1 |
| 2. | Carteolol mesylate | 99.8 | 100.3 | 99.7 | 99.8 | 100.0 |
| 3. | Travoprost | 100.7 | 99.4 | 99.8 | 100.1 | 99.5 |

TABLE 281
Contents of various main medicine components in commercially
available eye drops (Mikelan ®, Rhopress ®, and
Travatan ®) after being stored at 40° C. for 14 days

| | | Mikelan ® | Rhopress ® | Travatan ® |
|---|---|---|---|---|
| No. | Component | Content % | | |
| 1. | Carteolol hydrochloride | 101.1 | — | — |
| 2. | Netarsudil dimesylate | — | 99.5 | — |
| 3. | Travoprost | — | — | 99.7 |

According to Embodiments 169 to 173, the preparation including netarsudil dimesylate, carteolol mesylate and travoprost has good stability after being stored under a storage condition of 40° C. for 14 days, which meets a requirement for departing from a storage condition of the commercially available eye drops (Mikelan®, Rhopress®, and Travatan®) for a short time.

### Embodiments 174 to 178: A Preparation Including Netarsudil Dihydrochloride, Carteolol Hydrochloride and Travoprost

TABLE 282
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 174 | | 175 | | 176 | | 177 | | 178 | |
| 1. | Netarsudil dihydrochloride | 11.6 | mg | 11.6 | mg | 23.2 | mg | 23.2 | mg | 46.4 | mg |
| 2. | Carteolol hydrochloride | 1000 | mg | 2000 | mg | 1000 | mg | 2000 | mg | 4000 | mg |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 283
Contents of various main medicine components in Embodiments
174 to 178 after being stored at 5° C. for 24 months

| | | Embodiment | Embodiment | Embodiment | Embodiment | Embodiment |
|---|---|---|---|---|---|---|
| | | 174 | 175 | 176 | 177 | 178 |
| No. | Component | Content % | | | | |
| 1. | Netarsudil dihydrochloride | 99.8 | 99.9 | 100.3 | 98.60 | 99.10 |
| 2. | Carteolol hydrochloride | 100.1 | 99.9 | 101.1 | 98.9 | 101.2 |
| 3. | Travoprost | 99.9 | 100.8 | 99.3 | 100.2 | 99.7 |

According to Embodiments 174 to 178, the preparation including netarsudil dihydrochloride, carteolol hydrochloride and travoprost has good stability after being stored under a storage condition of 5° C. for 24 months, which meets a requirement for a storage condition of the commercially available eye drops (Mikelan®, Rhopress®, and Travatan®) before opening.

TABLE 284
Contents of various main medicine components in Embodiments
174 to 178 after being stored at 25° C. for 6 weeks

| | | Embodiment | Embodiment | Embodiment | Embodiment | Embodiment |
|---|---|---|---|---|---|---|
| | | 174 | 175 | 176 | 177 | 178 |
| No. | Component | Content % | | | | |
| 1. | Netarsudil dihydrochloride | 100.5 | 99.1 | 100.3 | 99.5 | 99.9 |
| 2. | Carteolol hydrochloride | 99.9 | 98.9 | 100.5 | 100.1 | 100.2 |
| 3. | Travoprost | 99.3 | 99.8 | 99.7 | 99.8 | 99.9 |

According to Embodiments 174 to 178, the preparation including netarsudil dihydrochloride, carteolol hydrochloride and travoprost has good stability after being stored under a storage condition of 25° C. for 6 weeks, which meets a requirement for a storage condition of the commercially available eye drops (Mikelan®, Rhopress®, and Travatan®) after opening.

TABLE 285
Contents of various main medicine components in Embodiments
174 to 178 after being stored at 40° C. for 14 days

| | | Embodiment | Embodiment | Embodiment | Embodiment | Embodiment |
|---|---|---|---|---|---|---|
| | | 174 | 175 | 176 | 177 | 178 |
| No. | Component | Content % | | | | |
| 1. | Netarsudil dihydrochloride | 99.8 | 99.9 | 101.2 | 100.8 | 100.2 |
| 2. | Carteolol hydrochloride | 100.2 | 100.4 | 100.2 | 99.9 | 99.3 |
| 3. | Travoprost | 99.9 | 99.2 | 100.3 | 99.5 | 100.1 |

According to Embodiments 174 to 178, the preparation including netarsudil dihydrochloride, carteolol hydrochloride and travoprost has good stability after being stored under a storage condition of 40° C. for 14 days, which meets a requirement for departing from a storage condition of the commercially available eye drops (Mikelan®, Rhopress®, and Travatan®) for a short time.

### Embodiment 179: Evaluation of Pharmaceutical Effects of the Ophthalmic Medicine Composition Solutions in Embodiments 169 to 178 by Using a Japanese White Rabbit Glaucoma Model

### Experimental Grouping

Normal control group: no processing.
Model group: an intraocular hypertension model is established, and no eye drop is administered.
Treatment group 5: an intraocular hypertension model is established, and commercially available latanoprost and netarsudil eye drops (Rocklatan®) are administered.
Treatment group 7: an intraocular hypertension model is established, and commercially available travoprost and timolol maleate eye drops (DuoTrav®) are administered.
Embodiment group 169: an intraocular hypertension model is established, and an ophthalmic medicine composition solution in Embodiment 169 is administered.
Embodiment group 170: an intraocular hypertension model is established, and an ophthalmic medicine composition solution in Embodiment 170 is administered.
Embodiment group 171: an intraocular hypertension model is established, and an ophthalmic medicine composition solution in Embodiment 171 is administered.
Embodiment group 172: an intraocular hypertension model is established, and an ophthalmic medicine composition solution in Embodiment 172 is administered.
Embodiment group 173: an intraocular hypertension model is established, and an ophthalmic medicine composition solution in Embodiment 173 is administered.
Embodiment group 174: an intraocular hypertension model is established, and an ophthalmic medicine composition solution in Embodiment 174 is administered.
Embodiment group 175: an intraocular hypertension model is established, and an ophthalmic medicine composition solution in Embodiment 175 is administered.
Embodiment group 176: an intraocular hypertension model is established, and an ophthalmic medicine composition solution in Embodiment 176 is administered.
Embodiment group 177: an intraocular hypertension model is established, and an ophthalmic medicine composition solution in Embodiment 177 is administered.
Embodiment group 178: an intraocular hypertension model is established, and an ophthalmic medicine composition solution in Embodiment 178 is administered.

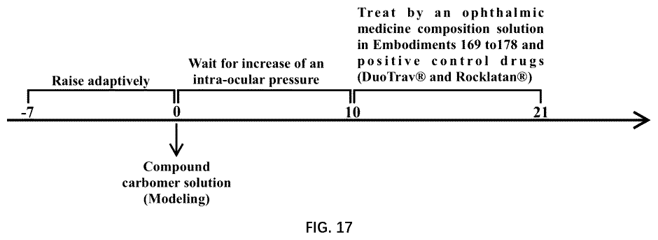

A dosage regimen: according to a current clinical dosage regimen of commercially available latanoprost and netarsudil eye drops (Rocklatan®) and commercially available travoprost and timolol maleate eye drops (DuoTrav®), this experiment is mainly divided into the following groups: (1) the treatment group 5: 50 μL the commercially available latanoprost and netarsudil eye drops (Rocklatan®) are administered to each eye each time once a day (9:00 PM); (2) the treatment group 7: 50 μL the commercially available travoprost and timolol maleate eye drops (DuoTray®) are administered to each eye each time once a day (9:00 PM); and (3) Embodiment groups 169 to 178: 50 μL eye drops in Embodiments 169 to 178 are administered to each eye each time once a day (9:00 PM). Drug administration is started in all the groups on the tenth day when the models are established successfully and lasts for 10 days. Eye drops are not administered to the normal control group and the model group. The dosage regimen is shown in FIG. 17.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 5, the treatment group 7 and Embodiment groups 169, 170, 171, 174, 175 and 176, the IOP can be reduced significantly by the ophthalmic composition solutions of Embodiment groups 172, 173, 177 and 178, however, IOP actual measured values and IOP variation values in Embodiment groups 172, 173, 177 and 178 have no significant difference.

TABLE 286
Difference values between intra-ocular pressure actual measured values of each group of rabbits measured on the tenth
day, the fifteenth day and the twentieth day and an initial intra-ocular pressure (the intra-ocular pressure actual
measured value on the tenth day) when drug administration is performed for 10 days on end starting with the tenth day

| | IOP variation value (mmHg) | | | | | | | | | | | | | |
|---|---|---|---|---|---|---|---|---|---|---|---|---|---|---|
| | | | Treat- | Treat- | Embo- | Embo- | Embo- | Embo- | Embo- | Embo- | Embo- | Embo- | Embo- | Embo- |
| | Normal | | ment | ment | diment | diment | diment | diment | diment | diment | diment | diment | diment | diment |
| Time | control | Model | group | group | group | group | group | group | group | group | group | group | group | group |
| (d) | group | group | 5 | 7 | 169 | 170 | 171 | 172 | 173 | 174 | 175 | 176 | 177 | 178 |
| 10 d | — | — | — | — | — | — | — | — | — | — | — | — | — | — |
| 15 d | 0.50 | 2.25 | 1.50 | −4.00 | −3.05 | −4.67 | −5.40 | −8.60 | −9.60 | −3.49 | −6.55 | −6.20 | −8.65 | −9.40 |
| 20 d | −0.50 | 8.58 | −4.50 | −5.70 | −8.20 | −9.50 | −10.30 | −14.70 | −13.90 | −8.70 | −10.39 | −10.80 | −14.95 | −15.38 |

TABLE 287
Intra-ocular pressure actual measured values of each group of rabbits measured
on the tenth day, the fifteenth day and the twentieth day when drug administration
is performed for 10 days on end starting with the tenth day

| | IOP actual measured value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 5 | group 7 | group 169 | group 170 | group 171 | group 172 |
| 10 d | 6.00 | 26.75 | 24.83 | 25.00 | 25.00 | 24.80 | 25.30 | 25.10 |
| 15 d | 6.50 | 29.00 | 26.33 | 21.00 | 21.95 | 20.13 | 19.90 | 16.50 |
| 20 d | 5.50 | 35.33 | 20.33 | 19.30 | 16.80 | 15.30 | 15.00 | 10.40 |

| | IOP actual measured value (mmHg) | | | | | |
|---|---|---|---|---|---|---|
| Time | Embodiment | Embodiment | Embodiment | Embodiment | Embodiment | Embodiment |
| (d) | group 173 | group 174 | group 175 | group 176 | group 177 | group 178 |
| 10 d | 24.80 | 24.60 | 26.50 | 25.70 | 25.15 | 25.50 |
| 15 d | 15.20 | 21.11 | 19.95 | 19.50 | 16.50 | 16.10 |
| 20 d | 10.90 | 15.90 | 16.11 | 14.90 | 10.20 | 10.12 |

### Embodiments 180 to 184: A Preparation Including Netarsudil Dimesylate, Carteolol Mesylate and Tafluprost

TABLE 288
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 180 | | 181 | | 182 | | 183 | | 184 | |
| 1. | Netarsudil dimesylate | 14.2 | mg | 14.2 | mg | 28.5 | mg | 28.5 | mg | 57.0 | mg |
| 2. | Carteolol mesylate | 1329 | mg | 2658 | mg | 1329 | mg | 2658 | mg | 5316 | mg |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection added by | 100 | mL | 100 | mL | 100 | mL | 100 | mL | 100 | mL |

A preparation process may refer to the preparation process in Embodiments 83 to 87.

TABLE 289
Contents of various main medicine components in Embodiments
180 to 184 after being stored at 5° C. for 24 months

| | | Embodiment | Embodiment | Embodiment | Embodiment | Embodiment |
|---|---|---|---|---|---|---|
| | | 180 | 181 | 182 | 183 | 184 |
| No. | Component | Content % | | | | |
| 1. | Netarsudil dimesylate | 100.0 | 99.1 | 100.4 | 99.8 | 100.3 |
| 2. | Carteolol mesylate | 99.2 | 99.8 | 99.9 | 99.3 | 99.5 |
| 3. | Tafluprost | 99.9 | 100.3 | 99.1 | 100.6 | 100.2 |

TABLE 290
Contents of various main medicine components in commercially
available eye drops (Mikelan ®, Rhopress ®, and
Tapros ®) after being stored at 5° C. for 24 months

| | | Mikelan ® | Rhopress ® | Tapros ® |
|---|---|---|---|---|
| No. | Component | Content % | | |
| 1. | Carteolol hydrochloride | 99.9 | — | — |
| 2. | Netarsudil dimesylate | — | 99.9 | — |
| 3. | Tafluprost | — | — | 99.4 |

According to Embodiments 180 to 184, the preparation including netarsudil dimesylate, carteolol mesylate and tafluprost has good stability after being stored under a storage condition of 5° C. for 24 months, which meets a requirement for a storage condition of the commercially available eye drops (Mikelan®, Rhopress®, and Tapros®) before opening.

TABLE 291
Contents of various main medicine components in Embodiments
180 to 184 after being stored at 25° C. for 6 weeks

| | | Embodiment | Embodiment | Embodiment | Embodiment | Embodiment |
|---|---|---|---|---|---|---|
| | | 180 | 181 | 182 | 183 | 184 |
| No. | Component | Content % | | | | |
| 1. | Netarsudil dimesylate | 99.3 | 100.5 | 101.8 | 99.1 | 99.8 |
| 2. | Carteolol mesylate | 98.1 | 99.6 | 99.9 | 99.3 | 100.2 |
| 3. | Tafluprost | 99.1 | 99.3 | 99.5 | 99.9 | 100.4 |

TABLE 292
Contents of various main medicine components in commercially
available eye drops (Mikelan ®, Rhopress ®, and
Tapros ®) after being stored at 25° C. for 6 weeks

| | | Mikelan ® | Rhopress ® | Tapros ® |
|---|---|---|---|---|
| No. | Component | Content % | | |
| 1. | Carteolol hydrochloride | 99.9 | — | — |
| 2. | Netarsudil dimesylate | — | 99.8 | — |
| 3. | Tafluprost | — | — | 99.3 |

According to Embodiments 180 to 184, the preparation including netarsudil dimesylate, carteolol mesylate and tafluprost has good stability after being stored under a storage condition of 25° C. for 6 weeks, which meets a requirement for a storage condition of the commercially available eye drops (Mikelan®, Rhopress®, and Tapros®) after opening.

TABLE 293
Contents of various main medicine components in Embodiments
180 to 184 after being stored at 40° C. for 14 days

| | | Embodiment | Embodiment | Embodiment | Embodiment | Embodiment |
|---|---|---|---|---|---|---|
| | | 180 | 181 | 182 | 183 | 184 |
| No. | Component | Content % | | | | |
| 1. | Netarsudil dimesylate | 99.5 | 100.2 | 99.9 | 100.7 | 100.2 |
| 2. | Carteolol mesylate | 99.1 | 100.3 | 99.2 | 101.3 | 100.0 |
| 3. | Tafluprost | 99.9 | 100.2 | 99.1 | 99.2 | 100.6 |

TABLE 294
Contents of various main medicine components in commercially
available eye drops (Mikelan ®, Rhopress ®, and
Tapros ®) after being stored at 40° C. for 14 days

| | | Mikelan ® | Rhopress ® | Tapros ® |
|---|---|---|---|---|
| No. | Component | Content % | | |
| 1. | Carteolol hydrochloride | 101.1 | — | — |
| 2. | Netarsudil dimesylate | — | 99.5 | — |
| 3. | Tafluprost | — | — | 99.8 |

According to Embodiments 180 to 184, the preparation including netarsudil dimesylate, carteolol mesylate and tafluprost has good stability after being stored under a storage condition of 40° C. for 14 days, which meets a requirement for departing from a storage condition of the commercially available eye drops (Mikelan®, Rhopress®, and Tapros®) for a short time.

### Embodiments 185 to 189: A Preparation Including Netarsudil Dihydrochloride, Carteolol Hydrochloride and Tafluprost

TABLE 295
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 185 | | 186 | | 187 | | 188 | | 189 | |
| 1. | Netarsudil dihydrochloride | 11.6 | mg | 11.6 | mg | 23.2 | mg | 23.2 | mg | 46.4 | mg |
| 2. | Carteolol hydrochloride | 1000 | mg | 2000 | mg | 1000 | mg | 2000 | mg | 4000 | mg |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 3400 | mg | 3400 | mg | 3400 | mg | 3400 | mg | 3400 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection added by | 100 | mL | 100 | mL | 100 | mL | 100 | mL | 100 | mL |

A preparation process may refer to the preparation process in Embodiments 83 to 87.

TABLE 296
Contents of various main medicine components in Embodiments
185 to 189 after being stored at 5° C. for 24 months

| | | Embodiment | Embodiment | Embodiment | Embodiment | Embodiment |
|---|---|---|---|---|---|---|
| | | 185 | 186 | 187 | 188 | 189 |
| No. | Component | Content % | | | | |
| 1. | Netarsudil dihydrochloride | 99.9 | 99.3 | 100.3 | 99.8 | 99.5 |
| 2. | Carteolol hydrochloride | 100.5 | 100.3 | 99.6 | 99.4 | 101.2 |
| 3. | Tafluprost | 99.7 | 101.6 | 100.9 | 100.3 | 101.9 |

According to Embodiments 185 to 189, the preparation including netarsudil dihydrochloride, carteolol hydrochloride and tafluprost has good stability after being stored under a storage condition of 5° C. for 24 months, which meets a requirement for a storage condition of the commercially available eye drops (Mikelan®, Rhopress®, and Tapro®) before opening.

TABLE 297
Contents of various main medicine components in Embodiments
185 to 189 after being stored at 25° C. for 6 weeks

| | | Embodiment | Embodiment | Embodiment | Embodiment | Embodiment |
|---|---|---|---|---|---|---|
| | | 185 | 186 | 187 | 188 | 189 |
| No. | Component | Content % | | | | |
| 1. | Netarsudil dihydrochloride | 99.3 | 101.1 | 100.4 | 99.4 | 100.6 |
| 2. | Carteolol hydrochloride | 100.7 | 99.9 | 100.2 | 99.1 | 100.4 |
| 3. | Tafluprost | 99.8 | 100.0 | 99.9 | 98.7 | 99.5 |

According to Embodiments 185 to 189, the preparation including netarsudil dihydrochloride, carteolol hydrochloride and tafluprost has good stability after being stored under a storage condition of 25° C. for 6 weeks, which meets a requirement for a storage condition of the commercially available eye drops (Mikelan®, Rhopress®, and Tapros®) after opening.

TABLE 298
Contents of various main medicine components in Embodiments
185 to 189 after being stored at 40° C. for 14 days

| | | Embodiment | Embodiment | Embodiment | Embodiment | Embodiment |
|---|---|---|---|---|---|---|
| | | 185 | 186 | 187 | 188 | 189 |
| No. | Component | Content % | | | | |
| 1. | Netarsudil dihydrochloride | 99.4 | 100.3 | 98.3 | 99.1 | 100.9 |
| 2. | Carteolol hydrochloride | 99.9 | 100.2 | 99.4 | 99.8 | 100.1 |
| 3. | Tafluprost | 100.4 | 99.8 | 100.2 | 101.2 | 99.9 |

According to Embodiments 185 to 189, the preparation including netarsudil dihydrochloride, carteolol hydrochloride and tafluprost has good stability after being stored under a storage condition of 40° C. for 14 days, which meets a requirement for departing from a storage condition of the commercially available eye drops (Mikelan®, Rhopress®, and Tapros®) for a short time.

### Embodiment 190: Evaluation of Pharmaceutical Effects of the Ophthalmic Medicine Composition Solutions in Embodiments 180 to 189 by Using a Japanese White Rabbit Glaucoma Model

### Experimental Grouping

Normal control group: no processing.
Model group: an intraocular hypertension model is established, and no eye drop is administered.
Treatment group 5: an intraocular hypertension model is established, and commercially available latanoprost and netarsudil eye drops (Rocklatan®) are administered.
Treatment group 8: an intraocular hypertension model is established, and commercially available tafluprost and timolol maleate eye drops (Tapcom®) are administered.
Embodiment group 180: an intraocular hypertension model is established, and an ophthalmic medicine composition solution in Embodiment 180 is administered.
Embodiment group 181: an intraocular hypertension model is established, and an ophthalmic medicine composition solution in Embodiment 181 is administered.
Embodiment group 182: an intraocular hypertension model is established, and an ophthalmic medicine composition solution in Embodiment 182 is administered.
Embodiment group 183: an intraocular hypertension model is established, and an ophthalmic medicine composition solution in Embodiment 183 is administered.
Embodiment group 184: an intraocular hypertension model is established, and an ophthalmic medicine composition solution in Embodiment 184 is administered.
Embodiment group 185: an intraocular hypertension model is established, and an ophthalmic medicine composition solution in Embodiment 185 is administered.
Embodiment group 186: an intraocular hypertension model is established, and an ophthalmic medicine composition solution in Embodiment 186 is administered.
Embodiment group 187: an intraocular hypertension model is established, and an ophthalmic medicine composition solution in Embodiment 187 is administered.
Embodiment group 188: an intraocular hypertension model is established, and an ophthalmic medicine composition solution in Embodiment 188 is administered.
Embodiment group 189: an intraocular hypertension model is established, and an ophthalmic medicine composition solution in Embodiment 189 is administered.

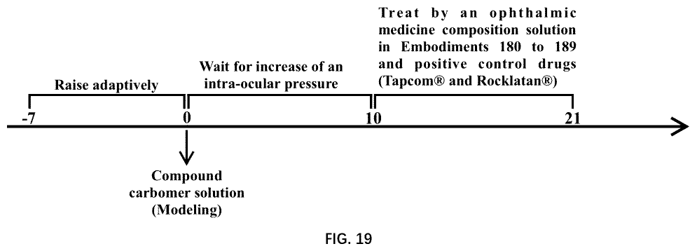

A dosage regimen: according to a current clinical dosage regimen of commercially available latanoprost and netarsudil eye drops (Rocklatan®) and commercially available tafluprost and timolol maleate eye drops (Tapcom®), this experiment is mainly divided into the following groups: (1) the treatment group 5: 50 μL the commercially available latanoprost and netarsudil eye drops (Rocklatan®) are administered to each eye each time once a day (9:00 PM); (2) the treatment group 8: 50 μL the commercially available tafluprost and timolol maleate eye drops (Tapcom®) are administered to each eye each time once a day (9:00 PM); and (3) Embodiment groups 180 to 189: 50 μL eye drops in Embodiments 180 to 189 are administered to each eye each time once a day (9:00 PM). Drug administration is started in all the groups on the tenth day when the models are established successfully and lasts for 10 days. Eye drops are not administered to the normal control group and the model group. Eye drops are not administered to the normal control group and the model group. The dosage regimen is shown in FIG. 19.

The rest of experimental operation procedures (experimental animal, model establishment, and a test index and method) may refer to the description of Embodiment 11.

### Experiment Results

intra-ocular pressure: IOP values in all the treatment groups and the embodiment groups are reduced significantly at all time points after drug administration is performed during a study period compared with the model group (Student's paired t-test). Meanwhile, compared with the treatment group 5, the treatment group 8 and Embodiment groups 180, 181, 182, 185, 186 and 187, the IOP can be reduced significantly by the ophthalmic composition solutions of Embodiment groups 183, 184, 188 and 189, however, IOP actual measured values and IOP variation values in Embodiment groups 183, 184, 188 and 189 have no significant difference.

TABLE 299
Difference values between intra-ocular pressure actual measured values of each group of
rabbits measured on the tenth day, the fifteenth day and the twentieth day and an initial
intra-ocular pressure (the intra-ocular pressure actual measured value on the tenth day)
when drug administration is performed for 10 days on end starting with the tenth day

| | IOP variation value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 5 | group 8 | group 180 | group 181 | group 182 | group 183 |
| 10 d | — | — | — | — | — | — | — | — |
| 15 d | 0.50 | 2.25 | 1.50 | −5.70 | −6.47 | −6.78 | −6.90 | −8.70 |
| 20 d | −0.50 | 8.58 | −4.50 | −6.30 | −8.47 | −9.79 | −10.00 | −14.50 |

| | IOP variation value (mmHg) | | | | | |
|---|---|---|---|---|---|---|
| Time | Embodiment | Embodiment | Embodiment | Embodiment | Embodiment | Embodiment |
| (d) | group 184 | group 185 | group 186 | group 187 | group 188 | group 189 |
| 10 d | — | — | — | — | — | — |
| 15 d | −8.10 | −6.00 | −7.30 | −5.84 | −8.05 | −8.95 |
| 20 d | −14.50 | −8.00 | −10.70 | −10.15 | −15.45 | −14.74 |

TABLE 300
Intra-ocular pressure actual measured values of each group of rabbits measured
on the tenth day, the fifteenth day and the twentieth day when drug administration
is performed for 10 days on end starting with the tenth day

| | IOP actual measured value (mmHg) | | | | | | | |
|---|---|---|---|---|---|---|---|---|
| | Normal | | | | | | | |
| Time | control | Model | Treatment | Treatment | Embodiment | Embodiment | Embodiment | Embodiment |
| (d) | group | group | group 5 | group 8 | group 180 | group 181 | group 182 | group 183 |
| 10 d | 6.00 | 26.75 | 24.83 | 26.00 | 25.87 | 25.10 | 25.00 | 24.80 |
| 15 d | 6.50 | 29.00 | 26.33 | 20.30 | 19.40 | 18.32 | 18.10 | 16.10 |
| 20 d | 5.50 | 35.33 | 20.33 | 19.70 | 17.40 | 15.31 | 15.00 | 10.30 |

| | IOP actual measured value (mmHg) | | | | | |
|---|---|---|---|---|---|---|
| Time | Embodiment | Embodiment | Embodiment | Embodiment | Embodiment | Embodiment |
| (d) | group 184 | group 185 | group 186 | group 187 | group 188 | group 189 |
| 10 d | 24.50 | 25.00 | 25.60 | 25.15 | 25.45 | 24.95 |
| 15 d | 16.40 | 19.00 | 18.30 | 19.31 | 17.40 | 16.00 |
| 20 d | 10.00 | 17.00 | 14.90 | 15.00 | 10.00 | 10.21 |

### Embodiment 192: A Preparation Including Netarsudil Dimesylate, Carteolol Sulfate and Latanoprost

TABLE 301
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol hydrobromide | 2550 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 302
Contents of various main medicine components in
Embodiment 191 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.2 | 100.3 |
| 2. | Carteolol hydrobromide | 100.1 | 99.8 |
| 3. | Latanoprost | 99.7 | 100.5 |

According to Embodiment 191, the preparation including netarsudil dimesylate, carteolol hydrobromide and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 192: A Preparation Including Netarsudil Dimesylate, Carteolol Sulfate and Latanoprost

TABLE 303
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol sulfate | 2680 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 304
Contents of various main medicine components in
Embodiment 192 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.8 | 100.4 |
| 2. | Carteolol sulfate | 100.3 | 99.5 |
| 3. | Latanoprost | 99.9 | 100.1 |

According to Embodiment 192, the preparation including netarsudil dimesylate, carteolol sulfate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 193: A Preparation Including Netarsudil Dimesylate, Carteolol Esilate and Latanoprost

TABLE 305
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol esilate | 3340 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 306
Contents of various main medicine components in
Embodiment 193 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 101.5 | 100.2 |
| 2. | Carteolol esilate | 99.8 | 100.5 |
| 3. | Latanoprost | 100.1 | 99.1 |

According to Embodiment 193, the preparation including netarsudil dimesylate, carteolol esilate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 194: A Preparation Including Netarsudil Dimesylate, Carteolol Nitrate and Latanoprost

TABLE 307
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol nitrate | 2430 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 308
Contents of various main medicine components in
Embodiment 194 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.9 | 99.1 |
| 2. | Carteolol nitrate | 98.2 | 99.0 |
| 3. | Latanoprost | 100.5 | 100.1 |

According to Embodiment 194, the preparation including netarsudil dimesylate, carteolol nitrate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 195: A Preparation Including Netarsudil Dimesylate, Carteolol Citrate and Latanoprost

TABLE 309
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol citrate | 3770 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 310
Contents of various main medicine components in
Embodiment 195 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.7 | 100.1 |
| 2. | Carteolol citrate | 99.9 | 99.8 |
| 3. | Latanoprost | 100.4 | 100.0 |

According to Embodiment 195, the preparation including netarsudil dimesylate, carteolol citrate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 196: A Preparation Including Netarsudil Dimesylate, Carteolol Tartrate and Latanoprost

TABLE 311
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol tartrate | 3030 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s | |
| 8. | Water for injection added by | 100 | mL |

A preparation process may refer to the preparation process in Embodiments 83 to 87.

TABLE 312
Contents of various main medicine components in
Embodiment 196 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.1 | 99.9 |
| 2. | Carteolol tartrate | 99.8 | 99.6 |
| 3. | Latanoprost | 99.1 | 98.2 |

According to Embodiment 196, the preparation including netarsudil dimesylate, carteolol tartrate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 197: A Preparation Including Netarsudil Dimesylate, Carteolol Salicylate and Latanoprost

TABLE 313
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol salicylate | 2940 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

TABLE 314
Contents of various main medicine components in
Embodiment 197 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 98.4 | 99.2 |
| 2. | Carteolol salicylate | 99.7 | 99.3 |
| 3. | Latanoprost | 100.3 | 100.1 |

According to Embodiment 197, the preparation including netarsudil dimesylate, carteolol salicylate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 198: A Preparation Including Netarsudil Dimesylate, Carteolol Malate and Latanoprost

TABLE 315
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol malate | 3690 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in embodiments 83 to 87.

TABLE 316
Contents of various main medicine components in
Embodiment 198 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 101.4 | 100.2 |
| 2. | Carteolol malate | 99.3 | 99.8 |
| 3. | Latanoprost | 100.2 | 100.6 |

According to Embodiment 198, the preparation including netarsudil dimesylate, carteolol malate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 199: A Preparation Including Netarsudil Dimesylate, Carteolol Lactate and Latanoprost

TABLE 317
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol lactate | 2620 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 318
Contents of various main medicine components in
Embodiment 199 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 100.3 | 100.0 |
| 2. | Carteolol lactate | 101.9 | 100.4 |
| 3. | Latanoprost | 99.8 | 99.7 |

According to Embodiment 199, the preparation including netarsudil dimesylate, carteolol lactate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 200: A Preparation Including Netarsudil Dimesylate, Carteolol Phenylacetate and Latanoprost

TABLE 319
Preparation of a local ophthalmic medicine composition
solution for reducing an intra-ocular pressure
through a conventional method as follows:

| No. | Component | Weight | |
|---|---|---|---|
| 1. | Netarsudil dimesylate | 28.5 | mg |
| 2. | Carteolol phenylacetate | 3410 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 320
Contents of various main medicine components in
Embodiment 200 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dimesylate | 99.4 | 99.1 |
| 2. | Carteolol phenylacetate | 99.3 | 99.0 |
| 3. | Latanoprost | 99.6 | 98.6 |

According to Embodiment 200, the preparation including netarsudil dimesylate, carteolol phenylacetate and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 201: A Preparation Including Netarsudil Dihydrobromide, Carteolol Hydrochloride and Latanoprost

TABLE 321
Preparation of a local ophthalmic medicine composition
solution for reducing an intra-ocular pressure
through a conventional method as follows:

| No. | Component | Weight | |
|---|---|---|---|
| 1. | Netarsudil dihydrobromide | 27.1 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 322
Contents of various main medicine components in
Embodiment 201 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dihydrobromide | 100.0 | 101.3 |
| 2. | Carteolol hydrochloride | 99.9 | 98.2 |
| 3. | Latanoprost | 99.2 | 99.6 |

According to Embodiment 201, the preparation including netarsudil dihydrobromide, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 202: A Preparation Including Netarsudil Sulfate, Carteolol Hydrochloride and Latanoprost

TABLE 323
Preparation of a local ophthalmic medicine composition
solution for reducing an intra-ocular pressure
through a conventional method as follows:

| No. | Component | Weight | |
|---|---|---|---|
| 1. | Netarsudil sulfate | 24.3 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 324
Contents of various main medicine components in
Embodiment 202 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil sulfate | 99.4 | 99.6 |
| 2. | Carteolol hydrochloride | 99.2 | 100.2 |
| 3. | Latanoprost | 100.7 | 100.5 |

According to Embodiment 202, the preparation including netarsudil sulfate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 203: a Preparation Including Netarsudil Diformate, Carteolol Hydrochloride and Latanoprost

TABLE 325
Preparation of a local ophthalmic medicine composition
solution for reducing an intra-ocular pressure
through a conventional method as follows:

| No. | Component | Weight | |
|---|---|---|---|
| 1. | Netarsudil diformate | 24.1 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 326
Contents of various main medicine components in
Embodiment 203 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diformate | 99.6 | 99.8 |
| 2. | Carteolol hydrochloride | 99.1 | 99.0 |
| 3. | Latanoprost | 99.9 | 99.7 |

According to Embodiment 203, the preparation including netarsudil diformate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 204: A Preparation Including Netarsudil Dinitrate, Carteolol Hydrochloride and Latanoprost

TABLE 327
Preparation of a local ophthalmic medicine composition
solution for reducing an intra-ocular pressure
through a conventional method as follows:

| No. | Component | Weight | |
|---|---|---|---|
| 1. | Netarsudil dinitrate | 25.6 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 328
Contents of various main medicine components in
Embodiment 204 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dinitrate | 99.3 | 100.5 |
| 2. | Carteolol hydrochloride | 100.7 | 99.9 |
| 3. | Latanoprost | 99.8 | 99.1 |

According to Embodiment 204, the preparation including netarsudil dinitrate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 205: A Preparation Including Netarsudil Diacetate, Carteolol Hydrochloride and Latanoprost

TABLE 329
Preparation of a local ophthalmic medicine composition
solution for reducing an intra-ocular pressure
through a conventional method as follows:

| No. | Component | Weight | |
|---|---|---|---|
| 1. | Netarsudil diacetate | 27.8 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 330
Contents of various main medicine components in
Embodiment 205 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diacetate | 100.7 | 100.2 |
| 2. | Carteolol hydrochloride | 99.3 | 99.6 |
| 3. | Latanoprost | 98.0 | 99.3 |

According to Embodiment 205, the preparation including netarsudil diacetate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 206: A Preparation Including Netarsudil Dibenzoate, Carteolol Hydrochloride and Latanoprost

TABLE 331
Preparation of a local ophthalmic medicine composition
solution for reducing an intra-ocular pressure
through a conventional method as follows:

| No. | Component | Weight | |
|---|---|---|---|
| 1. | Netarsudil dibenzoate | 30.8 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 332
Contents of various main medicine components in
Embodiment 206 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil dibenzoate | 99.4 | 100.2 |
| 2. | Carteolol hydrochloride | 99.6 | 100.0 |
| 3. | Latanoprost | 98.0 | 99.1 |

According to Embodiment 206, the preparation including netarsudil dibenzoate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 207: A Preparation Including Netarsudil Oxalate, Carteolol Hydrochloride and Latanoprost

TABLE 333
Preparation of a local ophthalmic medicine composition
solution for reducing an intra-ocular pressure
through a conventional method as follows:

| No. | Component | Weight | |
|---|---|---|---|
| 1. | Netarsudil oxalate | 27.9 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 334
Contents of various main medicine components in
Embodiment 207 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil oxalate | 100.6 | 100.3 |
| 2. | Carteolol hydrochloride | 99.8 | 99.3 |
| 3. | Latanoprost | 99.5 | 101.3 |

According to Embodiment 207, the preparation including netarsudil oxalate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 208: A Preparation Including Netarsudil Succinate, Carteolol Hydrochloride and Latanoprost

TABLE 335
Preparation of a local ophthalmic medicine composition
solution for reducing an intra-ocular pressure
through a conventional method as follows:

| No. | Component | Weight | |
|---|---|---|---|
| 1. | Netarsudil succinate | 25.2 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 336
Contents of various main medicine components in
Embodiment 208 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil succinate | 99.5 | 99.0 |
| 2. | Carteolol hydrochloride | 100.7 | 100.1 |
| 3. | Latanoprost | 101.3 | 101.5 |

According to Embodiment 208, the preparation including netarsudil succinate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 209: A Preparation Including Netarsudil Diphenylacetate, Carteolol Hydrochloride and Latanoprost

TABLE 337
Preparation of a local ophthalmic medicine composition
solution for reducing an intra-ocular pressure
through a conventional method as follows:

| No. | Component | Weight | |
|---|---|---|---|
| 1. | Netarsudil diphenylacetate | 32.0 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 338
Contents of various main medicine components in
Embodiment 209 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil diphenylacetate | 100.9 | 100.3 |
| 2. | Carteolol hydrochloride | 99.7 | 99.1 |
| 3. | Latanoprost | 99.0 | 98.3 |

According to Embodiment 209, the preparation including netarsudil diphenylacetate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Embodiment 210: A Preparation Including Netarsudil Maleate, Carteolol Hydrochloride and Latanoprost

TABLE 339
Preparation of a local ophthalmic medicine composition
solution for reducing an intra-ocular pressure
through a conventional method as follows:

| No. | Component | Weight | |
|---|---|---|---|
| 1. | Netarsudil maleate | 25.1 | mg |
| 2. | Carteolol hydrochloride | 2000 | mg |
| 3. | Latanoprost | 5 | mg |
| 4. | Boric acid | 50 | mg |
| 5. | Mannitol | 3400 | mg |
| 6. | Benzalkonium chloride | 20 | mg |
| 7. | Sodium hydroxide | q.s. | |
| 8. | Water for injection added by | 100 | mL |

A preparation process may refer to the preparation process in Embodiments 83 to 87.

TABLE 340
Contents of various main medicine components in
Embodiment 210 after being stored at 5° C.
for 24 months and at 25° C. for 6 weeks

| | |
|---|---|
| 5° C. | 25° C. |

| | | |
|---|---|---|
| No. | Component | Content % |

| | | | |
|---|---|---|---|
| 1. | Netarsudil maleate | 100.1 | 100.3 |
| 2. | Carteolol hydrochloride | 99.7 | 99.1 |
| 3. | Latanoprost | 99.3 | 99.6 |

According to Embodiment 210, the preparation including netarsudil maleate, carteolol hydrochloride and latanoprost has good stability after being stored under a storage condition of 5° C. for 24 months, which meets a requirement for a storage condition of the commercially available eye drops (Mikelan®, Rhopress®, and Xalatan®) before opening; and has good stability after being stored under a storage condition of 25° C. for 6 weeks, which meets a requirement for a storage condition of the commercially available eye drops (Mikelan®, Rhopress®, and Xalatan®) after opening.

### Comparative Examples 81 to 84: A Preparation Including Netarsudil Dimesylate and Carteolol Hydrochloride

TABLE 341
Preparation of a local ophthalmic medicine composition solution for reducing
an intra-ocular pressure through a conventional method as follows:

| | | Comparative | | Comparative | | Comparative | | Comparative | |
|---|---|---|---|---|---|---|---|---|---|
| No. | Component | example 57 | | example 58 | | example 59 | | example 60 | |
| 1. | Netarsudil dimesylate | 14.2 | mg | 14.2 | mg | 28.5 | mg | 28.5 | mg |
| 2. | Carteolol hydrochloride | 1000 | mg | 2000 | mg | 1000 | mg | 2000 | mg |
| 3. | Latanoprost | 5 | mg | 5 | mg | 5 | mg | 5 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 342
Contents of various main medicine components in Comparative
examples 81 to 84 after being stored at 5° C. for 7 days

| | | Comparative | Comparative | Comparative | Comparative |
|---|---|---|---|---|---|
| | | example 57 | example 58 | example 59 | example 60 |
| No. | Component | Content % | | | |
| 1. | Netarsudil dimesylate | <30 | <30 | <30 | <30 |
| 2. | Carteolol hydrochloride | 99.9 | 100.4 | 100.5 | 100.7 |
| 3. | Latanoprost | 99.7 | 99.6 | 100.1 | 99.5 |

According to Comparative examples 81 to 84: the preparation including netarsudil dimesylate, carteolol hydrochloride and latanoprost has incompatibility under the storage condition of 5° C., and netarsudil dimesylate is precipitated.

### Comparative Examples 85 to 88: A Preparation Including Netarsudil Dimesylate and Hydrochloric Acid

TABLE 343
Preparation of a local ophthalmic medicine composition solution for reducing
an intra-ocular pressure through a conventional method as follows:

| | | Comparative | | Comparative | | Comparative | | Comparative | |
|---|---|---|---|---|---|---|---|---|---|
| No. | Component | example 61 | | example 62 | | example 63 | | example 64 | |
| 1. | Netarsudil dimesylate | 14.2 | mg | 14.2 | mg | 28.5 | mg | 28.5 | mg |
| 2. | Hydrochloric acid | 0.111 | mL | 0.188 | mL | 0.094 | mL | 0.188 | mL |
| 3. | Latanoprost | 5 | mg | 5 | mg | 5 | mg | 5 | mg |
| 3. | Boric acid | 50 | mg | 50 | mg | 50 | mg | 50 | mg |
| 4. | Mannitol | 3400 | mg | 3400 | mg | 3400 | mg | 3400 | mg |
| 5. | Benzalkonium chloride | 20 | mg | 20 | mg | 20 | mg | 20 | mg |
| 6. | Sodium hydroxide | q.s. | | q.s. | | q.s. | | q.s. | |
| 7. | Water for injection added | 100 | mL | 100 | mL | 100 | mL | 100 | mL |

by

A preparation process may refer to the preparation process in Embodiments 83 to 87.

TABLE 344
Contents of various main medicine components in Comparative
examples 85 to 88 after being stored at 5° C. for 7 days

| | | Comparative | Comparative | Comparative | Comparative |
|---|---|---|---|---|---|
| | | example 61 | example 62 | example 63 | example 64 |
| No. | Component | Content % | | | |
| 1. | Netarsudil dimesylate | <30 | <30 | <30 | <30 |
| 2. | Latanoprost | 99.1 | 98.7 | 99.5 | 99.8 |

According to Comparative examples 85 to 88, the preparation including netarsudil dimesylate, hydrochloric acid and latanoprost has incompatibility under the storage condition of 5° C., and netarsudil dimesylate is precipitated.

### Comparative Examples 65 to 68: A Preparation Including Netarsudil Dimesylate, Carteolol Hydrochloride and Bimatoprost

TABLE 345
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 65 | | example 66 | | example 67 | | example 68 | |
| 1. | Netarsudil dimesylate | 14.2 | mg | 14.2 | mg | 28.5 | mg | 28.5 | mg |
| 2. | Carteolol hydrochloride | 1000 | mg | 2000 | mg | 1000 | mg | 2000 | mg |
| 3. | Bimatoprost | 30 | mg | 30 | mg | 30 | mg | 30 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 346
Contents of various main medicine components in Comparative
examples 65 to 68 after being stored at 5° C. for 7 days

| | | Comparative | Comparative | Comparative | Comparative |
|---|---|---|---|---|---|
| | | example 65 | example 66 | example 67 | example 68 |
| No. | Component | Content % | | | |
| 1. | Netarsudil dimesylate | <30 | <30 | <30 | <30 |
| 2. | Carteolol hydrochloride | 100.1 | 100.3 | 100.0 | 100.2 |
| 3. | Bimatoprost | 99.8 | 101.3 | 100.9 | 99.8 |

According to Comparative examples 65 to 68: the preparation including netarsudil dimesylate, carteolol hydrochloride and bimatoprost has incompatibility under the storage condition of 5° C., and netarsudil dimesylate is precipitated.

### Comparative Examples 69 to 72: A Preparation Including Netarsudil Dimesylate, Hydrochloric Acid and Bimatoprost

TABLE 347
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 69 | | example 70 | | example 71 | | example 72 | |
| 1. | Netarsudil dimesylate | 14.2 | mg | 14.2 | mg | 28.5 | mg | 28.5 | mg |
| 2. | Hydrochloric acid | 0.094 | mL | 0.188 | mL | 0.094 | mL | 0.188 | mL |
| 3. | Bimatoprost | 30 | mg | 30 | mg | 30 | mg | 30 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 348
Contents of various main medicine components in Comparative
examples 69 to 72 after being stored at 5° C. for 7 days

| | | Comparative | Comparative | Comparative | Comparative |
|---|---|---|---|---|---|
| | | example 69 | example 70 | example 71 | example 72 |
| No. | Component | Content % | | | |
| 1. | Netarsudil dimesylate | <30 | <30 | <30 | <30 |
| 2. | Bimatoprost | 99.7 | 99.5 | 99.5 | 99.6 |

According to Comparative examples 69 to 72, the preparation including netarsudil dimesylate, hydrochloric acid and bimatoprost has incompatibility under the storage condition of 5° C., and netarsudil dimesylate is precipitated.

### Comparative Examples 73 to 76: A Preparation Including Netarsudil Dimesylate, Carteolol Hydrochloride and Travoprost

TABLE 349
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 73 | | example 74 | | example 75 | | example 76 | |
| 1. | Netarsudil dimesylate | 14.2 | mg | 14.2 | mg | 28.5 | mg | 28.5 | mg |
| 2. | Carteolol hydrochloride | 1000 | mg | 2000 | mg | 1000 | mg | 2000 | mg |
| 3. | Travoprost | 4 | mg | 4 | mg | 4 | mg | 4 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 350
Contents of various main medicine components in Comparative
examples 73 to 76 after being stored at 5° C. for 7 days

| | | Comparative | Comparative | Comparative | Comparative |
|---|---|---|---|---|---|
| | | example 73 | example 74 | example 75 | example 76 |
| No. | Component | Content % | | | |
| 1. | Netarsudil dimesylate | <30 | <30 | <30 | <30 |
| 2. | Carteolol hydrochloride | 100.2 | 100.5 | 100.8 | 100.1 |
| 3. | Travoprost | 99.8 | 99.7 | 99.5 | 99.1 |

According to Comparative examples 73 to 76, the preparation including netarsudil dimesylate, carteolol hydrochloride and travoprost has incompatibility under the storage condition of 5° C., and netarsudil dimesylate is precipitated.

### Comparative Examples 77 to 80: A Preparation Including Netarsudil Dimesylate, Hydrochloric Acid and Travoprost

TABLE 351
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 77 | | example 78 | | example 79 | | example 80 | |
| 1. | Netarsudil dimesylate | 14.2 | mg | 14.2 | mg | 28.5 | mg | 28.5 | mg |
| 2. | Hydrochloric acid | 0.094 | mL | 0.188 | mL | 0.094 | mL | 0.188 | mL |
| 3. | Travoprost | 4 | mg | 4 | mg | 4 | mg | 4 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 352
Contents of various main medicine components in Comparative
examples 77 to 80 after being stored at 5° C. for 7 days

| | | Comparative | Comparative | Comparative | Comparative |
|---|---|---|---|---|---|
| | | example 77 | example 78 | example 79 | example 80 |
| No. | Component | Content % | | | |
| 1. | Netarsudil dimesylate | <30 | <30 | <30 | <30 |
| 2. | Travoprost | 100.3 | 99.8 | 100.1 | 99.9 |

According to Comparative examples 77 to 80, the preparation including netarsudil dimesylate, hydrochloric acid and travoprost has incompatibility under the storage condition of 5° C., and netarsudil dimesylate is precipitated.

### Comparative Examples 81 to 84: A Preparation Including Netarsudil Dimesylate, Carteolol Hydrochloride and Tafluprost

TABLE 353
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 81 | | example 82 | | example 83 | | example 84 | |
| 1. | Netarsudil dimesylate | 14.2 | mg | 14.2 | mg | 28.5 | mg | 28.5 | mg |
| 2. | Carteolol hydrochloride | 1000 | mg | 2000 | mg | 1000 | mg | 2000 | mg |
| 3. | Tafluprost | 1.5 | mg | 1.5 | mg | 1.5 | mg | 1.5 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 354
Contents of various main medicine components in Comparative
examples 81 to 84 after being stored at 5° C. for 7 days

| | | Comparative | Comparative | Comparative | Comparative |
|---|---|---|---|---|---|
| | | example 81 | example 82 | example 83 | example 84 |
| No. | Component | Content % | | | |
| 1. | Netarsudil dimesylate | <30 | <30 | <30 | <30 |
| 2. | Carteolol hydrochloride | 100.3 | 100.0 | 99.9 | 100.1 |
| 3. | Tafluprost | 99.5 | 99.8 | 99.1 | 99.4 |

According to Comparative examples 81 to 84, the preparation including netarsudil dimesylate, carteolol hydrochloride and tafluprost has incompatibility under the storage condition of 5° C., and netarsudil dimesylate is precipitated.

### Comparative Examples 85 to 88: A Preparation Including Netarsudil Dimesylate, Hydrochloric Acid and Tafluprost

TABLE 355
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 85 | | example 86 | | example 87 | | example 88 | |
| 1. | Netarsudil dimesylate | 14.2 | mg | 14.2 | mg | 28.5 | mg | 28.5 | mg |
| 2. | Hydrochloric acid | 0.094 | mL | 0.188 | mL | 0.094 | mL | 0.188 | mL |
| 3. | Tafluprost | 1.5 | mg | 1.5 | mg | 1.5 | mg | 1.5 | mg |
| 4. | Boric acid | 50 | mg | 50 | mg | 50 | mg | 50 | mg |
| 5. | Mannitol | 3400 | mg | 3400 | mg | 3400 | mg | 3400 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added by | 100 | mL | 100 | mL | 100 | mL | 100 | mL |

A preparation process may refer to the preparation process in Embodiments 83 to 87.

TABLE 356
Contents of various main medicine components in Comparative
examples 85 to 88 after being stored at 5° C. for 7 days

| | | Comparative | Comparative | Comparative | Comparative |
|---|---|---|---|---|---|
| | | example 85 | example 86 | example 87 | example 88 |
| No. | Component | Content % | | | |
| 1. | Netarsudil dimesylate | <30 | <30 | <30 | <30 |
| 2. | Tafluprost | 99.9 | 99.3 | 99.9 | 100.1 |

According to Comparative examples 85 to 88, the preparation including netarsudil dimesylate, hydrochloric acid and tafluprost has incompatibility under the storage condition of 5° C., and netarsudil dimesylate is precipitated.

### Embodiments 211 to 215: A Preparation Including Netarsudil Dimesylate, Betaxolol Mesylate and Latanoprost

TABLE 357
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 211 | | 212 | | 213 | | 214 | | 215 | |
| 1. | Netarsudil dimesylate | 14.2 | mg | 14.2 | mg | 28.5 | mg | 28.5 | mg | 57.0 | mg |
| 2. | Betaxolol mesylate | 164.1 | mg | 328.2 | mg | 164.1 | mg | 328.2 | mg | 656.3 | mg |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection added by | 100 | mL | 100 | mL | 100 | mL | 100 | mL | 100 | mL |

A preparation process may refer to the preparation process in Embodiments 83 to 87.

TABLE 358
Contents of various main medicine components in Embodiments
211 to 215 after being stored at 5° C. for 24 months

| | | Embodiment | Embodiment | Embodiment | Embodiment | Embodiment |
|---|---|---|---|---|---|---|
| | | 211 | 212 | 213 | 214 | 215 |
| No. | Component | Content % | | | | |
| 1. | Netarsudil dimesylate | 100.1 | 99.2 | 100.3 | 99.9 | 99.3 |
| 2. | Betaxolol mesylate | 100.3 | 101.5 | 100.3 | 100.5 | 99.9 |
| 3. | Latanoprost | 100.1 | 99.2 | 99.5 | 99.5 | 100.3 |

TABLE 359
Contents of various main medicine components in commercially
available eye drops (Betoptic ®, Rhopress ®, and
Xalatan ®) after being stored at 5° C. for 24 months

| | | Betoptic ® | Rhopress ® | Xalatan ® |
|---|---|---|---|---|
| No. | Component | Content % | | |
| 1. | Betaxolol hydrochloride | 99.9 | — | — |
| 2. | Netarsudil dimesylate | — | 99.9 | — |
| 3. | Latanoprost | — | — | 99.8 |

According to Embodiments 211 to 215, the preparation including netarsudil dimesylate, betaxolol mesylate and latanoprost has good stability after being stored under a storage condition of 5° C. for 24 months, which meets a requirement for a storage condition of the commercially available eye drops (Betoptic®, Rhopress®, and Xalatan®) before opening.

TABLE 360
Contents of various main medicine components in Embodiments
211 to 215 after being stored at 25° C. for 6 weeks

| | | Embodiment | Embodiment | Embodiment | Embodiment | Embodiment |
|---|---|---|---|---|---|---|
| | | 211 | 212 | 213 | 214 | 215 |
| No. | Component | Content % | | | | |
| 1. | Netarsudil dimesylate | 100.0 | 99.1 | 99.9 | 101.1 | 99.9 |
| 2. | Betaxolol mesylate | 100.5 | 100.3 | 101.1 | 101.8 | 100.7 |
| 3. | Latanoprost | 100.2 | 99.8 | 98.7 | 99.9 | 100.0 |

TABLE 361
Contents of various main medicine components in commercially
available eye drops (Betoptic ®, Rhopress ®, and
Xalatan ®) after being stored at 25° C. for 6 weeks

| | | Betoptic ® | Rhopress ® | Xalatan ® |
|---|---|---|---|---|
| No. | Component | Content % | | |
| 1. | Betaxolol hydrochloride | 99.7 | — | — |
| 2. | Netarsudil dimesylate | — | 99.8 | — |
| 3. | Latanoprost | — | — | 99.6 |

According to Embodiments 211 to 215, the preparation including netarsudil dimesylate, betaxolol mesylate and latanoprost has good stability after being stored under a storage condition of 25° C. for 6 weeks, which meets a requirement for a storage condition of the commercially available eye drops (Betoptic®, Rhopress®, and Xalatan®) after opening.

TABLE 362
Contents of various main medicine components in Embodiments
211 to 215 after being stored at 40° C. for 14 days

| | | Embodiment | Embodiment | Embodiment | Embodiment | Embodiment |
|---|---|---|---|---|---|---|
| | | 211 | 212 | 213 | 214 | 215 |
| No. | Component | Content % | | | | |
| 1. | Netarsudil dimesylate | 99.3 | 99.2 | 99.1 | 98.9 | 99.1 |
| 2. | Betaxolol mesylate | 100.9 | 101.1 | 98.9 | 100.1 | 101.9 |
| 3. | Latanoprost | 99.1 | 99.2 | 98.6 | 98.9 | 99.2 |

TABLE 363
Contents of various main medicine components in commercially
available eye drops (Betoptic ®, Rhopress ®, and
Xalatan ®) after being stored at 40° C. for 14 days

| | | Betoptic ® | Rhopress ® | Xalatan ® |
|---|---|---|---|---|
| No. | Component | Content % | | |
| 1. | Betaxolol hydrochloride | 100.1 | — | — |
| 2. | Netarsudil dimesylate | — | 99.5 | — |
| 3. | Latanoprost | — | — | 99.7 |

According to Embodiments 211 to 215, the preparation including netarsudil dimesylate, betaxolol mesylate and latanoprost has good stability after being stored under a storage condition of 40° C. for 14 days, which meets a requirement for departing from a storage condition of the commercially available eye drops (Betoptic®, Rhopress®, and Xalatan®) for a short time.

### Embodiments 216 to 220: A Preparation Including Netarsudil Dihydrochloride, Betaxolol Hydrochloride and Latanoprost

TABLE 364
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 216 | | 217 | | 218 | | 219 | | 220 | |
| 1. | Netarsudil | 11.6 | mg | 11.6 | mg | 23.2 | mg | 23.2 | mg | 46.4 | mg |
| | dihydrochloride | | | | | | | | | | |
| 2. | Betaxolol | 139.8 | mg | 279.6 | mg | 139.8 | mg | 279.6 | mg | 559.3 | mg |
| | hydrochloride | | | | | | | | | | |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 365
Contents of various main medicine components in Embodiments
216 to 220 after being stored at 5° C. for 24 months

| | | Embodiment | Embodiment | Embodiment | Embodiment | Embodiment |
|---|---|---|---|---|---|---|
| | | 216 | 217 | 218 | 219 | 220 |
| No. | Component | Content % | | | | |
| 1. | Netarsudil | 99.5 | 99.2 | 99.7 | 99.3 | 99.9 |
| | dihydrochloride | | | | | |
| 2. | Betaxolol hydrochloride | 99.3 | 100.1 | 100.9 | 99.7 | 99.9 |
| 3. | Latanoprost | 99.8 | 100.3 | 99.8 | 100.5 | 100.1 |

According to Embodiments 216 to 220, the preparation including netarsudil dihydrochloride, betaxolol hydrochloride and latanoprost has good stability after being stored under a storage condition of 5° C. for 24 months, which meets a requirement for a storage condition of the commercially available eye drops (Betoptic®, Rhopress®, and Xalatan®) before opening.

TABLE 366
Contents of various main medicine components in Embodiments
216 to 220 after being stored at 25° C. for 6 weeks

| | | Embodiment | Embodiment | Embodiment | Embodiment | Embodiment |
|---|---|---|---|---|---|---|
| | | 216 | 217 | 218 | 219 | 220 |
| No. | Component | Content % | | | | |
| 1. | Netarsudil | 100.4 | 99.5 | 99.9 | 100.1 | 99.9 |
| | dihydrochloride | | | | | |
| 2. | Betaxolol hydrochloride | 99.6 | 99.4 | 99.3 | 100.1 | 100.1 |
| 3. | Latanoprost | 101.5 | 101.1 | 100.7 | 100.9 | 100.2 |

According to Embodiments 21 to 220, the preparation including netarsudil dihydrochloride, betaxolol hydrochloride and latanoprost has good stability after being stored under a storage condition of 25° C. for 6 weeks, which meets a requirement for a storage condition of the commercially available eye drops (Betoptic®, Rhopress®, and Xalatan®) before opening.

TABLE 367
Contents of various main medicine components in Embodiments
216 to 220 after being stored at 40° C. for 14 days

| | | Embodiment | Embodiment | Embodiment | Embodiment | Embodiment |
|---|---|---|---|---|---|---|
| | | 216 | 217 | 218 | 219 | 220 |
| No. | Component | Content % | | | | |
| 1. | Netarsudil | 100.1 | 99.1 | 99.8 | 100.1 | 100.8 |
| | dihydrochloride | | | | | |
| 2. | Betaxolol hydrochloride | 99.2 | 99.6 | 101.3 | 100.8 | 99.3 |
| 3. | Latanoprost | 99.4 | 100.3 | 99.4 | 99.8 | 99.2 |

According to Embodiments 216 to 220, the preparation including netarsudil dihydrochloride, betaxolol hydrochloride and latanoprost has good stability after being stored under a storage condition of 40° C. for 14 days, which meets a requirement for departing from a storage condition of the commercially available eye drops (Betoptic®, Rhopress®, and Xalatan®) for a short time.

### Embodiments 221 to 225: A Preparation Including Netarsudil Dimesylate, Betaxolol Mesylate and Bimatoprost

TABLE 368
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 221 | | 222 | | 223 | | 224 | | 225 | |
| 1. | Netarsudil dimesylate | 14.2 | mg | 14.2 | mg | 28.5 | mg | 28.5 | mg | 57.0 | mg |
| 2. | Betaxolol mesylate | 164.1 | mg | 328.2 | mg | 164.1 | mg | 328.2 | mg | 656.3 | mg |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 5000 | mg | 5000 | mg | 5000 | mg | 5000 | mg | 5000 | mg |
| 6. | Benzalkonium chloride | 5 | mg | 5 | mg | 5 | mg | 5 | mg | 5 | mg |
| 7. | Sodium hydroxide | q.s. | | q.s. | | q.s. | | q.s. | | q.s. | |
| 8. | Water for injection added | 100 | mL | 100 | mL | 100 | mL | 100 | mL | 100 | mL |

by

A preparation process may refer to the preparation process in Embodiments 83 to 87.

TABLE 369
Contents of various main medicine components in Embodiments
221 to 225 after being stored at 5° C. for 24 months

| | | Embodiment | Embodiment | Embodiment | Embodiment | Embodiment |
|---|---|---|---|---|---|---|
| | | 221 | 222 | 223 | 224 | 225 |
| No. | Component | Content % | | | | |
| 1. | Netarsudil dimesylate | 99.2 | 99.5 | 100.8 | 100.3 | 100.1 |
| 2. | Betaxolol mesylate | 100.1 | 100.2 | 100.9 | 100.9 | 100.4 |
| 3. | Bimatoprost | 99.6 | 101.2 | 100.4 | 99.3 | 99.9 |

TABLE 370
Contents of various main medicine components in commercially
available eye drops (Betoptic ®, Rhopress ®, and
Lumigan ®) after being stored at 5° C. for 24 months

| | | Betoptic ® | Rhopress ® | Lumigan ® |
|---|---|---|---|---|
| No. | Component | Content % | | |
| 1. | Betaxolol hydrochloride | 99.9 | — | — |
| 2. | Netarsudil dimesylate | — | 99.9 | — |
| 3. | Bimatoprost | — | — | 99.4 |

According to Embodiments 221 to 225, the preparation including netarsudil dimesylate, betaxolol mesylate and bimatoprost has good stability after being stored under a storage condition of 5° C. for 24 months, which meets a requirement for a storage condition of the commercially available eye drops (Betoptic®, Rhopress®, and Lumigan®) before opening.

TABLE 371
Contents of various main medicine components in Embodiments
221 to 225 after being stored at 25° C. for 6 weeks

| | | Embodiment | Embodiment | Embodiment | Embodiment | Embodiment |
|---|---|---|---|---|---|---|
| | | 221 | 222 | 223 | 224 | 225 |
| No. | Component | Content % | | | | |
| 1. | Netarsudil dimesylate | 100.1 | 99.1 | 99.7 | 99.4 | 99.0 |
| 2. | Betaxolol mesylate | 99.5 | 101.1 | 99.5 | 100.3 | 99.3 |
| 3. | Bimatoprost | 99.9 | 99.2 | 100.9 | 100.3 | 100.5 |

TABLE 372
Contents of various main medicine components in commercially
available eye drops (Betoptic ®, Rhopress ®, and
Lumigan ®) after being stored at 25° C. for 6 weeks

| | | Betoptic ® | Rhopress ® | Lumigan |
|---|---|---|---|---|
| No. | Component | Content % | | |
| 1. | Betaxolol hydrochloride | 99.7 | — | — |
| 2. | Netarsudil dimesylate | — | 99.8 | — |
| 3. | Bimatoprost | — | — | 99.5 |

According to Embodiments 221 to, the preparation including netarsudil dimesylate, betaxolol mesylate and bimatoprost has good stability after being stored under a storage condition of 25° C. for 6 weeks, which meets a requirement for a storage condition of the commercially available eye drops (Betoptic®, Rhopress®, and Lumigan®) after opening.

TABLE 373
Contents of various main medicine components in Embodiments
221 to 225 after being stored at 40° C. for 14 days

| | | Embodiment | Embodiment | Embodiment | Embodiment | Embodiment |
|---|---|---|---|---|---|---|
| | | 221 | 222 | 223 | 224 | 225 |
| No. | Component | Content % | | | | |
| 1. | Netarsudil dimesylate | 99.5 | 99.5 | 100.1 | 99.5 | 99.8 |
| 2. | Betaxolol mesylate | 100.1 | 100.9 | 99.7 | 101.8 | 100.1 |
| 3. | Bimatoprost | 99.7 | 99.9 | 100.1 | 98.5 | 99.5 |

TABLE 374
Contents of various main medicine components in commercially
available eye drops (Betoptic ®, Rhopress ®, and
Lumigan ®) after being stored at 40° C. for 14 days

| | | Betoptic ® | Rhopress ® | Lumigan ® |
|---|---|---|---|---|
| No. | Component | Content % | | |
| 1. | Betaxolol hydrochloride | 100.1 | — | — |
| 2. | Netarsudil dimesylate | — | 99.5 | — |
| 3. | Bimatoprost | — | — | 99.2 |

According to Embodiments 221 to 225, the preparation including netarsudil dimesylate, betaxolol mesylate and bimatoprost has good stability after being stored under a storage condition of 40° C. for 14 days, which meets a requirement for departing from a storage condition of the commercially available eye drops (Betoptic®, Rhopress®, and Lumigan®) for a short time.

### Embodiments 266 to 270: A Preparation Including Netarsudil Dihydrochloride, Betaxolol Hydrochloride and Bimatoprost

TABLE 375
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 226 | | 227 | | 228 | | 229 | | 230 | |
| 1. | Netarsudil | 11.6 | mg | 11.6 | mg | 23.2 | mg | 23.2 | mg | 46.4 | mg |
| | dihydrochloride | | | | | | | | | | |
| 2. | Betaxolol | 139.8 | mg | 279.6 | mg | 139.8 | mg | 279.6 | mg | 559.3 | mg |
| | hydrochloride | | | | | | | | | | |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 4800 | mg | 4800 | mg | 4800 | mg | 4800 | mg | 4800 | mg |
| 6. | Benzalkonium | 15 | mg | 15 | mg | 15 | mg | 15 | mg | 15 | mg |
| | chloride | | | | | | | | | | |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |
| | added by | | | | | | | | | | |

A preparation process may refer to the preparation process in Embodiments 83 to 87.

TABLE 376
Contents of various main medicine components in Embodiments
266 to 270 after being stored at 5° C. for 24 months

| | | Embodiment | Embodiment | Embodiment | Embodiment | Embodiment |
|---|---|---|---|---|---|---|
| | | 226 | 227 | 228 | 229 | 230 |
| No. | Component | Content % | | | | |
| 1. | Netarsudil | 98.4 | 99.5 | 98.6 | 99.8 | 98.3 |
| | dihydrochloride | | | | | |
| 2. | Betaxolol | 100.6 | 101.3 | 101.2 | 100.3 | 100.8 |
| | hydrochloride | | | | | |
| 3. | Bimatoprost | 101.2 | 100.4 | 99.9 | 100.3 | 101.5 |

According to Embodiments 266 to 270, the preparation including netarsudil dihydrochloride, betaxolol hydrochloride and bimatoprost has good stability after being stored under a storage condition of 5° C. for 24 months, which meets a requirement for a storage condition of the commercially available eye drops (Betoptic®, Rhopress®, and Lumigan®) before opening.

TABLE 377
Contents of various main medicine components in Embodiments
266 to 270 after being stored at 25° C. for 6 weeks

| | | Embodiment | Embodiment | Embodiment | Embodiment | Embodiment |
|---|---|---|---|---|---|---|
| | | 226 | 227 | 228 | 229 | 230 |
| No. | Component | Content % | | | | |
| 1. | Netarsudil | 100.2 | 100.5 | 99.7 | 101.3 | 99.7 |
| | dihydrochloride | | | | | |
| 2. | Betaxolol | 100.2 | 101.5 | 100.1 | 99.8 | 100.2 |
| | hydrochloride | | | | | |
| 3. | Bimatoprost | 99.5 | 100.9 | 99.0 | 100.5 | 99.2 |

According to Embodiments 266 to 270, the preparation including netarsudil dihydrochloride, betaxolol hydrochloride and bimatoprost has good stability after being stored under a storage condition of 25° C. for 6 weeks, which meets a requirement for a storage condition of the commercially available eye drops (Betoptic®, Rhopress®, and Lumigan®) after opening.

TABLE 378
Contents of various main medicine components in Embodiments
266 to 270 after being stored at 40° C. for 14 days

| | | Embodiment | Embodiment | Embodiment | Embodiment | Embodiment |
|---|---|---|---|---|---|---|
| | | 226 | 227 | 228 | 229 | 230 |
| No. | Component | Content % | | | | |
| 1. | Netarsudil | 100.2 | 99.7 | 100.8 | 99.7 | 100.2 |
| | dihydrochloride | | | | | |
| 2. | Betaxolol | 98.9 | 100.2 | 99.4 | 100.9 | 99.3 |
| | hydrochloride | | | | | |
| 3. | Bimatoprost | 100.3 | 98.4 | 101.2 | 99.0 | 100.2 |

According to Embodiments 266 to 270, the preparation including netarsudil dihydrochloride, betaxolol hydrochloride and bimatoprost has good stability after being stored under a storage condition of 40° C. for 14 days, which meets a requirement for departing from a storage condition of the commercially available eye drops (Betoptic®, Rhopress®, and Lumigan®) for a short time.

### Embodiments 231 to 235: A Preparation Including Netarsudil Dimesylate, Betaxolol Mesylate and Travoprost

TABLE 379
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 231 | | 232 | | 233 | | 234 | | 235 | |
| 1. | Netarsudil dimesylate | 14.2 | mg | 14.2 | mg | 28.5 | mg | 28.5 | mg | 57.0 | mg |
| 2. | Betaxolol mesylate | 164.1 | mg | 328.2 | mg | 164.1 | mg | 328.2 | mg | 656.3 | mg |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 4800 | mg | 4800 | mg | 4800 | mg | 4800 | mg | 4800 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection added | 100 | mL | 100 | mL | 100 | mL | 100 | mL | 100 | mL |
| | by | | | | | | | | | | |

A preparation process may refer to the preparation process in Embodiments 83 to 87.

TABLE 380
Contents of various main medicine components in Embodiments
231 to 235 after being stored at 5° C. for 24 months

| | | Embodiment | Embodiment | Embodiment | Embodiment | Embodiment |
|---|---|---|---|---|---|---|
| | | 231 | 232 | 233 | 234 | 235 |
| No. | Component | Content % | | | | |
| 1. | Netarsudil dimesylate | 99.8 | 100.2 | 99.1 | 100.1 | 100.4 |
| 2. | Betaxolol mesylate | 100.2 | 99.8 | 100.8 | 99.9 | 99.3 |
| 3. | Travoprost | 99.4 | 99.6 | 99.9 | 99.8 | 99.9 |

TABLE 381
Contents of various main medicine components in commercially
available eye drops (Betoptic ®, Rhopress ®, and
Travatan ®) after being stored at 5° C. for 24 months

| | | Betoptic ® | Rhopress | Travatan ® |
|---|---|---|---|---|
| No. | Component | Content % | | |
| 1. | Betaxolol hydrochloride | 99.9 | — | — |
| 2. | Netarsudil dimesylate | — | 99.9 | — |
| 3. | Travoprost | — | — | 99.8 |

According to Embodiments 231 to 235, the preparation including netarsudil dimesylate, betaxolol mesylate and travoprost has good stability after being stored under a storage condition of 5° C. for 24 months, which meets a requirement for a storage condition of the commercially available eye drops (Betoptic®, Rhopress®, and Travatan®) before opening.

TABLE 382
Contents of various main medicine components in Embodiments
231 to 235 after being stored at 25° C. for 6 weeks

| | | Embodiment | Embodiment | Embodiment | Embodiment | Embodiment |
|---|---|---|---|---|---|---|
| | | 231 | 232 | 233 | 234 | 235 |
| No. | Component | Content % | | | | |
| 1. | Netarsudil dimesylate | 100.5 | 100.9 | 99.7 | 100.4 | 99.9 |
| 2. | Betaxolol mesylate | 99.5 | 99.4 | 99.8 | 100.4 | 100.3 |
| 3. | Travoprost | 100.0 | 99.9 | 99.6 | 100.9 | 100.6 |

TABLE 383
Contents of various main medicine components in commercially
available eye drops (Betoptic ®, Rhopress ®, and
Travatan ®) after being stored at 25° C. for 6 weeks

| | | Betoptic ® | Rhopress ® | Travatan ® |
|---|---|---|---|---|
| No. | Component | Content % | | |
| 1. | Betaxolol hydrochloride | 99.7 | — | — |
| 2. | Netarsudil dimesylate | — | 99.8 | — |
| 3. | Travoprost | — | — | 99.5 |

According to Embodiments 231 to 235, the preparation including netarsudil dimesylate, betaxolol mesylate and travoprost has good stability after being stored under a storage condition of 25° C. for 6 weeks, which meets a requirement for a storage condition of the commercially available eye drops (Betoptic®, Rhopress®, and Travatan®) after opening.

TABLE 384
Contents of various main medicine components in Embodiments
231 to 235 after being stored at 40° C. for 14 days

| | | Embodiment | Embodiment | Embodiment | Embodiment | Embodiment |
|---|---|---|---|---|---|---|
| | | 231 | 232 | 233 | 234 | 235 |
| No. | Component | Content % | | | | |
| 1. | Netarsudil dimesylate | 99.7 | 99.7 | 100.6 | 99.5 | 99.9 |
| 2. | Betaxolol mesylate | 99.6 | 100.7 | 99.9 | 99.9 | 99.7 |
| 3. | Travoprost | 99.5 | 99.4 | 100.9 | 100.9 | 99.6 |

TABLE 385
Contents of various main medicine components in commercially
available eye drops (Betoptic ®, Rhopress ®, and
Travatan ®) after being stored at 40° C. for 14 days

| | | Betoptic ® | Rhopress ® | Travatan ® |
|---|---|---|---|---|
| No. | Component | Content % | | |
| 1. | Betaxolol hydrochloride | 100.1 | — | — |
| 2. | Netarsudil dimesylate | — | 99.5 | — |
| 3. | Travoprost | — | — | 99.7 |

According to Embodiments 231 to 235, the preparation including netarsudil dimesylate, betaxolol mesylate and travoprost has good stability after being stored under a storage condition of 40° C. for 14 days, which meets a requirement for departing from a storage condition of the commercially available eye drops (Betoptic®, Rhopress®, and Travatan®) for a short time.

### Embodiments 236 to 240: A Preparation Including Netarsudil Dihydrochloride, Betaxolol Hydrochloride and Travoprost

TABLE 386
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 236 | | 237 | | 238 | | 239 | | 240 | |
| 1. | Netarsudil | 11.6 | mg | 11.6 | mg | 23.2 | mg | 23.2 | mg | 46.4 | mg |
| | dihydrochloride | | | | | | | | | | |
| 2. | Betaxolol | 139.8 | mg | 279.6 | mg | 139.8 | mg | 279.6 | mg | 559.3 | mg |
| | hydrochloride | | | | | | | | | | |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 4800 | mg | 4800 | mg | 4800 | mg | 4800 | mg | 4800 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 387
Contents of various main medicine components in Embodiments
236 to 240 after being stored at 5° C. for 24 months

| | | Embodiment | Embodiment | Embodiment | Embodiment | Embodiment |
|---|---|---|---|---|---|---|
| | | 236 | 237 | 238 | 239 | 240 |
| No. | Component | Content % | | | | |
| 1. | Netarsudil | 99.9 | 99.4 | 100.7 | 99.3 | 100.2 |
| | dihydrochloride | | | | | |
| 2. | Betaxolol | 100.6 | 99.5 | 100.0 | 99.5 | 101.3 |
| | hydrochloride | | | | | |
| 3. | Travoprost | 99.7 | 100.8 | 100.6 | 100.3 | 99.7 |

According to Embodiments 236 to 240, the preparation including netarsudil dihydrochloride, betaxolol hydrochloride and travoprost has good stability after being stored under a storage condition of 5° C. for 24 months, which meets a requirement for a storage condition of the commercially available eye drops (Betoptic®, Rhopress®, and Travatan®) before opening.

TABLE 388
Contents of various main medicine components in Embodiments
236 to 240 after being stored at 25° C. for 6 weeks

| | | Embodiment | Embodiment | Embodiment | Embodiment | Embodiment |
|---|---|---|---|---|---|---|
| | | 236 | 237 | 238 | 239 | 240 |
| No. | Component | Content % | | | | |
| 1. | Netarsudil | 100.6 | 98.5 | 100.5 | 99.8 | 99.6 |
| | dihydrochloride | | | | | |
| 2. | Betaxolol | 99.8 | 100.9 | 100.7 | 100.7 | 100.7 |
| | hydrochloride | | | | | |
| 3. | Travoprost | 99.3 | 99.7 | 99.9 | 99.5 | 99.5 |

According to Embodiments 236 to 240, the preparation including netarsudil dihydrochloride, betaxolol hydrochloride and travoprost has good stability after being stored under a storage condition of 25° C. for 6 weeks, which meets a requirement for a storage condition of the commercially available eye drops (Betoptic®, Rhopress®, and Travatan®) after opening.

TABLE 389
Contents of various main medicine components in Embodiments
236 to 240 after being stored at 40° C. for 14 days

| | | Embodiment | Embodiment | Embodiment | Embodiment | Embodiment |
|---|---|---|---|---|---|---|
| | | 236 | 237 | 238 | 239 | 240 |
| No. | Component | Content % | | | | |
| 1. | Netarsudil | 99.6 | 99.9 | 99.2 | 100.4 | 99.9 |
| | dihydrochloride | | | | | |
| 2. | Betaxolol | 100.9 | 100.7 | 100.3 | 99.7 | 99.5 |
| | hydrochloride | | | | | |
| 3. | Travoprost | 100.6 | 99.7 | 100.3 | 100.3 | 100.6 |

According to Embodiments 236 to 240, the preparation including netarsudil dihydrochloride, betaxolol hydrochloride and travoprost has good stability after being stored under a storage condition of 40° C. for 14 days, which meets a requirement for departing from a storage condition of the commercially available eye drops (Betoptic®, Rhopress®, and Travatan®) for a short time.

### Embodiments 241 to 245: A Preparation Including Netarsudil Dimesylate, Betaxolol Mesylate and Tafluprost

TABLE 390
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 241 | | 242 | | 243 | | 344 | | 345 | |
| 1. | Netarsudil dimesylate | 14.2 | mg | 14.2 | mg | 28.5 | mg | 28.5 | mg | 57.0 | mg |
| 2. | Betaxolol mesylate | 164.1 | mg | 328.2 | mg | 164.1 | mg | 328.2 | mg | 656.3 | mg |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 4800 | mg | 4800 | mg | 4800 | mg | 4800 | mg | 4800 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 391
Contents of various main medicine components in Embodiments
241 to 245 after being stored at 5° C. for 24 months

| | | Embodiment | Embodiment | Embodiment | Embodiment | Embodiment |
|---|---|---|---|---|---|---|
| | | 241 | 242 | 243 | 344 | 345 |
| No. | Component | Content % | | | | |
| 1. | Netarsudil dimesylate | 100.6 | 99.3 | 99.8 | 99.5 | 100.3 |
| 2. | Betaxolol mesylate | 100.9 | 99.3 | 100.3 | 100.4 | 99.6 |
| 3. | Tafluprost | 99.7 | 99.9 | 100.5 | 100.4 | 100.3 |

TABLE 392
Contents of various main medicine components in commercially
available eye drops (Betoptic ®, Rhopress ®, and
Tapros ®) after being stored at 5° C. for 24 months

| | | Betoptic ® | Rhopress ® | Tapros ® |
|---|---|---|---|---|
| No. | Component | Content % | | |
| 1. | Betaxolol hydrochloride | 99.9 | — | — |
| 2. | Netarsudil dimesylate | — | 99.9 | — |
| 3. | Tafluprost | — | — | 99.4 |

According to Embodiments 241 to 245, the preparation including netarsudil dimesylate, betaxolol mesylate and tafluprost has good stability after being stored under a storage condition of 5° C. for 24 months, which meets a requirement for a storage condition of the commercially available eye drops (Betoptic®, Rhopress®, and Tapros®) before opening.

TABLE 393
Contents of various main medicine components in Embodiments
241 to 245 after being stored at 25° C. for 6 weeks

| | | Embodiment | Embodiment | Embodiment | Embodiment | Embodiment |
|---|---|---|---|---|---|---|
| | | 241 | 242 | 243 | 344 | 345 |
| No. | Component | Content % | | | | |
| 1. | Netarsudil dimesylate | 99.4 | 100.5 | 101.7 | 100.8 | 100.1 |
| 2. | Betaxolol mesylate | 99.6 | 100.2 | 99.5 | 99.5 | 100.4 |
| 3. | Tafluprost | 99.7 | 99.9 | 99.9 | 99.8 | 100.7 |

TABLE 394
Contents of various main medicine components in commercially
available eye drops (Betoptic ®, Rhopress ®, and
Tapros ®) after being stored at 25° C. for 6 weeks

| | | Betoptic ® | Rhopress ® | Tapros ® |
|---|---|---|---|---|
| No. | Component | Content % | | |
| 1. | Betaxolol | 99.7 | — | — |
| | hydrochloride | | | |
| 2. | Netarsudil | — | 99.8 | — |
| | dimesylate | | | |
| 3. | Tafluprost | — | — | 99.3 |

According to Embodiments 241 to 245, the preparation including netarsudil dimesylate, betaxolol mesylate and tafluprost has good stability after being stored under a storage condition of 25° C. for 6 weeks, which meets a requirement for a storage condition of the commercially available eye drops (Betoptic®, Rhopress®, and Tapros®) after opening.

TABLE 395
Contents of various main medicine components in Embodiments
241 to 245 after being stored at 40° C. for 14 days

| | | Embodiment | Embodiment | Embodiment | Embodiment | Embodiment |
|---|---|---|---|---|---|---|
| | | 241 | 242 | 243 | 344 | 345 |
| No. | Component | Content % | | | | |
| 1. | Netarsudil dimesylate | 99.7 | 99.8 | 99.9 | 99.2 | 100.3 |
| 2. | Betaxolol mesylate | 99.7 | 100.3 | 100.2 | 100.6 | 100.0 |
| 3. | Tafluprost | 99.8 | 100.2 | 100.6 | 100.7 | 100.5 |

TABLE 396
Contents of various main medicine components in commercially
available eye drops (Betoptic ®, Rhopress ®, and
Tapros ®) after being stored at 40° C. for 14 days

| | | Betoptic ® | Rhopress ® | Tapros ® |
|---|---|---|---|---|
| No. | Component | Content % | | |
| 1. | Betaxolol | 100.1 | — | — |
| | hydrochloride | | | |
| 2. | Netarsudil | — | 99.5 | — |
| | dimesylate | | | |
| 3. | Tafluprost | — | — | 99.8 |

According to Embodiments 241 to 245, the preparation including netarsudil dimesylate, betaxolol mesylate and tafluprost has good stability after being stored under a storage condition of 40° C. for 14 days, which meets a requirement for departing from a storage condition of the commercially available eye drops (Betoptic®, Rhopress®, and Tapros®) for a short time.

### Embodiments 246 to 250: A Preparation Including Netarsudil Dihydrochloride, Betaxolol Hydrochloride and Tafluprost

TABLE 397
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 246 | | 247 | | 248 | | 249 | | 250 | |
| 1. | Netarsudil | 11.6 | mg | 11.6 | mg | 23.2 | mg | 23.2 | mg | 46.4 | mg |
| | dihydrochloride | | | | | | | | | | |
| 2. | Betaxolol | 139.8 | mg | 279.6 | mg | 139.8 | mg | 279.6 | mg | 559.3 | mg |
| | hydrochloride | | | | | | | | | | |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 4800 | mg | 4800 | mg | 4800 | mg | 4800 | mg | 4800 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 398
Contents of various main medicine components in Embodiments
246 to 250 after being stored at 5° C. for 24 months

| | | Embodiment | Embodiment | Embodiment | Embodiment | Embodiment |
|---|---|---|---|---|---|---|
| | | 246 | 247 | 248 | 249 | 250 |
| No. | Component | Content % | | | | |
| 1. | Netarsudil | 99.6 | 100.7 | 100.5 | 100.9 | 99.7 |
| | dihydrochloride | | | | | |
| 2. | Betaxolol | 100.9 | 100.4 | 100.5 | 100.4 | 100.5 |
| | hydrochloride | | | | | |
| 3. | Tafluprost | 99.2 | 100.1 | 101.5 | 100.5 | 100.3 |

According to Embodiments 246 to 250, the preparation including netarsudil dihydrochloride, betaxolol hydrochloride and tafluprost has good stability after being stored under a storage condition of 5° C. for 24 months, which meets a requirement for a storage condition of the commercially available eye drops (Betoptic®, Rhopress®, and Tapros®) before opening.

TABLE 399
Contents of various main medicine components in Embodiments
246 to 250 after being stored at 25° C. for 6 weeks

| | | Embodiment | Embodiment | Embodiment | Embodiment | Embodiment |
|---|---|---|---|---|---|---|
| | | 246 | 247 | 248 | 249 | 250 |
| No. | Component | Content % | | | | |
| 1. | Netarsudil | 99.7 | 100.9 | 100.2 | 99.8 | 100.6 |
| | dihydrochloride | | | | | |
| 2. | Betaxolol | 100.6 | 99.9 | 100.2 | 100.6 | 99.5 |
| | hydrochloride | | | | | |
| 3. | Tafluprost | 100.4 | 100.0 | 99.3 | 99.9 | 99.4 |

According to Embodiments 246 to 250, the preparation including netarsudil dihydrochloride, betaxolol hydrochloride and tafluprost has good stability after being stored under a storage condition of 25° C. for 6 weeks, which meets a requirement for a storage condition of the commercially available eye drops (Betoptic®, Rhopress®, and Tapros®) after opening.

TABLE 400
Contents of various main medicine components in Embodiments
246 to 250 after being stored at 40° C. for 14 days

| | | Embodiment | Embodiment | Embodiment | Embodiment | Embodiment |
|---|---|---|---|---|---|---|
| | | 246 | 247 | 248 | 249 | 250 |
| No. | Component | Content % | | | | |
| 1. | Netarsudil | 100.3 | 100.5 | 100.5 | 99.4 | 100.9 |
| | dihydrochloride | | | | | |
| 2. | Betaxolol | 99.2 | 100.8 | 101.7 | 99.6 | 100.7 |
| | hydrochloride | | | | | |
| 3. | Tafluprost | 100.7 | 100.8 | 100.2 | 100.3 | 99.5 |

According to Embodiments 246 to 230, the preparation including netarsudil dihydrochloride, betaxolol hydrochloride and tafluprost has good stability after being stored under a storage condition of 40° C. for 14 days, which meets a requirement for departing from a storage condition of the commercially available eye drops (Betoptic®, Rhopress®, and Tapros®) for a short time.

### Comparative Examples 89 to 92: A Preparation Including Netarsudil Dimesylate, Betaxolol Hydrochloride and Latanoprost

TABLE 401
Preparation of a local ophthalmic medicine composition solution for reducing
an intra-ocular pressure through a conventional method as follows:

| | | Comparative | | Comparative | | Comparative | | Comparative | |
|---|---|---|---|---|---|---|---|---|---|
| No. | Component | example 89 | | example 90 | | example 91 | | example 92 | |
| 1. | Netarsudil dimesylate | 14.2 | mg | 14.2 | mg | 28.5 | mg | 28.5 | mg |
| 2. | Betaxolol hydrochloride | 139.8 | mg | 279.6 | mg | 139.8 | mg | 279.6 | mg |
| 3. | Latanoprost | 5 | mg | 5 | mg | 5 | mg | 5 | mg |
| 4. | Boric acid | 50 | mg | 50 | mg | 50 | mg | 50 | mg |
| 5. | Mannitol | 4800 | mg | 4800 | mg | 4800 | mg | 4800 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 402
Contents of various main medicine components in Comparative
examples 89 to 92 after being stored at 5° C. for 7 days

| | | Comparative | Comparative | Comparative | Comparative |
|---|---|---|---|---|---|
| | | example 89 | example 90 | example 91 | example 92 |
| No. | Component | Content % | | | |
| 1. | Netarsudil dimesylate | <30 | <30 | <30 | <30 |
| 2. | Betaxolol hydrochloride | 100.9 | 101.8 | 100.4 | 100.6 |
| 3. | Latanoprost | 100.7 | 99.7 | 99.1 | 99.7 |

According to Comparative examples 89 to 92, the preparation including netarsudil dimesylate, betaxolol hydrochloride and latanoprost has incompatibility under the storage condition of 5° C., and netarsudil dimesylate is precipitated.

### Comparative Examples 117 to 120: A Preparation Including Netarsudil Dimesylate, Betaxolol Hydrochloride and Bimatoprost

TABLE 403
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 93 | | example 94 | | example 95 | | example 96 | |
| 1. | Netarsudil dimesylate | 14.2 | mg | 14.2 | mg | 28.5 | mg | 28.5 | mg |
| 2. | Betaxolol hydrochloride | 139.8 | mg | 279.6 | mg | 139.8 | mg | 279.6 | mg |
| 3. | Bimatoprost | 30 | mg | 30 | mg | 30 | mg | 30 | mg |
| 4. | Boric acid | 50 | mg | 50 | mg | 50 | mg | 50 | mg |
| 5. | Mannitol | 4800 | mg | 4800 | mg | 4800 | mg | 4800 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 404
Contents of various main medicine components in Comparative
examples 117 to 120 after being stored at 5° C. for 7 days

| | | Comparative | Comparative | Comparative | Comparative |
|---|---|---|---|---|---|
| | | example 93 | example 94 | example 95 | example 96 |
| No. | Component | Content % | | | |
| 1. | Netarsudil dimesylate | <30 | <30 | <30 | <30 |
| 2. | Betaxolol hydrochloride | 100.5 | 100.2 | 100.5 | 100.6 |
| 3. | Bimatoprost | 99.2 | 101.5 | 100.3 | 99.7 |

According to Comparative examples 117 to 120, the preparation including netarsudil dimesylate, betaxolol hydrochloride and bimatoprost has incompatibility under the storage condition of 5° C., and netarsudil dimesylate is precipitated.

### Comparative Examples 97 to 100: A Preparation Including Netarsudil Dimesylate, Betaxolol Hydrochloride and Travoprost

TABLE 405
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 97 | | example 98 | | example 99 | | example 100 | |
| 1. | Netarsudil dimesylate | 14.2 | mg | 14.2 | mg | 28.5 | mg | 28.5 | mg |
| 2. | Betaxolol hydrochloride | 139.8 | mg | 279.6 | mg | 139.8 | mg | 279.6 | mg |
| 3. | Travoprost | 4 | mg | 4 | mg | 4 | mg | 4 | mg |
| 4. | Boric acid | 50 | mg | 50 | mg | 50 | mg | 50 | mg |
| 5. | Mannitol | 4800 | mg | 4800 | mg | 4800 | mg | 4800 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 406
Contents of various main medicine components in Comparative
examples 97 to 100 after being stored at 5° C. for 7 days

| | | Comparative | Comparative | Comparative | Comparative |
|---|---|---|---|---|---|
| | | example 97 | example 98 | example 99 | example 100 |
| No. | Component | Content % | | | |
| 1. | Netarsudil dimesylate | <30 | <30 | <30 | <30 |
| 2. | Betaxolol hydrochloride | 100.3 | 100.2 | 100.6 | 100.7 |
| 3. | Travoprost | 99.0 | 99.9 | 99.4 | 99.8 |

According to Comparative examples 97 to 100, the preparation including netarsudil dimesylate, betaxolol hydrochloride and travoprost has incompatibility under the storage condition of 5° C., and netarsudil dimesylate is precipitated.

### Comparative Examples 101 to 104: A Preparation Including Netarsudil Dimesylate, Betaxolol Hydrochloride and Tafluprost

TABLE 407
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 101 | | example 102 | | example 103 | | example 104 | |
| 1. | Netarsudil dimesylate | 14.2 | mg | 14.2 | mg | 28.5 | mg | 28.5 | mg |
| 2. | Betaxolol hydrochloride | 139.8 | mg | 279.6 | mg | 139.8 | mg | 279.6 | mg |
| 3. | Tafluprost | 1.5 | mg | 1.5 | mg | 1.5 | mg | 1.5 | mg |
| 4. | Boric acid | 50 | mg | 50 | mg | 50 | mg | 50 | mg |
| 5. | Mannitol | 4800 | mg | 4800 | mg | 4800 | mg | 4800 | mg |
| 6. | Benzalkonium chloride | 20 | mg | 20 | mg | 20 | mg | 20 | mg |
| 7. | Sodium hydroxide | q.s. | | q.s | | q.s. | | q.s. | |
| 8. | Water for injection added | 100 | mL | 100 | mL | 100 | mL | 100 | mL |

by

A preparation process may re er to the preparation process in Embodiments 83 to 87.

TABLE 408
Contents of various main medicine components in Comparative
examples 101 to 104 after being stored at 5° C. for 7 days

| | | Comparative | Comparative | Comparative | Comparative |
|---|---|---|---|---|---|
| | | example 101 | example 102 | example 103 | example 104 |
| No. | Component | Content % | | | |
| 1. | Netarsudil dimesylate | <30 | <30 | <30 | <30 |
| 2. | Betaxolol hydrochloride | 100.5 | 100.1 | 99.3 | 100.8 |
| 3. | Tafluprost | 99.3 | 99.9 | 99.5 | 99.5 |

According to Comparative examples 101 to 104, the preparation including netarsudil dimesylate, betaxolol hydrochloride and tafluprost has incompatibility under the storage condition of 5° C., and netarsudil dimesylate is precipitated.

### Embodiments 251 to 255: A Preparation Including Netarsudil Dimesylate, Metipranolol Mesylate and Latanoprost

TABLE 409
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 251 | | 252 | | 253 | | 254 | | 255 | |
| 1. | Netarsudil dimesylate | 14.2 | mg | 14.2 | mg | 28.5 | mg | 28.5 | mg | 57.0 | mg |
| 2. | Metipranolol mesylate | 131.1 | mg | 393.2 | mg | 131.1 | mg | 393.2 | mg | 786.4 | mg |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 410
Contents of various main medicine components in Embodiments
251 to 255 after being stored at 5° C. for 24 months

| | | Embodiment | Embodiment | Embodiment | Embodiment | Embodiment |
|---|---|---|---|---|---|---|
| | | 251 | 252 | 253 | 254 | 255 |
| No. | Component | Content % | | | | |
| 1. | Netarsudil dimesylate | 100.4 | 99.7 | 100.4 | 100.9 | 99.0 |
| 2. | Metipranolol mesylate | 100.2 | 100.5 | 100.2 | 100.3 | 99.6 |
| 3. | Latanoprost | 100.6 | 99.5 | 100.5 | 100.5 | 100.2 |

TABLE 411
Contents of various main medicine components in commercially
available eye drops (Optipranolol ®, Rhopress ®, and
Xalatan ®) after being stored at 5° C. for 24 months

| | | Optipranolol ® | Rhopress ® | Xalatan ® |
|---|---|---|---|---|
| No. | Component | Content % | | |
| 1. | Metipranolol | 99.8 | — | — |
| | hydrochloride | | | |
| 2. | Netarsudil | — | 99.9 | — |
| | dimesylate | | | |
| 3. | Latanoprost | — | — | 99.8 |

According to Embodiments 251 to 255, the preparation including netarsudil dimesylate, metipranolol mesylate and latanoprost has good stability after being stored under a storage condition of 5° C. for 24 months, which meets a requirement for a storage condition of the commercially available eye drops (Optipranolol®, Rhopress®, and Xalatan®) before opening.

TABLE 412
Contents of various main medicine components in Embodiments
251 to 255 after being stored at 25° C. for 6 weeks

| | | Embodiment | Embodiment | Embodiment | Embodiment | Embodiment |
|---|---|---|---|---|---|---|
| | | 251 | 252 | 253 | 254 | 255 |
| No. | Component | Content % | | | | |
| 1. | Netarsudil dimesylate | 100.6 | 99.5 | 100.9 | 100.1 | 99.5 |
| 2. | Metipranolol mesylate | 100.3 | 101.3 | 101.1 | 101.4 | 100.4 |
| 3. | Latanoprost | 100.1 | 100.8 | 100.7 | 99.6 | 100.2 |

TABLE 413
Contents of various main medicine components in commercially
available eye drops (Optipranolol ®, Rhopress ®, and
Xalatan ®) after being stored at 25° C. for 6 weeks

| | | Optipranolol ® | Rhopress ® | Xalatan ® |
|---|---|---|---|---|
| No. | Component | Content % | | |
| 1. | Metipranolol | 99.7 | — | — |
| | hydrochloride | | | |
| 2. | Netarsudil | — | 99.8 | — |
| | dimesylate | | | |
| 3. | Latanoprost | — | — | 99.6 |

According to Embodiments 251 to 255, the preparation including netarsudil dimesylate, metipranolol mesylate and latanoprost has good stability after being stored under a storage condition of 25° C. for 6 weeks, which meets a requirement for a storage condition of the commercially available eye drops (Optipranolol®, Rhopress®, and Xalatan®) after opening.

TABLE 414
Contents of various main medicine components in Embodiments
251 to 255 after being stored at 40° C. for 14 days

| | | Embodiment | Embodiment | Embodiment | Embodiment | Embodiment |
|---|---|---|---|---|---|---|
| | | 251 | 252 | 253 | 254 | 255 |
| No. | Component | Content % | | | | |
| 1. | Netarsudil dimesylate | 99.6 | 99.7 | 99.8 | 99.9 | 99.8 |
| 2. | Metipranolol mesylate | 100.7 | 100.1 | 99.9 | 100.5 | 100.9 |
| 3. | Latanoprost | 99.8 | 99.4 | 99.6 | 100.9 | 99.7 |

TABLE 415
Contents of various main medicine components in commercially
available eye drops (Optipranolol ®, Rhopress ®, and
Xalatan ®) after being stored at 40° C. for 14 days

| | | Optipranolol ® | Rhopress ® | Xalatan ® |
|---|---|---|---|---|
| No. | Component | Content % | | |
| 1. | Metipranolol | 99.6 | — | — |
| | hydrochloride | | | |
| 2. | Netarsudil | — | 99.5 | — |
| | dimesylate | | | |
| 3. | Latanoprost | — | — | 99.7 |

According to Embodiments 251 to 255, the preparation including netarsudil dimesylate, metipranolol mesylate and latanoprost has good stability after being stored under a storage condition of 40° C. for 14 days, which meets a requirement for departing from a storage condition of the commercially available eye drops (Optipranolol®, Rhopress®, and Xalatan®) for a short time.

### Embodiments 256 to 260: A Preparation Including Netarsudil Dihydrochloride, Metipranolol Hydrochloride and Latanoprost

TABLE 416
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 256 | | 257 | | 258 | | 259 | | 260 | |
| 1. | Netarsudil | 11.6 | mg | 11.6 | mg | 23.2 | mg | 23.2 | mg | 46.4 | mg |
| | dihydrochloride | | | | | | | | | | |
| 2. | Metipranolol | 111.8 | mg | 335.4 | mg | 111.8 | mg | 335.4 | mg | 670.8 | mg |
| | hydrochloride | | | | | | | | | | |
| 3. | Latanoprost | 5 | mg | 5 | mg | 5 | mg | 5 | mg | 5 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 417
Contents of various main medicine components in Embodiments
256 to 260 after being stored at 5° C. for 24 months

| | | Embodiment | Embodiment | Embodiment | Embodiment | Embodiment |
|---|---|---|---|---|---|---|
| | | 256 | 257 | 258 | 259 | 260 |
| No. | Component | Content % | | | | |
| 1. | Netarsudil | 99.6 | 100.2 | 99.5 | 99.8 | 100.9 |
| | dihydrochloride | | | | | |
| 2. | Metipranolol | 99.2 | 100.7 | 100.1 | 100.7 | 101.9 |
| | hydrochloride | | | | | |
| 3. | Latanoprost | 100.8 | 100.4 | 99.9 | 100.1 | 100.3 |

According to Embodiments 256 to 260, the preparation including netarsudil dihydrochloride, metipranolol hydrochloride and latanoprost has good stability after being stored under a storage condition of 5° C. for 24 months, which meets a requirement for a storage condition of the commercially available eye drops (Optipranolol®, Rhopress®, and Xalatan®) before opening.

TABLE 418
Contents of various main medicine components in Embodiments
256 to 260 after being stored at 25° C. for 6 weeks

| | | Embodiment | Embodiment | Embodiment | Embodiment | Embodiment |
|---|---|---|---|---|---|---|
| | | 256 | 257 | 258 | 259 | 260 |
| No. | Component | Content % | | | | |
| 1. | Netarsudil | 100.3 | 99.1 | 99.7 | 101.1 | 100.9 |
| | dihydrochloride | | | | | |
| 2. | Metipranolol | 100.6 | 99.9 | 99.1 | 100.9 | 100.3 |
| | hydrochloride | | | | | |
| 3. | Latanoprost | 100.5 | 101.4 | 100.0 | 100.2 | 100.4 |

According to Embodiments 256 to 260, the preparation including netarsudil dihydrochloride, metipranolol hydrochloride and latanoprost has good stability after being stored under a storage condition of 25° C. for 6 weeks, which meets a requirement for a storage condition of the commercially available eye drops (Optipranolol® Rhopress®, and Xalatan®) before opening.

TABLE 419
Contents of various main medicine components in Embodiments
256 to 260 after being stored at 40° C. for 14 days

| | | Embodiment | Embodiment | Embodiment | Embodiment | Embodiment |
|---|---|---|---|---|---|---|
| | | 256 | 257 | 258 | 259 | 260 |
| No. | Component | Content % | | | | |
| 1. | Netarsudil | 101.1 | 100.1 | 99.3 | 100.1 | 100.9 |
| | dihydrochloride | | | | | |
| 2. | Metipranolol | 99.3 | 100.6 | 100.3 | 99.8 | 100.3 |
| | hydrochloride | | | | | |
| 3. | Latanoprost | 100.4 | 100.8 | 99.1 | 99.9 | 99.4 |

According to Embodiments 256 to 260, the preparation including netarsudil dihydrochloride, metipranolol hydrochloride and latanoprost has good stability after being stored under a storage condition of 40° C. for 14 days, which meets a requirement for departing from a storage condition of the commercially available eye drops (Optipranolol®, Rhopress®, and Xalatan®) for a short time.

### Embodiments 261 to 265: A Preparation Including Netarsudil Dimesylate, Metipranolol Mesylate and Bimatoprost

TABLE 420
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 261 | | 262 | | 263 | | 264 | | 265 | |
| 1. | Netarsudil dimesylate | 14.2 | mg | 14.2 | mg | 28.5 | mg | 28.5 | mg | 57.0 | mg |
| 2. | Metipranolol mesylate | 131.1 | mg | 393.2 | mg | 131.1 | mg | 393.2 | mg | 786.4 | mg |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 5 | mg | 5 | mg | 5 | mg | 5 | mg | 5 | mg |
| 7. | Sodium hydroxide | q.s | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 421
Contents of various main medicine components in Embodiments
261 to 265 after being stored at 5° C. for 24 months

| | | Embodiment | Embodiment | Embodiment | Embodiment | Embodiment |
|---|---|---|---|---|---|---|
| | | 261 | 262 | 263 | 264 | 265 |
| No. | Component | Content % | | | | |
| 1. | Netarsudil dimesylate | 100.2 | 99.9 | 99.8 | 100.2 | 99.1 |
| 2. | Metipranolol mesylate | 100.3 | 100.8 | 100.2 | 100.8 | 100.1 |
| 3. | Bimatoprost | 98.6 | 100.2 | 100.6 | 100.3 | 99.8 |

TABLE 422
Contents of various main medicine components in commercially
available eye drops (Optipranolol ®, Rhopress ®, and
Lumigan ®) after being stored at 5° C. for 24 months

| | | Optipranolol ® | Rhopress ® | Lumigan ® |
|---|---|---|---|---|
| No. | Component | Content % | | |
| 1. | Metipranolol | 99.8 | — | — |
| | hydrochloride | | | |
| 2. | Netarsudil | — | 99.9 | — |
| | dimesylate | | | |
| 3. | Bimatoprost | — | — | 99.4 |

According to Embodiments 261 to 265, the preparation including netarsudil dimesylate, metipranolol mesylate and bimatoprost has good stability after being stored under a storage condition of 5° C. for 24 months, which meets a requirement for a storage condition of the commercially available eye drops (Optipranolol®, Rhopress®, and Lumigan®) before opening.

TABLE 423
Contents of various main medicine components in Embodiments
261 to 265 after being stored at 25° C. for 6 weeks

| | | Embodiment | Embodiment | Embodiment | Embodiment | Embodiment |
|---|---|---|---|---|---|---|
| | | 261 | 262 | 263 | 264 | 265 |
| No. | Component | Content % | | | | |
| 1. | Netarsudil dimesylate | 100.0 | 100.1 | 99.2 | 99.5 | 99.8 |
| 2. | Metipranolol mesylate | 99.9 | 100.1 | 99.9 | 99.3 | 100.3 |
| 3. | Bimatoprost | 99.8 | 99.8 | 100.3 | 100.9 | 100.2 |

TABLE 424
Contents of various main medicine components in commercially
available eye drops (Optipranolol ®, Rhopress ®, and
Lumigan ®) after being stored at 25° C. for 6 weeks

| | | Optipranolol ® | Rhopress ® | Lumigan ® |
|---|---|---|---|---|
| No. | Component | Content % | | |
| 1. | Metipranolol | 99.7 | — | — |
| | hydrochloride | | | |
| 2. | Netarsudil | — | 99.8 | — |
| | dimesylate | | | |
| 3. | Bimatoprost | — | — | 99.5 |

According to Embodiments 261 to 265, the preparation including netarsudil dimesylate, metipranolol mesylate and bimatoprost has good stability after being stored under a storage condition of 25° C. for 6 weeks, which meets a requirement for a storage condition of the commercially available eye drops (Optipranolol®, Rhopress®, and Lumigan®) after opening.

TABLE 425
Contents of various main medicine components in Embodiments
261 to 265 after being stored at 40° C. for 14 days

| | | Embodiment | Embodiment | Embodiment | Embodiment | Embodiment |
|---|---|---|---|---|---|---|
| | | 261 | 262 | 263 | 264 | 265 |
| No. | Component | Content % | | | | |
| 1. | Netarsudil dimesylate | 99.7 | 99.9 | 99.1 | 99.2 | 98.8 |
| 2. | Metipranolol mesylate | 99.1 | 100.2 | 99.8 | 100.8 | 99.1 |
| 3. | Bimatoprost | 99.2 | 98.9 | 100.5 | 99.5 | 99.2 |

TABLE 426
Contents of various main medicine components in commercially
available eye drops (Optipranolol ®, Rhopress ®, and
Lumigan ®) after being stored at 40° C. for 14 days

| | | Optipranolol ® | Rhopress ® | Lumigan ® |
|---|---|---|---|---|
| No. | Component | Content % | | |
| 1. | Metipranolol | 100.1 | — | — |
| | hydrochloride | | | |
| 2. | Netarsudil | — | 99.5 | — |
| | dimesylate | | | |
| 3. | Bimatoprost | — | — | 99.2 |

According to Embodiments 261 to 265, the preparation including netarsudil dimesylate, metipranolol mesylate and bimatoprost has good stability after being stored under a storage condition of 40° C. for 14 days, which meets a requirement for departing from a storage condition of the commercially available eye drops (Optipranolol®, Rhopress®, and Lumigan®) for a short time.

### Embodiments 266 to 270: A Preparation Including Netarsudil Dihydrochloride, Metipranolol Hydrochloride and Bimatoprost

TABLE 427
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 266 | | 267 | | 268 | | 269 | | 270 | |
| 1. | Netarsudil | 11.6 | mg | 11.6 | mg | 23.2 | mg | 23.2 | mg | 46.4 | mg |
| | dihydrochloride | | | | | | | | | | |
| 2. | Metipranolol | 111.8 | mg | 335.4 | mg | 111.8 | mg | 335.4 | mg | 670.8 | mg |
| | hydrochloride | | | | | | | | | | |
| 3. | Bimatoprost | 30 | mg | 30 | mg | 30 | mg | 30 | mg | 30 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium | 5 | mg | 5 | mg | 5 | mg | 5 | mg | 5 | mg |
| | chloride | | | | | | | | | | |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 428
Contents of various main medicine components in Embodiments
266 to 270 after being stored at 5° C. for 24 months

| | | Embodiment | Embodiment | Embodiment | Embodiment | Embodiment |
|---|---|---|---|---|---|---|
| | | 266 | 267 | 268 | 269 | 270 |
| No. | Component | Content % | | | | |
| 1. | Netarsudil | 99.4 | 99.9 | 99.6 | 99.1 | 99.3 |
| | dihydrochloride | | | | | |
| 2. | Metipranolol | 101.6 | 100.3 | 100.2 | 100.6 | 100.1 |
| | hydrochloride | | | | | |
| 3. | Bimatoprost | 101.1 | 99.4 | 100.9 | 100.5 | 100.5 |

According to Embodiments 266 to 270, the preparation including netarsudil dihydrochloride, metipranolol hydrochloride and bimatoprost has good stability after being stored under a storage condition of 5° C. for 24 months, which meets a requirement for a storage condition of the commercially available eye drops (Optipranolol®, Rhopress®, and Lumigan®) before opening.

TABLE 429
Contents of various main medicine components in Embodiments
266 to 270 after being stored at 25° C. for 6 weeks

| | | Embodiment | Embodiment | Embodiment | Embodiment | Embodiment |
|---|---|---|---|---|---|---|
| | | 266 | 267 | 268 | 269 | 270 |
| No. | Component | Content % | | | | |
| 1. | Netarsudil | 100.1 | 100.3 | 100.7 | 100.3 | 98.7 |
| | dihydrochloride | | | | | |
| 2. | Metipranolol | 100.6 | 100.5 | 100.4 | 99.2 | 100.3 |
| | hydrochloride | | | | | |
| 3. | Bimatoprost | 99.3 | 100.1 | 99.7 | 100.9 | 99.7 |

According to Embodiments 266 to 270, the preparation including netarsudil dihydrochloride, metipranolol hydrochloride and bimatoprost has good stability after being stored under a storage condition of 25° C. for 6 weeks, which meets a requirement for a storage condition of the commercially available eye drops (Optipranolol®, Rhopress®, and Lumigan®) after opening.

TABLE 430
Contents of various main medicine components in Embodiments
266 to 270 after being stored at 40° C. for 14 days

| | | Embodiment | Embodiment | Embodiment | Embodiment | Embodiment |
|---|---|---|---|---|---|---|
| | | 266 | 267 | 268 | 269 | 270 |
| No. | Component | Content % | | | | |
| 1. | Netarsudil | 101.2 | 100.7 | 100.2 | 100.7 | 101.2 |
| | dihydrochloride | | | | | |
| 2. | Metipranolol | 99.9 | 100.6 | 99.3 | 100.1 | 100.3 |
| | hydrochloride | | | | | |
| 3. | Bimatoprost | 100.2 | 100.4 | 100.2 | 100.0 | 100.1 |

According to Embodiments 266 to 270, the preparation including netarsudil dihydrochloride, metipranolol hydrochloride and bimatoprost has good stability after being stored under a storage condition of 40° C. for 14 days, which meets a requirement for departing from a storage condition of the commercially available eye drops (Optipranolol®, Rhopress®, and Lumigan®) for a short time.

### Embodiments 271 to 275: A Preparation Including Netarsudil Dimesylate, Metipranolol Mesylate and Travoprost

TABLE 431
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No | Component | 271 | | 272 | | 273 | | 274 | | 275 | |
| 1. | Netarsudil dimesylate | 14.2 | mg | 14.2 | mg | 28.5 | mg | 28.5 | mg | 57.0 | mg |
| 2. | Metipranolol mesylate | 131.1 | mg | 393.2 | mg | 131.1 | mg | 393.2 | mg | 786.4 | mg |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 432
Contents of various main medicine components in Embodiments
271 to 275 after being stored at 5° C. for 24 months

| | | Embodiment | Embodiment | Embodiment | Embodiment | Embodiment |
|---|---|---|---|---|---|---|
| | | 271 | 272 | 273 | 274 | 275 |
| No. | Component | Content % | | | | |
| 1. | Netarsudil dimesylate | 99.7 | 99.9 | 100.5 | 100.4 | 100.5 |
| 2. | Metipranolol mesylate | 100.4 | 100.8 | 100.7 | 100.3 | 100.4 |
| 3. | Travoprost | 100.4 | 99.5 | 99.3 | 100.8 | 99.4 |

TABLE 433
Contents of various main medicine components in commercially
available eye drops (Optipranolol ®, Rhopress ®, and
Travatan ®) after being stored at 5° C. for 24 months

| | | Optipranolol ® | Rhopress ® | Travatan ® |
|---|---|---|---|---|
| No. | Component | Content % | | |
| 1. | Metipranolol | 99.8 | — | — |
| | hydrochloride | | | |
| 2. | Netarsudil | — | 99.9 | — |
| | dimesylate | | | |
| 3. | Travoprost | — | — | 99.8 |

According to Embodiments 271 to 275, the preparation including netarsudil dimesylate, metipranolol mesylate and travoprost has good stability after being stored under a storage condition of 5° C. for 24 months, which meets a requirement for a storage condition of the commercially available eye drops (Optipranolol®, Rhopress®, and Travatan®) before opening.

TABLE 434
Contents of various main medicine components in Embodiments
271 to 275 after being stored at 25° C. for 6 weeks

| | | Embodiment | Embodiment | Embodiment | Embodiment | Embodiment |
|---|---|---|---|---|---|---|
| | | 271 | 272 | 273 | 274 | 275 |
| No. | Component | Content % | | | | |
| 1. | Netarsudil dimesylate | 100.5 | 99.4 | 100.5 | 100.4 | 100.3 |
| 2. | Metipranolol mesylate | 101.5 | 99.7 | 99.4 | 100.7 | 100.5 |
| 3. | Travoprost | 100.4 | 98.9 | 100.6 | 100.2 | 100.7 |

TABLE 435
Contents of various main medicine components in commercially
available eye drops (Optipranolol ®, Rhopress ®, and
Travatan ®) after being stored at 25° C. for 6 weeks

| | | Optipranolol ® | Rhopress ® | Travatan ® |
|---|---|---|---|---|
| No. | Component | Content % | | |
| 1. | Metipranolol | 99.7 | — | — |
| | hydrochloride | | | |
| 2. | Netarsudil | — | 99.8 | — |
| | dimesylate | | | |
| 3. | Travoprost | — | — | 99.5 |

According to Embodiments 271 to 275, the preparation including netarsudil dimesylate, metipranolol mesylate and travoprost has good stability after being stored under a storage condition of 25° C. for 6 weeks, which meets a requirement for a storage condition of the commercially available eye drops (Optipranolol®, Rhopress®, and Travatan®) after opening.

TABLE 436
Contents of various main medicine components in Embodiments
271 to 275 after being stored at 40° C. for 14 days

| | | Embodiment | Embodiment | Embodiment | Embodiment | Embodiment |
|---|---|---|---|---|---|---|
| | | 271 | 272 | 273 | 274 | 275 |
| No. | Component | Content % | | | | |
| 1. | Netarsudil dimesylate | 100.7 | 99.3 | 100.4 | 100.5 | 99.5 |
| 2. | Metipranolol mesylate | 99.4 | 100.2 | 99.4 | 98.9 | 99.3 |
| 3. | Travoprost | 99.3 | 100.4 | 100.3 | 100.4 | 99.1 |

TABLE 437
Contents of various main medicine components in commercially
available eye drops (Optipranolol ®, Rhopress ®, and
Travatan ®) after being stored at 40° C. for 14 days

| | | Optipranolol ® | Rhopress ® | Travatan ® |
|---|---|---|---|---|
| No. | Component | Content % | | |
| 1. | Metipranolol | 99.6 | — | — |
| | hydrochloride | | | |
| 2. | Netarsudil | — | 99.5 | — |
| | dimesylate | | | |
| 3. | Travoprost | — | — | 99.7 |

According to Embodiments 271 to 275, the preparation including netarsudil dimesylate, metipranolol mesylate and travoprost has good stability after being stored under a storage condition of 40° C. for 14 days, which meets a requirement for departing from a storage condition of the commercially available eye drops (Optipranolol®, Rhopress®, and Travatan®) for a short time.

### Embodiments 276 to 280: A Preparation Including Netarsudil Dihydrochloride, Metipranolol Hydrochloride and Travoprost

TABLE 438
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 276 | | 277 | | 278 | | 279 | | 280 | |
| 1. | Netarsudil | 11.6 | mg | 11.6 | mg | 23.2 | mg | 23.2 | mg | 46.4 | mg |
| | dihydrochloride | | | | | | | | | | |
| 2. | Metipranolol | 111.8 | mg | 335.4 | mg | 111.8 | mg | 335.4 | mg | 670.8 | mg |
| | hydrochloride | | | | | | | | | | |
| 3. | Travoprost | 4 | mg | 4 | mg | 4 | mg | 4 | mg | 4 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 15 | mg | 15 | mg | 15 | mg | 15 | mg | 15 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 439
Contents of various main medicine components in Embodiments
276 to 280 after being stored at 5° C. for 24 months

| | | Embodiment | Embodiment | Embodiment | Embodiment | Embodiment |
|---|---|---|---|---|---|---|
| | | 276 | 277 | 278 | 279 | 280 |
| No. | Component | Content % | | | | |
| 1. | Netarsudil | 99.4 | 99.2 | 100.1 | 99.6 | 100.1 |
| | dihydrochloride | | | | | |
| 2. | Metipranolol | 101.6 | 99.4 | 99.0 | 99.2 | 101.4 |
| | hydrochloride | | | | | |
| 3. | Travoprost | 99.5 | 99.8 | 100.3 | 100.6 | 100.7 |

According to Embodiments 276 to 280, the preparation including netarsudil dihydrochloride, metipranolol hydrochloride and travoprost has good stability after being stored under a storage condition of 5° C. for 24 months, which meets a requirement for a storage condition of the commercially available eye drops (Optipranolol®, Rhopress®, and Travatan®) before opening.

TABLE 440
Contents of various main medicine components in Embodiments
276 to 280 after being stored at 25° C. for 6 weeks

| | | Embodiment | Embodiment | Embodiment | Embodiment | Embodiment |
|---|---|---|---|---|---|---|
| | | 276 | 277 | 278 | 279 | 280 |
| No. | Component | Content % | | | | |
| 1. | Netarsudil | 100.2 | 99.5 | 100.1 | 99.6 | 100.6 |
| | dihydrochloride | | | | | |
| 2. | Metipranolol | 99.4 | 101.9 | 100.5 | 100.3 | 100.2 |
| | hydrochloride | | | | | |
| 3. | Travoprost | 99.1 | 99.3 | 100.9 | 99.7 | 99.6 |

According to Embodiments 276 to 280, the preparation including netarsudil dihydrochloride, metipranolol hydrochloride and travoprost has good stability after being stored under a storage condition of 25° C. for 6 weeks, which meets a requirement for a storage condition of the commercially available eye drops (Optipranolol®, Rhopress®, and Travatan®) after opening.

TABLE 441
Contents of various main medicine components in Embodiments
276 to 280 after being stored at 40° C. for 14 days

| | | Embodiment | Embodiment | Embodiment | Embodiment | Embodiment |
|---|---|---|---|---|---|---|
| | | 276 | 277 | 278 | 279 | 280 |
| No. | Component | Content % | | | | |
| 1. | Netarsudil | 100.6 | 99.5 | 99.9 | 100.6 | 99.4 |
| | dihydrochloride | | | | | |
| 2. | Metipranolol | 100.2 | 100.1 | 100.2 | 99.4 | 99.2 |
| | hydrochloride | | | | | |
| 3. | Travoprost | 100.1 | 98.7 | 100.6 | 101.3 | 101.6 |

According to Embodiments 276 to 280, the preparation including netarsudil dihydrochloride, metipranolol hydrochloride and travoprost has good stability after being stored under a storage condition of 40° C. for 14 days, which meets a requirement for departing from a storage condition of the commercially available eye drops (Optipranolol®, Rhopress®, and Travatan®) for a short time.

### Embodiments 281 to 285: A Preparation Including Netarsudil Dimesylate, Metipranolol Mesylate and Tafluprost

TABLE 442
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 281 | | 282 | | 283 | | 284 | | 285 | |
| 1. | Netarsudil dimesylate | 14.2 | mg | 14.2 | mg | 28.5 | mg | 28.5 | mg | 57.0 | mg |
| 2. | Metipranolol mesylate | 131.1 | mg | 393.2 | mg | 131.1 | mg | 393.2 | mg | 786.4 | mg |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection added | 100 | mL | 100 | mL | 100 | mL | 100 | mL | 100 | mL |

by

A preparation process may refer to the preparation process in Embodiments 83 to 87.

TABLE 443
Contents of various main medicine components in Embodiments
281 to 285 after being stored at 5° C. for 24 months

| | | Embodiment | Embodiment | Embodiment | Embodiment | Embodiment |
|---|---|---|---|---|---|---|
| | | 281 | 282 | 283 | 284 | 285 |
| No. | Component | Content % | | | | |
| 1. | Netarsudil dimesylate | 100.1 | 99.4 | 99.9 | 99.1 | 101.3 |
| 2. | Metipranolol mesylate | 100.5 | 99.4 | 100.1 | 99.4 | 99.4 |
| 3. | Tafluprost | 99.2 | 100.9 | 100.3 | 100.6 | 100.2 |

TABLE 444
Contents of various main medicine components in commercially
available eye drops (Optipranolol ®, Rhopress ®, and
Tapros ®) after being stored at 5° C. for 24 months

| | | Optipranolol ® | Rhopress ® | Tapros ® |
|---|---|---|---|---|
| No. | Component | Content % | | |
| 1. | Metipranolol | 99.8 | — | — |
| | hydrochloride | | | |
| 2. | Netarsudil | — | 99.9 | — |
| | dimesylate | | | |
| 3. | Tafluprost | — | — | 99.4 |

According to Embodiments 281 to 285, the preparation including netarsudil dimesylate, metipranolol mesylate and tafluprost has good stability after being stored under a storage condition of 5° C. for 24 months, which meets a requirement for a storage condition of the commercially available eye drops (Optipranolol®, Rhopress®, and Tapros®) before opening.

TABLE 445
Contents of various main medicine components in Embodiments
281 to 285 after being stored at 25° C. for 6 weeks

| | | Embodiment | Embodiment | Embodiment | Embodiment | Embodiment |
|---|---|---|---|---|---|---|
| | | 281 | 282 | 283 | 284 | 285 |
| No. | Component | Content % | | | | |
| 1. | Netarsudil dimesylate | 100.4 | 101.5 | 101.3 | 100.1 | 99.1 |
| 2. | Metipranolol mesylate | 100.6 | 100.5 | 99.3 | 99.5 | 101.4 |
| 3. | Tafluprost | 99.3 | 99.1 | 99.6 | 99.3 | 100.3 |

TABLE 446
Contents of various main medicine components in commercially
available eye drops (Optipranolol ®, Rhopress ®, and
Tapros ®) after being stored at 25° C. for 6 weeks

| | | Optipranolol ® | Rhopress ® | Tapros ® |
|---|---|---|---|---|
| No. | Component | Content % | | |
| 1. | Metipranolol | 99.7 | — | — |
| | hydrochloride | | | |
| 2. | Netarsudil | — | 99.8 | — |
| | dimesylate | | | |
| 3. | Tafluprost | — | — | 99.3 |

According to Embodiments 281 to 285, the preparation including netarsudil dimesylate, metipranolol mesylate and tafluprost has good stability after being stored under a storage condition of 25° C. for 6 weeks, which meets a requirement for a storage condition of the commercially available eye drops (Optipranolol®, Rhopress®, and Tapros®) after opening.

TABLE 447
Contents of various main medicine components in Embodiments
281 to 285 after being stored at 40° C. for 14 days

| | | Embodiment | Embodiment | Embodiment | Embodiment | Embodiment |
|---|---|---|---|---|---|---|
| | | 281 | 282 | 283 | 284 | 285 |
| No. | Component | Content % | | | | |
| 1. | Netarsudil dimesylate | 99.2 | 99.1 | 99.4 | 99.5 | 100.2 |
| 2. | Metipranolol mesylate | 99.4 | 100.2 | 100.5 | 100.1 | 99.0 |
| 3. | Tafluprost | 99.6 | 99.2 | 100.2 | 100.6 | 100.2 |

TABLE 448
Contents of various main medicine components in commercially
available eye drops (Optipranolol ®, Rhopress ®, and
Tapros ®) after being stored at 40° C. for 14 days

| | | Optipranolol ® | Rhopress ® | Tapros ® |
|---|---|---|---|---|
| No. | Component | Content % | | |
| 1. | Metipranolol | 99.6 | — | — |
| | hydrochloride | | | |
| 2. | Netarsudil | — | 99.5 | — |
| | dimesylate | | | |
| 3. | Tafluprost | — | — | 99.8 |

According to Embodiments 281 to 285, the preparation including netarsudil dimesylate, metipranolol mesylate and tafluprost has good stability after being stored under a storage condition of 40° C. for 14 days, which meets a requirement for departing from a storage condition of the commercially available eye drops (Optipranolol®, Rhopress®, and Tapros®) for a short time.

### Embodiments 286 to 290: A Preparation Including Netarsudil Dihydrochloride, Metipranolol Hydrochloride and Tafluprost

TABLE 449
Preparation of a local ophthalmic medicine composition solution for reducing
an intra-ocular pressure through a conventional method as follows:

| | | Embodiment | | Embodiment | | Embodiment | | Embodiment | | Embodiment | |
|---|---|---|---|---|---|---|---|---|---|---|---|
| No. | Component | 286 | | 287 | | 288 | | 289 | | 290 | |
| 1. | Netarsudil | 11.6 | mg | 11.6 | mg | 23.2 | mg | 23.2 | mg | 46.4 | mg |
| | dihydrochloride | | | | | | | | | | |
| 2. | Metipranolol | 111.8 | mg | 335.4 | mg | 111.8 | mg | 335.4 | mg | 670.8 | mg |
| | hydrochloride | | | | | | | | | | |
| 3. | Tafluprost | 1.5 | mg | 1.5 | mg | 1.5 | mg | 1.5 | mg | 1.5 | mg |
| 4. | Boric acid | 50 | mg | 50 | mg | 50 | mg | 50 | mg | 50 | mg |
| 5. | Mannitol | 4500 | mg | 4500 | mg | 4500 | mg | 4500 | mg | 4500 | mg |
| 6. | Benzalkonium chloride | 20 | mg | 20 | mg | 20 | mg | 20 | mg | 20 | mg |
| 7. | Sodium hydroxide | q.s. | | q.s. | | q.s. | | q.s. | | q.s. | |
| 8. | Water for injection | 100 | mL | 100 | mL | 100 | mL | 100 | mL | 100 | mL |

added by

A preparation process may refer to the preparation process in Embodiments 83 to 87.

TABLE 450
Contents of various main medicine components in Embodiments
286 to 290 after being stored at 5° C. for 24 months

| | | Embodiment | Embodiment | Embodiment | Embodiment | Embodiment |
|---|---|---|---|---|---|---|
| | | 286 | 287 | 288 | 289 | 290 |
| No. | Component | Content % | | | | |
| 1. | Netarsudil | 99.4 | 100.1 | 100.1 | 100.3 | 99.2 |
| | dihydrochloride | | | | | |
| 2. | Metipranolol | 100.6 | 100.2 | 100.6 | 100.3 | 100.1 |
| | hydrochloride | | | | | |
| 3. | Tafluprost | 99.7 | 99.1 | 100.5 | 100.3 | 100.2 |

According to Embodiments 286 to 290, the preparation including netarsudil dihydrochloride, metipranolol hydrochloride and tafluprost has good stability after being stored under a storage condition of 5° C. for 24 months, which meets a requirement for a storage condition of the commercially available eye drops (Optipranolol®, Rhopress®, and Tapros®) before opening.

TABLE 451
Contents of various main medicine components in Embodiments
286 to 290 after being stored at 25° C. for 6 weeks

| | | Embodiment | Embodiment | Embodiment | Embodiment | Embodiment |
|---|---|---|---|---|---|---|
| | | 286 | 287 | 288 | 289 | 290 |
| No. | Component | Content % | | | | |
| 1. | Netarsudil | 99.2 | 100.1 | 99.2 | 99.2 | 100.5 |
| | dihydrochloride | | | | | |
| 2. | Metipranolol | 100.2 | 99.4 | 100.3 | 100.2 | 100.5 |
| | hydrochloride | | | | | |
| 3. | Tafluprost | 100.5 | 100.1 | 99.1 | 100.9 | 99.6 |

According to Embodiments 286 to 290, the preparation including netarsudil dihydrochloride, metipranolol hydrochloride and tafluprost has good stability after being stored under a storage condition of 25° C. for 6 weeks, which meets a requirement for a storage condition of the commercially available eye drops (Optipranolol®, Rhopress®, and Tapros®) after opening.

TABLE 452
Contents of various main medicine components in Embodiments
286 to 290 after being stored at 40° C. for 14 days

| | | Embodiment | Embodiment | Embodiment | Embodiment | Embodiment |
|---|---|---|---|---|---|---|
| | | 286 | 287 | 288 | 289 | 290 |
| No. | Component | Content % | | | | |
| 1. | Netarsudil | 100.1 | 100.3 | 99.5 | 99.1 | 100.3 |
| | dihydrochloride | | | | | |
| 2. | Metipranolol | 100.2 | 100.1 | 100.7 | 99.7 | 100.5 |
| | hydrochloride | | | | | |
| 3. | Tafluprost | 100.3 | 100.4 | 100.4 | 100.5 | 99.6 |

According to Embodiments 286 to 290, the preparation including netarsudil dihydrochloride, metipranolol hydrochloride and tafluprost has good stability after being stored under a storage condition of 40° C. for 14 days, which meets a requirement for departing from a storage condition of the commercially available eye drops (Optipranolol®, Rhopress®, and Tapros®) for a short time.

### Comparative Examples 105 to 108: A Preparation Including Netarsudil Dimesylate, Metipranolol Hydrochloride and Latanoprost

TABLE 453
Preparation of a local ophthalmic medicine composition solution for reducing
an intra-ocular pressure through a conventional method as follows:

| | | Comparative | | Comparative | | Comparative | | Comparative | |
|---|---|---|---|---|---|---|---|---|---|
| No. | Component | example 105 | | example 106 | | example 107 | | example 108 | |
| 1. | Netarsudil dimesylate | 14.2 | mg | 14.2 | mg | 28.5 | mg | 28.5 | mg |
| 2. | Metipranolol hydrochloride | 111.8 | mg | 335.4 | mg | 111.8 | mg | 335.4 | mg |
| 3. | Latanoprost | 5 | mg | 5 | mg | 5 | mg | 5 | mg |
| 4. | Boric acid | 50 | mg | 50 | mg | 50 | mg | 50 | mg |
| 5. | Mannitol | 4500 | mg | 4500 | mg | 4500 | mg | 4500 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 454
Contents of various main medicine components in Comparative
examples 105 to 108 after being stored at 5° C. for 7 days

| | | Comparative | Comparative | Comparative | Comparative |
|---|---|---|---|---|---|
| | | example 105 | example 106 | example 107 | example 108 |
| No. | Component | Content % | | | |
| 1. | Netarsudil dimesylate | <30 | <30 | <30 | <30 |
| 2. | Metipranolol hydrochloride | 100.3 | 101.1 | 100.2 | 99.6 |
| 3. | Latanoprost | 100.9 | 99.6 | 99.2 | 99.3 |

According to Comparative examples 105 to 108: the preparation including netarsudil dimesylate, metipranolol hydrochloride and latanoprost has incompatibility under the storage condition of 5° C., and netarsudil dimesylate is precipitated.

### Comparative Examples 109 to 112: A Preparation Including Netarsudil Dimesylate, Metipranolol Hydrochloride and Bimatoprost

TABLE 455
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 109 | | example 110 | | example 111 | | example 112 | |
| 1. | Netarsudil dimesylate | 14.2 | mg | 14.2 | mg | 28.5 | mg | 28.5 | mg |
| 2. | Metipranolol hydrochloride | 111.8 | mg | 335.4 | mg | 111.8 | mg | 335.4 | mg |
| 3. | Bimatoprost | 30 | mg | 30 | mg | 30 | mg | 30 | mg |
| 4. | Boric acid | 50 | mg | 50 | mg | 50 | mg | 50 | mg |
| 5. | Mannitol | 4500 | mg | 4500 | mg | 4500 | mg | 4500 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 456
Contents of various main medicine components in Comparative
examples 109 to 112 after being stored at 5° C. for 7 days

| | | Comparative | Comparative | Comparative | Comparative |
|---|---|---|---|---|---|
| | | example 109 | example 110 | example 111 | example 112 |
| No. | Component | Content % | | | |
| 1. | Netarsudil dimesylate | <30 | <30 | <30 | <30 |
| 2. | Metipranolol hydrochloride | 99.5 | 100.3 | 101.5 | 100.2 |
| 3. | Bimatoprost | 99.6 | 100.5 | 100.1 | 99.5 |

According to Comparative examples 109 to 112: the preparation including netarsudil dimesylate, metipranolol hydrochloride and bimatoprost has incompatibility under the storage condition of 5° C., and netarsudil dimesylate is precipitated.

### Comparative Examples 113 to 116: A Preparation Including Netarsudil Dimesylate, Metipranolol Hydrochloride and Travoprost

TABLE 457
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 113 | | example 114 | | example 115 | | example 116 | |
| 1. | Netarsudil dimesylate | 14.2 | mg | 14.2 | mg | 28.5 | mg | 28.5 | mg |
| 2. | Metipranolol hydrochloride | 111.8 | mg | 335.4 | mg | 111.8 | mg | 335.4 | mg |
| 3. | Travoprost | 4 | mg | 4 | mg | 4 | mg | 4 | mg |
| 4. | Boric acid | 50 | mg | 50 | mg | 50 | mg | 50 | mg |
| 5. | Mannitol | 4500 | mg | 4500 | mg | 4500 | mg | 4500 | mg |
| 6. | Benzalkonium chloride | 5 | mg | 5 | mg | 5 | mg | 5 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 458
Contents of various main medicine components in Comparative
examples 113 to 116 after being stored at 5° C. for 7 days

| | | Comparative | Comparative | Comparative | Comparative |
|---|---|---|---|---|---|
| | | example 113 | example 114 | example 115 | example 116 |
| No. | Component | Content % | | | |
| 1. | Netarsudil dimesylate | <30 | <30 | <30 | <30 |
| 2. | Metipranolol hydrochloride | 100.1 | 101.2 | 100.5 | 100.2 |
| 3. | Travoprost | 99.0 | 99.3 | 99.1 | 100.8 |

According to Comparative examples 113 to 116, the preparation including netarsudil dimesylate, metipranolol hydrochloride and travoprost has incompatibility under the storage condition of 5° C., and netarsudil dimesylate is precipitated.

### Comparative Examples 117 to 120: A Preparation Including Netarsudil Dimesylate, Metipranolol Hydrochloride and Tafluprost

TABLE 459
Preparation of a local ophthalmic medicine composition solution
for reducing an intra-ocular pressure as follows:

| | | Comparative | | Comparative | | Comparative | | Comparative | |
|---|---|---|---|---|---|---|---|---|---|
| No. | Component | example 117 | | example 118 | | example 119 | | example 120 | |
| 1. | Netarsudil dimesylate | 14.2 | mg | 14.2 | mg | 28.5 | mg | 28.5 | mg |
| 2. | Metipranolol hydrochloride | 111.8 | mg | 335.4 | mg | 111.8 | mg | 335.4 | mg |
| 3. | Tafluprost | 1.5 | mg | 1.5 | mg | 1.5 | mg | 1.5 | mg |
| 4. | Boric acid | 50 | mg | 50 | mg | 50 | mg | 50 | mg |
| 5. | Mannitol | 4500 | mg | 4500 | mg | 4500 | mg | 4500 | mg |
| 6. | Benzalkonium chloride | 20 | mg | 20 | mg | 20 | mg | 20 | mg |
| 7. | Sodium hydroxide | q.s. | | q.s. | | q.s. | | q.s. | |
| 8. | Water for injection added | 100 | mL | 100 | mL | 100 | mL | 100 | mL |

by

A preparation process may refer to the preparation process in Embodiments 83 to 87.

TABLE 460
Contents of various main medicine components in Comparative
examples 117 to 120 after being stored at 5° C. for 7 days

| | | Comparative | Comparative | Comparative | Comparative |
|---|---|---|---|---|---|
| | | example 117 | example 118 | example 119 | example 120 |
| No. | Component | Content % | | | |
| 1. | Netarsudil dimesylate | <30 | <30 | <30 | <30 |
| 2. | Metipranolol hydrochloride | 100.3 | 100.3 | 99.7 | 100.2 |
| 3. | Tafluprost | 99.1 | 99.4 | 99.1 | 99.3 |

According to Comparative examples 117 to 120, the preparation including netarsudil dimesylate, metipranolol hydrochloride and tafluprost has incompatibility under the storage condition of 5° C., and netarsudil dimesylate is precipitated.

1. An ophthalmic dual compound medicine composition, comprising:
(1) a β-adrenergic receptor blocker, selected from (S)-1-(tert-butylamino)-3-[(4-morpholin-1,2,5-thiadiazol-3-yl)oxy]-2-propanol (timolol) or pharmaceutically acceptable salts thereof, 5-[3-[(1,1-dimethylethyl)amino]-2-hydroxypropoxy]-3,4-dihydro-2(1H)-quinolone (carteolol) or pharmaceutically acceptable salts thereof, (RS)-1-[4-[2-(cyclopropylmethoxy)ethyl]phenoxy]3-[(1-methyl ethyl)-ammonia]-2-propanol (betaxolol) or pharmaceutically acceptable salts thereof, or 4-[2-hydroxy-3-[(1-methyl ethyl)amino]propoxy]-2,3,6-trimethyl-phenol1-acetate (metipranolol) or pharmaceutically acceptable salts thereof;
(2) (S)-2,4-dimethylbenzoic acid4-(3-amino-1-(isoquinoline-6-ylamino)-1-oxoprop-2-yl)benzyl ester (netarsudil) or pharmaceutically acceptable salts thereof;
(3) a preservative, selected from benzalkonium chloride, thimerosal, chlorbutanol, methyl p-hydroxybenzoate, propyl p-hydroxybenzoate, phenethyl alcohol, edetate disodium, boric acid, sorbic acid or any combination thereof;
(4) a buffering agent, selected from boric acid or salts thereof, sodium dihydrogen phosphate dihydrate, sodium dihydrogen phosphate monohydrate, sodium phosphate anhydrate, citric acid or salts thereof, gluconic acid or salts thereof, acetic acid or salts thereof, phosphoric acid or salts thereof, various amino acids such as glutamic acid and s-aminocaproic acid, a tris(hydroxymethyl) aminomethane buffer, or any combination thereof;
(5) a tonicity agent, selected from glycerol, sorbitol, mannitol, propylene glycol, erythritol, arabitol, xylitol, ribitol, galactitol, polyethylene glycol, lactitol and other sugar alcohol, sodium chloride, potassium chloride and calcium chloride, or any combination thereof;
(6) a pH regulator, selected from sodium hydroxide, potassium hydroxide, sodium carbonate, sodium hydrogen carbonate, hydrochloric acid, citric acid or salts thereof, phosphoric acid or salts thereof, acetic acid or salts thereof, and tartaric acid and/or hydrochloric acid or salts thereof; and
(7) water for injection.

2. An ophthalmic triple compound medicine composition, comprising:
(1) a β-adrenergic receptor blocker, selected from (S)-1-(tert-butylamino)-3-[(4-morpholin-1,2,5-thiadiazol-3-yl)oxy]-2-propanol (timolol) or pharmaceutically acceptable salts thereof, 5-[3-[(1,1-dimethylethyl)amino]-2-hydroxypropoxy]-3,4-dihydro-2(1H)-quinolone (carteolol) or pharmaceutically acceptable salts thereof, (RS)-1-[4-[2-(cyclopropylmethoxy)ethyl]phenoxy]3-[(1-methyl ethyl)-ammonia]-2-propanol (betaxolol) or pharmaceutically acceptable salts thereof, or 4-[2-hydroxy-3-[(1-methyl ethyl)amino]propoxy]-2,3,6-trimethyl-phenol1-acetate (metipranolol) or pharmaceutically acceptable salts thereof;
(2) (S)-2,4-dimethylbenzoic acid4-(3-amino-1-(isoquinoline-6-ylamino)-1-oxoprop-2-yl)benzyl ester (netarsudil) or pharmaceutically acceptable salts thereof;
(3) a prostaglandin analog, selected from latanoprost, bimatoprost, travoprost, tafluprost, AR-102, cloprostenol isopropyl ester, 13,14-dihydrocloprostenol isopropyl ester, latanoprostene bunod, unoprostone, PGF1α isopropyl ester, PGF2α isopropyl ester, PGF3α isopropyl ester or fluprostenol isopropyl ester;
(4) a preservative, selected from benzalkonium chloride, thimerosal, chlorbutanol, methyl p-hydroxybenzoate, propyl p-hydroxybenzoate, phenethyl alcohol, edetate disodium, boric acid, sorbic acid or any combination thereof;
(5) a buffering agent, selected from boric acid or salts thereof, sodium dihydrogen phosphate dihydrate, sodium dihydrogen phosphate monohydrate, sodium phosphate anhydrate, citric acid or salts thereof, gluconic acid or salts thereof, acetic acid or salts thereof, phosphoric acid or salts thereof, various amino acids such as glutamic acid and s-aminocaproic acid, tris(hydroxymethyl) aminomethane buffer, or any combination thereof;
(6) a tonicity agent, selected from glycerol, sorbitol, mannitol, propylene glycol, erythritol, arabitol, xylitol, ribitol, galactitol, polyethylene glycol, lactitol and other sugar alcohol, sodium chloride, potassium chloride and calcium chloride, or any combination thereof;
(7) a pH regulator, selected from sodium hydroxide, potassium hydroxide, sodium carbonate, sodium hydrogen carbonate, sodium hydrogen carbonate, hydrochloric acid, citric acid or salts thereof, phosphoric acid or salts thereof, acetic acid or salts thereof, and tartaric acid and/or hydrochloric acid or salts thereof; and
(8) water for injection.

3. The compound medicine composition according to claim 1, wherein the timolol is in a form of free alkali or any pharmaceutically acceptable salt (except maleate) thereof in a case that the netarsudil is dimesylate, preferably, timolol mesylate, timolol sulfate, timolol hydrobromide, timolol phosphate, timolol nitrate, timolol citrate, timolol tartrate, timolol salicylate, timolol malate, timolol lactate, timolol phenylacetate, timolol succinate, timolol hydriodate, timolol formate, timolol acetate, timolol benzoate, timolol esilate, timolol oxalate or timolol propionate; the carteolol is in a form of free alkali or any pharmaceutically acceptable salt (except hydrochloride) thereof, preferably, carteolol mesylate, carteolol hydrobromide, carteolol sulfate, carteolol esilate, carteolol nitrate, carteolol citrate, carteolol tartrate, carteolol salicylate, carteolol malate, carteolol lactate, carteolol phenylacetate, carteolol succinate, carteolol hydriodate, carteolol formate, carteolol acetate, carteolol benzoate, carteolol esilate, carteolol oxalate or carteolol propionate; the betaxolol is in a form of free alkali or any pharmaceutically acceptable salt (except hydrochloride) thereof, preferably, betaxolol mesylate, betaxolol hydrobromide, betaxolol sulfate, betaxolol esilate, betaxolol nitrate, betaxolol citrate, betaxolol tartrate, betaxolol salicylate, betaxolol malate, betaxolol lactate, betaxolol phenylacetate, betaxolol succinate, betaxolol hydriodate, betaxolol formate, betaxolol acetate, betaxolol benzoate, betaxolol esilate, betaxolol oxalate or betaxolol propionate; and the metipranolol is in a form of free alkali or any pharmaceutically acceptable salt (except hydrochloride) thereof, preferably, metipranolol mesylate, metipranolol hydrobromide, metipranolol sulfate, metipranolol esilate, metipranolol nitrate, metipranolol citrate, metipranolol tartrate, metipranolol salicylate, metipranolol malate, metipranolol lactate, metipranolol phenylacetate, metipranolol succinate, metipranolol hydriodate, metipranolol formate, metipranolol acetate, metipranolol benzoate, metipranolol esilate, metipranolol oxalate or metipranolol propionate.

4. The compound medicine composition according to claim 1, wherein the netarsudil is in a form of free alkali or any pharmaceutically acceptable salt (except mesylate) thereof in a case that the β-adrenergic receptor blocker is timolol maleate, or carteolol hydrochloride, or betaxolol hydrochloride, or metipranolol hydrochloride, preferably, netarsudil maleate, netarsudil sulfate, netarsudil dihydrobromide, netarsudil dihydrochloride, netarsudil diformate, netarsudil dinitrate, netarsudil diacetate, netarsudil dibenzoate, netarsudil diphenylacetate, netarsudil succinate, netarsudil oxalate, netarsudil dihydriodate, or netarsudil dipropionate.

5. The compound medicine composition according to claim 1, comprising:
(1) 0.02% w/v to 4.0% w/v β-adrenergic receptor blocker, preferably, timolol free alkali or salts thereof, carteolol free alkali or salts thereof, betaxolol free alkali or salts thereof, or metipranolol free alkali or salts thereof;
(2) 0.005% w/v to 0.1% w/v netarsudil free alkali or salts thereof;
(3) 0.01% w/v to 10.0% ow/v tonicity agent;
(4) 0.01% w/v to 1.0% w/v buffering agent; and
(5) 0.001% w/v to 0.02% w/v preservative;
(6) wherein pH is in a range from 4.5 to 5.4; and
(7) an osmotic pressure is in a range from 280 mOsmol/kg to 320 mOsmol/kg.

6. The compound medicine composition according to claim 2, comprising:
(1) 0.02% w/v to 4.0% w/v β-adrenergic receptor blocker, preferably, timolol free alkali or salts thereof, carteolol free alkali or salts thereof, betaxolol free alkali or salts thereof, or metipranolol free alkali or salts thereof;
(2) 0.005% w/v to 0.1% w/v netarsudil free alkali or salts thereof;
(3) 0.0005% w/v to 0.05% w/v prostaglandin analog;
(4) 0.01% w/v to 10.0% w/v tonicity agent;
(5) 0.01% w/v to 1.0% ow/v buffering agent;
(6) 0.001% w/v to 0.02% w/v preservative;
(7) wherein pH is in a range from 4.5 to 5.4; and
(8) an osmotic pressure is in a range from 280 mOsmol/kg to 320 mOsmol/kg.

7. The compound medicine composition according to claim 1, wherein the medicine composition does not comprise precipitate after being stored at 5° C. for 24 months, and a content of each main medicine component has no significant change compared with zero day; the medicine composition does not comprise precipitate after being stored at 25° C. for 6 weeks, and a content of each main medicine component has no significant change compared with zero day; and the medicine composition does not comprise precipitate after being stored at 40° C. for 14 days, and a content of each main medicine component has no significant change compared with zero day.

8. Use of the compound medicine composition according to claim 1 in preparation of medicine for preventing or treating an eye disease.

9. The use according to claim 8, wherein the eye disease is glaucoma or symptoms related thereto.

10. Use of the compound medicine composition according to claim 1 in preparation of medicine for reducing an intra-ocular pressure.
